SEQUENCE LISTING (75 sequences)

SEQ ID NO: 1 - protein, 394 aa, synthetic construct
MKNCVIVSAVRTAIGSFNGSLASTSAIDLGATVIKAAIERAKIDSQHVDEVIMGNVLQAGLGQNPARQALLKSGLAETVCGFTVNKVCGSGLKSVALAAQAIQAGQAQSIVAGGMENMSLAPYLLDAKARSGYRLGDGQVYDVILRDGLMCATHGYHMGITAENVAKEYGITREMQDELALHSQRKAAAAIESGAFTAEIVPVNVVTRKKTFVFSQDEFPKANSTAEALGALRPAFDKAGTVTAGNASGINDGAAALVIMEESAALAAGLTPLARIKSYASGGVPPALMGMGPVPATQKALQLAGLQLADIDLIEANEAFAAQFLAVGKNLGFDSEKVNVNGGAIALGHPIGASGARILVTLLHAMQARDKTLGLATLCIGGGQGIAMVIERLN

SEQ ID NO: 2 - protein, 383 aa, synthetic construct
MTIGIDKISFFVPPYYIDMTALAEARNVDPGKFHIGIGQDQMAVNPISQDIVTFAANAAEAILTKEDKEAIDMVIVGTESSIDESKAAAVVLHRLMGIQPFARSFEIKEACYGATAGLQLAKNHVALHPDKKVLVVAADIAKYGLNSGGEPTQGAGAVAMLVASEPRILALKEDNVMLTQDIYDFWRPTGHPYPMVDGPLSNETYIQSFAQVWDEHKKRTGLDFADYDALAFHIPYTKMGKKALLAKISDQTEAEQERILARYEESIIYSRRVGNLYTGSLYLGLISLLENATTLTAGNQIGLFSYGSGAVAEFFTGELVAGYQNHLQKETHLALLDNRTELSIAEYEAMFAETLDTDIDQTLEDELKYSISAINNTVRSYRN

SEQ ID NO: 3 - protein, 803 aa, synthetic construct
MKTVVIIDALRTPIGKYKGSLSQVSAVDLGTHVTTQLLKRHSTISEEIDQVIFGNVLQAGNGQNPARQIAINSGLSHEIPAMTVNEVCGSGMKAVILAKQLIQLGEAEVLIAGGIENMSQAPKLQRFNYETESYDAPFSSMMYDGLTDAFSGQAMGLTAENVAEKYHVTREEQDQFSVHSQLKAAQAQAEGIFADEIAPLEVSGTLVEKDEGIRPNSSVEKLGTLKTVFKEDGTVTAGNASTINDGASALIIASQEYAEAHGLPYLAIIRDSVEVGIDPAYMGISPIKAIQKLLARNQLTTEEIDLYEINEAFAATSIVVQRELALPEEKVNIYGGGISLGHAIGATGARLLTSLSYQLNQKEKKYGVASLCIGGGLGLAMLLERPQQKKNSRFYQMSPEERLASLLNEGQISADTKKEFENTALSSQIANHMIENQISETEVPMGVGLHLTVDETDYLVPMATEEPSVIAALSNGAKIAQGFKTVNQQRLMRGQIVFYDVADAESLIDELQVRETEIFQQAELSYPSIVKRGGGLRDLQYRAFDESFVSVDFLVDVKDAMGANIVNAMLEGVAELFREWFAEQKILFSILSNYATESVVTMKTAIPVSRLSKGSNGREIAEKIVLASRYASLDPYRAVTHNKGIMNGIEAVVLATGNDTRAVSASCHAFAVKEGRYQGLTSWTLDGEQLIGEISVPLALATVGGATKVLPKSQAAADLLAVTDAKELSRVVAAVGLAQNLAALRALVSEGIQKGHMALQARSLAMTVGATGKEVEAVAQQLKRQKTMNQDRALAILNDLRKQ

SEQ ID NO: 4 - protein, 443 aa, synthetic construct
MSLPFLTSAPGKVIIFGEHSAVYNKPAVAASVSALRTYLLISESSAPDTIELDFPDISFNHKWSINDFNAITEDQVNSQKLAKAQQATDGLSQELVSLLDPLLAQLSESFHYHAAFCFLYMFVCLCPHAKNIKFSLKSTLPIGAGLGSSASISVSLALAMAYLGGLIGSNDLEKLSENDKHIVNQWAFIGEKCIHGTPSGIDNAVATYGNALLFEKDSHNGTINTNNFKFLDDFPAIPMILTYTRIPRSTKDLVARVRVLVTEKFPEVMKPILDAMGECALQGLEIMTKLSKCKGTDDEAVETNNELYEQLLELIRINHGLLVSIGVSHPGLELIKNLSDDLRIGSTKLTGAGGGGCSLTLLRRDITQEQIDSFKKKLQDDFSYETFETDLGGTGCCLLSAKNLNKDLKIKSLVFQLFENKTTTKQQIDDLLLPGNTNLPWTS

SEQ ID NO: 5 - protein, 451 aa, synthetic construct
MSELRAFSAPGKALLAGGYLVLDTKYEAFVVGLSARMHAVAHPYGSLQGSDKFEVRVKSKQFKDGEWLYHISPKSGFIPVSIGGSKNPFIEKVIANVFSYFKPNMDDYCNRNLFVIDIFSDDAYHSQEDSVTEHRGNRRLSFHSHRIEEVPKTGLGSSAGLVTVLTTALASFFVSDLENNVDKYREVIHNLAQVAHCQAQGKIGSGFDVAAAAYGSIRYRRFPPALISNLPDIGSATYGSKLAHLVDEEDWNITIKSNHLPSGLTLWMGDIKNGSETVKLVQKVKNWYDSHMPESLKIYTELDHANSRFMDGLSKLDRLHETHDDYSDQIFESLERNDCTCQKYPEITEVRDAVATIRRSFRKITKESGADIEPPVQTSLLDDCQTLKGVLTCLIPGAGGYDAIAVITKQDVDLRAQTANDKRFSKVQWLDVTQADWGVRKEKDPETYLDK

SEQ ID NO: 6 - protein, 396 aa, synthetic construct
MTVYTASVTAPVNIATLKYWGKRDTKLNLPTNSSISVTLSQDDLRTLTSAATAPEFERDTLWLNGEPHSIDNERTQNCLRDLRQLRKEMESKDASLPTLSQWKLHIVSENNFPTAAGLASSAAGFAALVSAIAKLYQLPQSTSEISRIARKGSGSACRSLFGGYVAWEMGKAEDGHDSMAVQIADSSDWPQMKACVLVVSDIKKDVSSTQGMQLTVATSELFKERIEHVVPKRFEVMRKAIVEKDFATFAKETMMDSNSFHATCLDSFPPIFYMNDTSKRIISWCHTINQFYGETIVAYTFDAGPNAVLYYLAENESKLFAFIYKLFGSVPGWDKKFTTEQLEAFNHQFESSNFTARELDLELQKDVARVILTQVGSGPQETNESLIDAKTGLPKE

SEQ ID NO: 7 - protein, 182 aa, synthetic construct
MQTEHVILLNAQGVPTGTLEKYAAHTADTRLHLAFSSWLFNAKGQLLVTRRALSKKAWPGVWTNSVCGHPQLGESNEDAVIRRCRYELGVEITPPESIYPDFRYRATDPSGIVENEVCPVFAARTTSALQINDDEVMDYQWCDLADVLHGIDATPWAFSPWMVMQATNREARKRLSAFTQLK

SEQ ID NO: 8 - protein, 297 aa, synthetic construct
MEFDFNKYMDSKAMTVNEALNKAIPLRYPQKIYESMRYSLLAGGKRVRPVLCIAACELVGGTEELAIPTACAIEMIHTMSLMHDDLPCIDNDDLRRGKPTNHKIFGEDTAVTAGNALHSYAFEHIAVSTSKTVGADRILRMVSELGRATGSEGVMGGQMVDIASEGDPSIDLQTLEWIHIHKTAMLLECSVVCGAIIGGASEIVIERARRYARCVGLLFQVVDDILDVTKSSDELGKTAGKDLISDKATYPKLMGLEKAKEFSDELLNRAKGELSCFDPVKAAPLLGLADYVAFRQN

SEQ ID NO: 9 - protein, 329 aa, synthetic construct
MQEFEFAVPAPSRVSPDLARARARHLDWVHAMDLVRGEEARRRYEFSCVADIGAYGYPHATGADLDLCVDVLGWTFLFDDQFDAGDGRERDALAVCAELTDLLWKGTAATAASPPIVVAFSDCWERMRAGMSDAWRRRTVHEWVDYLAGWPTKLADRAHGAVLDPAAHLRARHRTICCRPLFALAERVGGYEVPRRAWHSSRLDGMRFTTSDAVIGMNELHSFEKDRAQGHANLVLSLVHHGGLTGPEAVTRVCDLVQGSIESFLRLRSGLPELGRALGVEGAVLDRYADALSAFCRGYHDWGRGASRYTTRDHPGDLGLENLVARSSG

SEQ ID NO: 10 - protein, 330 aa, synthetic construct
MPAGHEEFDIPFPSRVNPFHARAEDRHVAWMRAMGLITGDAAEATYRRWSPAKVGARWFYLAQGEDLDLGCDIFGWFFAYDDHFDGPTGTDPRQTAAFVNRTVAMLDPRADPTGEHPLNIAFHDLWQRESAPMSPLWQRRAVDHWTQYLTAHITEATNRTRHTSPTIADYLELRHRTGFMPPLLDLIERVWRAEIPAPVYTTPEVQTLLHTTNQNINIVNDVLSLEKEEAHGDPHNLVLVIQHERQSTRQQALATARRMIDEWTDTFIRTEPRLPALCGRLGIPLADRTSLYTAVEGMRAAIRGNYDWCAETNRYAVHRPTGTGRATTPW

SEQ ID NO: 11 - protein, 628 aa, synthetic construct
MALVSAVPLNSKLCLRRTLFGFSHELKAIHSTVPNLGMCRGGKSIAPSMSMSSTTSVSNEDGVPRRIAGHHSNLWDDDSIASLSTSYEAPSYRKRADKLIGEVKNIFDLMSVEDGVFTSPLSDLHHRLWMVDSVERLGIDRHFKDEINSALDHVYSYWTEKGIGRGRESGVTDLNSTALGLRTLRLHGYTVSSHVLDHFKNEKGQFTCSAIQTEGEIRDVLNLFRASLIAFPGEKIMEAAEIFSTMYLKDALQKIPPSGLSQEIEYLLEFGWHTNLPRMETRMYIDVFGEDTTFETPYLIREKLLELAKLEFNIFHSLVKRELQSLSRWWKDYGFPEITFSRHRHVEYYTLAACIANDPKHSAFRLGFGKISHMITILDDIYDTFGTMEELKLLTAAFKRWDPSSIECLPDYMKGVYMAVYDNINEMAREAQKIQGWDTVSYARKSWEAFIGAYIQEAKWISSGYLPTFDEYLENGKVSFGSRITTLEPMLTLGFPLPPRILQEIDFPSKFNDLICAILRLKGDTQCYKADRARGEEASAVSCYMKDHPGITEEDAVNQVNAMVDNLTKELNWELLRPDSGVPISYKKVAFDICRVFHYGYKYRDGFSVASIEIKNLVTRTVVETVPL

SEQ ID NO: 12 - protein, 628 aa, synthetic construct
MALVSTAPLASKSCLHKSLISSTHELKALSRTIPALGMSRRGKSITPSISMSSTTVVTDDGVRRRMGDFHSNLWDDDVIQSLPTAYEEKSYLERAEKLIGEVKNMFNSMSLEDGELMSPLNDLIQRLWIVDSLERLGIHRHFKDEIKSALDYVYSYWGENGIGCGRESVVTDLNSTALGLRTLRLHGYPVSSDVFKAFKGQNGQFSCSENIQTDEEIRGVLNLFRASLIAFPGEKIMDEAEIFSTKYLKEALQKIPVSSLSREIGDVLEYGWHTYLPRLEARNYIQVFGQDTENTKSYVKSKKLLELAKLEFNIFQSLQKRELESLVRWWKESGFPEMTFCRHRHVEYYTLASCIAFEPQHSGFRLGFAKTCHLITVLDDMYDTFGTVDELELFTATMKRWDPSSIDCLPEYMKGVYIAVYDTVNEMAREAEEAQGRDTLTYAREAWEAYIDSYMQEARWIATGYLPSFDEYYENGKVSCGHRISALQPILTMDIPFPDHILKEVDFPSKLNDLACAILRLRGDTRCYKADRARGEEASSISCYMKDNPGVSEEDALDHINAMISDVIKGLNWELLKPDINVPISAKKHAFDIARAFHYGYKYRDGYSVANVETKSLVTRTLLESVPL

SEQ ID NO: 13 - protein, 542 aa, synthetic construct
MRRSGNYQAPVWNNDFIQSFSTDKYKDEKFLKKKEELIAQVKVLLNTKMEAVKQLELIEDLRNLGLTYYFEDEFKKILTSIYNEHKGFKNEQVGDLYFTSLAFRLLRLHGFDVSEDVFNFFKNEDGSDFKASLGENTKDVLELYEASFLIRVGEVTLEQARVFSTKILEKKVEEGIKDEKLLAWIQHSLALPLHWRIQRLEARWFLDAYKARKDMNPIIYELGKIDFHIIQETQLQEVQEVSQWWTNTNLAEKLPFVRDRIVECYFWALGLFEPHEYGYQRKMAAIIITFVTIIDDVYDVYDTLDELQLFTDAIRKWDVESISTLPYYMQVCYLAVFTYASELAYDILKDQGFNSISYLQRSWLSLVEGFFQEAKWYYAGYTPTLAEYLENAKVSISSPTIISQVYFTLPNSTERTVVENVFGYHNILYLSGMILRLADDLGTTQFELKRGDVQKAIQCYMNDNNATEEEGTEHVKYLLREAWQEMNSAMADPDCPLSEDLVFAAANLGRTSQFIYLDGDGHGVQHSEIHNQMGGLIFEPYV

SEQ ID NO: 14 - protein, 567 aa, synthetic construct
MSCARITVTLPYRSAKTSIQRGITHYPALIRPRFSACTPLASAMPLSSTPLINGDNSQRKNTRQHMEESSSKRREYLLEETTRKLQRNDTESVEKLKLIDNIQQLGIGYYFEDAINAVLRSPFSTGEEDLFTAALRFRLLRHNGIEISPEIFLKFKDERGKFDESDTLGLLSLYEASNLGVAGEEILEEAMEFAEARLRRSLSEPAAPLHGEVAQALDVPRHLRMARLEARRFIEQYGKQSDHDGDLLELAILDYNQVQAQHQSELTEIIRWWKELGLVDKLSFGRDRPLECFLWTVGLLPEPKYSSVRIELAKAISILLVIDDIFDTYGEMDDLILFTDAIRRWDLEAMEGLPEYMKICYMALYNTTNEVCYKVLRDTGRIVLLNLKSTWIDMIEGFMEEAKWFNGGSAPKLEEYIENGVSTAGAYMAFAHIFFLIGEGVTHQNSQLFTQKPYPKVFSAAGRILRLWDDLGTAKEEQERGDLASCVQLFMKEKSLTEEEARSRILEEIKGLWRDLNGELVYNKNLPLSIIKVALNMARASQVVYKHDQDTYFSSVDNYVDALFFTQ

SEQ ID NO: 15 - protein, 606 aa, synthetic construct
MSSCINPSTLVTSVNAFKCLPLATNKAAIRIMAKYKPVQCLISAKYDNLTVDRRSANYQPSIWDHDFLQSLNSNYTDEAYKRRAEELRGKVKIAIKDVIEPLDQLELIDNLQRLGLAHRFETEIRNILNNIYNNNKDYNWRKENLYATSLEFRLLRQHGYPVSQEVFNGFKDDQGGFICDDFKGILSLHEASYYSLEGESIMEEAWQFTSKHLKEVMISKNMEEDVFVAEQAKRALELPLHWKVPMLEARWFIHIYERREDKNHLLLELAKMEFNTLQAIYQEELKEISGWWKDTGLGEKLSFARNRLVASFLWSMGIAFEPQFAYCRRVLTISIALITVIDDIYDVYGTLDELEIFTDAVERWDINYALKHLPGYMKMCFLALYNFVNEFAYYVLKQQDFDLLLSIKNAWLGLIQAYLVEAKWYHSKYTPKLEEYLENGLVSITGPLIITISYLSGTNPIIKKELEFLESNPDIVHWSSKIFRLQDDLGTSSDEIQRGDVPKSIQCYMHETGASEEVARQHIKDMMRQMWKKVNAYTADKDSPLTGTTTEFLLNLVRMSHFMYLHGDGHGVQNQETIDVGFTLLFQPIPLEDKHMAFTASPGTKG

SEQ ID NO: 16 - protein, 599 aa, synthetic construct
MALKVLSVATQMAIPSNLTTCLQPSHFKSSPKLLSSTNSSSRSRLRVYCSSSQLTTERRSGNYNPSRWDVNFIQSLLSDYKEDKHVIRASELVTLVKMELEKETDQIRQLELIDDLQRMGLSDHFQNEFKEILSSIYLDHHYYKNPFPKEERDLYSTSLAFRLLREHGFQVAQEVFDSFKNEEGEFKESLSDDTRGLLQLYEASFLLTEGETTLESAREFATKFLEEKVNEGGVDGDLLTRIAYSLDIPLHWRIKRPNAPVWIEWYRKRPDMNPVVLELAILDLNIVQAQFQEELKESFRWWRNTGFVEKLPFARDRLVECYFWNTGIIEPRQHASARIMMGKVNALITVIDDIYDVYGTLEELEQFTDLIRRWDINSIDQLPDYMQLCFLALNNFVDDTSYDVMKEKGVNVIPYLRQSWVDLADKYMVEARWFYGGHKPSLEEYLENSWQSISGPCMLTHIFFRVTDSFTKETVDSLYKYHDLVRWSSFVLRLADDLGTSVEEVSRGDVPKSLQCYMSDYNASEAEARKHVKWLIAEVWKKMNAERVSKDSPFGKDFIGCAVDLGRMAQLMYHNGDGHGTQHPIIHQQMTRTLFEPFA

SEQ ID NO: 17 - protein, 629 aa, synthetic construct
MAVHQFSPSIVSSLPTISTYNNNHFCRFFTPKTSISPISKTKSKSSTCYPIQCTVVNNSSPSSTIVRRSANYEPPIWSFDYIQSLSTQYKGESYTRQLNKLKKEVKRMLLRMEINSLALLELIDTLQRLGISYHFKNEINTILKKKYNDNYINNNIITNPNYNNLYATALEFRLLRQHGYTVPQEIFNAFKDKRGKFKTSLSDDIMGVLCLYEASFYAMKHENILEEARIFSTKCLKKYMEKIENEEEKKILLLNDNNINSNLLLINHAFELPLHWRITRSEARWFIDEIYEKKQDMNSTLFEFAKLDFNIVQSTHQEDLQHLSRWWRDCKLGGKLNFARDRLMEAFLWDVGLKFEGEFSYFRRTNARLFVLITIIDDIYDVYGTLEELELFTSAVERWDVKLINELPDYMKMPFFVLHNTINEMGFDVLVEQNFVNIEYLKKSWVDLCKCYLQEAKWYYSGYQPTLEEYTELGWLSIGASVILMHAYFCFTNPITKQDLKSLQLQHHYPNIIKQACLITRLADDLGTSSDELNRGDVPKSIQCYMYDNNATEDEAREHIKFLISETWKDMNKKDEDESCLSENFVEVCKNMARTALFIYENGDGHGSQNSLSKERISTLIITPINIPK

SEQ ID NO: 18 - protein, 541 aa, synthetic construct
MRRSANYQPSLWQHEYLLSLGNTYVKEDNVERVTLLKQEVSKMLNETEGLLEQLELIDTLQRLGVSYHFEQEIKKTLTNVHVKNVRAHKNRIDRNRWGDLYATALEFRLLRQHGFSIAQDVFDGNIGVDLDDKDIKGILSLYEASYLSTRIDTKLKESIYYTTKRLRKFVEVNKNETKSYTLRRMVIHALEMPYHRRVGRLEARWYIEVYGERHDMNPILLELAKLDFNFVQAIHQDELKSLSSWWSKTGLTKHLDFVRDRITEGYFSSVGVMYEPEFAYHRQMLTKVFMLITTIDDIYDIYGTLEELQLFTTIVEKWDVNRLEELPNYMKLCFLCLVNEINQIGYFVLRDKGFNVIPYLKESWADMCTTFLKEAKWYKSGYKPNFEEYMQNGWISSSVPTILLHLFCLLSDQTLDILGSYNHSVVRSSATILRLANDLATSSEELARGDTMKSVQCHMHETGASEAESRAYIQGIIGVAWDDLNMEKKSCRLHQGFLEAAANLGRVAQCVYQYGDGHGCPDKAKTVNHVRSLLVHPLPLN

SEQ ID NO: 19 - protein, 581 aa, synthetic construct
MPLNSLHNLERKPSKAWSTSCTAPAARLQASFSLQQEEPRQIRRSGDYQPSLWDFNYIQSLNTPYKEQRYVNRQAELIMQVRMLLKVKMEAIQQLELIDDLQYLGLSYFFPDEIKQILSSIHNEHRYFHNNDLYLTALGFRILRQHGFNVSEDVFDCFKTEKCSDFNANLAQDTKGMLQLYEASFLLREGEDTLELARRFSTRSLREKLDEDGDEIDEDLSSWIRHSLDLPLHWRIQGLEARWFLDAYARRPDMNPLIFKLAKLNFNIVQATYQEELKDVSRWWNSSCLAEKLPFVRDRIVECFFWAIGAFEPHQYSYQRKMAAIIITFVTIIDDVYDVYGTLEELELFTDMIRRWDNISISQLPYYMQVCYLALYNFVSERAYDILKDQHFNSIPYLQRSWVSLVEGYLKEAYWYYNGYKPSLEEYLNNAKISISAPTIISQLYFTLANSTDETVIESLYEYHNILYLSGTILRLADDLGTSQHELERGDVPKAIQCYMKDTNASEREAVEHVKFLIRETWKEMNTVTTASDCPFTDDLVAVATNLARAAQFIYLDGDGHGVQHSEIHQQMGGLLFQPYV

SEQ ID NO: 20 - protein, 569 aa, synthetic construct
MSNFLVSTCSSPLALDENNSTKQDHVIRNTVTFHSSIWGDQFLTYDEKDDLVAEKQLAEELIEETRKELIITTSSHEPIQHMKLIQLIDAVQRLGVAYHFEKEIEDALQHVYRTYGHQGIHNNNDLQSISLWFRILRQQGFNVSSEIFKNHMDEKGNLFSNDVQSMLALYEASYMRVEGEKVLDDALEFTKTHLAIIAKDPSCDSSLRTQIQDALRQPLRKRLPRLEAVRYIPIYQQQSSHNQILLKLAKLDFNMLQTMHKKELSEICKWWKDLDMQNKLPFVRDRLIEGYFWILGIYFEPHHSRSRMFLIKSCMWLVVIDDTFDNYGTYEELEIFTEAVERWSINCLDMLPEYMKLIYKELVIVHQEMEETLEKEGKAYHIHHVKELAKECTRSLLVEAKWLKEGYMPTLDEYISNSLITCAYAVMIARSYVGGDDKLVNEDSFKWVATHPPLVKASCLILRLMDDIATHKEEQERGHVASSIECYIKETGATEEEAREHFSKQVEDAWKVVNRESLRPTAVAFPLVMPAINLARMCDALYKGNHDGYNHAGKEVIQYIKSLLVHPLI

SEQ ID NO: 21 - protein, 299 aa, synthetic construct
MDFPQQLEACVKQANQALSRFIAPLPFQNTPVVETMQYGALLGGKRLRPFLVYATGHMFGVSTNTLDAPAAAVECIHAYSLIHDDLPAMDDDDLRRGLPTCHVKFGEANAILAGDALQTLAFSILSDADMPEVSDRDRISMISELASASGIAGMCGGQALDLDAEGKHVPLDALERIHRHKTGALIRAAVRLGALSAGDKGRRALPVLDKYAESIGLAFQVQDDILDVVGDTATLGKRQGADQQLGKSTYPALLGLEQARKKARDLIDDARQSLKQLAEQSLDTSALEALADYIIQRNK

SEQ ID NO: 22 - protein, 254 aa, synthetic construct
MRSIARRTAVGAALLLVMPVAVWISGWRWQPGEQSWLLKAAFWVTETVTQPWGVITHLILFGWFLWCLRFRIKAAFVLFAILAAAILVGQGVKSWIKDKVQEPRPFVIWLEKTHHIPVDEFYTLKRAERGNLVKEQLAEEKNIPQYLRSHWQKETGFAFPSGHTMFAASWALLAVGLLWPRRRTLTIAILLVWATGVMGSRLLLGMHWPRDLVVATLISWALVAVATWLAQRICGPLTPPAEENREIAQREQES

SEQ ID NO: 23 - protein, 580 aa, synthetic construct
MAQISIGAPLSAEVNGACINTHHHGNLWDDYFIQSLKSPYEAPECHERCEKMIEEVKHLLLSEMRDGNDDLIKRLQMVDIFECLGIDRHFHHEIQAALDYVYRYWNELEGIGVGTRDSLTKDLYATGLGFRALRLHRYNVSSAVLENFKNENGLFFHSSAVQEEEVRCMLTLLRASEISFPGEKVMDEAKAFATEYLNQLLTRVDITEVGENLLREVRYALDFPWYCSVPRWEARSFIEIFGQNNSWLKSTMNKKVLELAKLDFNILQSAHQRELQLLSRWWSQSDIEKQNFYRKRHVEFYFWMVIGTFEPEFSSSRIAFAKIATLMTILDDLYDTHGTLEQLKIFTEAVKRWDLSLQDRLPDYIKITLEFFFNTSNELNAEVAKMQERDMSAYIRKAGWERYIEGYMQESEWMAARHVPTFDDYMKNGKRSSGMCILNLYSLLLMGQLVPDNILEQIHLPSKIHELVELTARLVDDSKDFQAKKDGGEFASGTECYLKEKPECTEEDAMNHLIGLLNLTAMELNWEFVKHDGVALCLKKFVFEVARGLRFIYKYRDGFDYSNEEMKSQITKILIDQVPI

SEQ ID NO: 24 - protein, 589 aa, synthetic construct
MAEMFNGNSSNDGSSCMPVKDALRRTGNHHPNLWTDDFIQSLNSPYSDSSYHKHREILIDEIRDMFSNGEGDEFGVLENIWFVDVVQRLGIDRHFQEEIKTALDYIYKFWNHDSIFGDLNMVALGFRILRLNRYVASSDVFKKFKGEEGQFSGFESSDQDAKLEMMLNLYKASELDFPDEDILKEARAFASMYLKHVIKEYGDIQESKNPLLMEIEYTFKYPWRCRLPRLEAWNFIHIMRQQDCNISLANNLYKIPKIYMKKILELAILDFNILQSQHQHEMKLISTWWKNSSAIQLDFFRHRHIESYFWWASPLFEPEFSTCRINCTKLSTKMFLLDDIYDTYGTVEELKPFTTTLTRWDVSTVDNHPDYMKIAFNFSYEIYKEIASEAERKHGPFVYKYLQSCWKSYIEAYMQEAEWIASNHIPGFDEYLMNGVKSSGMRILMIHALILMDTPLSDEILEQLDIPSSKSQALLSLITRLVDDVKDFEDEQAHGEMASSIECYMKDNHGSTREDALNYLKIRIESCVQELNKELLEPSNMHGSFRNLYLNVGMRVIFFMLNDGDLFTHSNRKEIQDAITKFFVEPIIP

SEQ ID NO: 25 - protein, 557 aa, synthetic construct
MAAVDTNATIQEKTTAEPVRPLANFPPSVWGDRFLSFTLDNSELEGYAKAMEAPKEELRRLIVDQTMDSNKKLSLIYSVHRLGLTYLFLQEIEAQLDKIFKEFNLQNYDEVDLYTTSINFQVFRHLGYKLPCDVFNKFKDNTSGAFKEDISTDVKGMLGLYESSQLRTRGESILDEASSFTETKLKSVVNNLEGNLAQQVLQSLRRPFHQGMPMVEARLYFSNYSEECATHECLLKLAKLHFSYLELQQKEELRIVSKWWKDMRFQETTPYIRDRVPEIYLWILGLYFEPRYSLARIIATKITLFLVVLDDTYDAYATIEEIRLLTDAINRWDMSAMEQIPEYIRPFYKILLDEYAELEKQLAIEGRAKSVIASKEAFQDIARGYLEEAEWTNSGYVASFPEYMKNGLITSAYNVISKSALVGMGDIVSENALAWYESHPKTLQASELISRLQDDVMTYQFERERGQSATGVDAYIKTYGVSEKEAIDELKIMIENAWKDINEGCLKPREVSMDLLAPILNLARMIDVVYRYDDGFTFPGKTMKEYITLLFVGSVSM

SEQ ID NO: 26 - protein, 574 aa, synthetic construct
MSTLPISSVSFSSSTSPLVVDDKVSTKPDVIRHTMNFNASIWGDQFLTYDEPEDLVMKKQLVEELKEEVKKELITIKGSNEPMQHVKLIELIDAVQRLGIAYHFEEEIEEALQHIHVTYGEQWVDKENLQSISLWFRLLRQQGFNVSSGVFKDFMDEKGKFKESLCNDAQGILALYEAAFMRVEDETILDNALEFTKVHLDIIAKDPSCDSSLRTQIHQALKQPLRRRLARIEALHYMPIYQQETSHDEVLLKLAKLDFSVLQSMHKKELSHICKWWKDLDLQNKLPYVRDRVVEGYFWILSIYYEPQHARTRMFLMKTCMWLVVLDDTFDNYGTYEELEIFTQAVERWSISCLDMLPEYMKLIYQELVNLHVEMEESLEKEGKTYQIHYVKEMAKELVRNYLVEARWLKEGYMPTLEEYMSVSMVTGTYGLMIARSYVGRGDIVTEDTFKWVSSYPPIIKASCVIVRLMDDIVSHKEEQERGHVASSIECYSKESGASEEEACEYISRKVEDAWKVINRESLRPTAVPFPLLMPAINLARMCEVLYSVNDGFTHAEGDMKSYMKSFFVHPMVV

SEQ ID NO: 27 - protein, 552 aa, synthetic construct
MELYAQSVGVGAASRPLANFHQCVWGGKFIVYNPQSSQAGEREQAEELKVELKRELKEASDNYMRQLKMVDAIQRLGIDYLFVEDVDEALKNLFEMFDAFCKNNHDMHATALSFRLLRQHGYRVSCEVFEKFKDGKDGFKVPNEDGAVAVLEFFEATHLRVHGEDVLDNAFVFTRNYLESVYATLNDPTANQVHNALNEFSFRRGLPRVEARKYISIYEQYASHHKGLLRLAKLDFNLVQALHRRELSEDSRWWKTLQVPTELSFVRDRLVESYFWASGSYFEPNYSVARMILAKGLAVLSLMDDVYDAYGTFEELQVFTDAIERWDALCLDKLPEYMKIVYKALLDVFEEVDEEVIKLGAPYRVYYGKEAMKYAARAYMEEAQWREQKHKPTTKEYMKLATKTCGYITLIILSFLGVEEGIVTKEAFDWVFSRPPFVEATLIIARLINDITGCEFENKREHVRTAVECYMEEHKVGKQEVVSEFYNQMESAWKDINECLLRPAEFPIPLLNLILNSVRTLEVIYKEGDSYTHVGPAMQNIIKQLYLHPVPY

SEQ ID NO: 28 - protein, 337 aa, synthetic construct
MPQDVDFHIPLPGRQSPDHARAEAEQLAWPRSLGLIRSDAAAERHLRGGYADLASRFYPHATGADLDLGVDLMSWFFLFDDLFDGPRGENPEDTKQLTDQVAAALDGPLPDTAPPIAHGFADIWRRTCEGMTPAWCARSARHWRNYFDGYVDEAESRFWNAPCDSAAQYLAMRRHTIGVQPTVDLAERAGRFEVPHRVFDSAVMSAMLQIAVDVNLLLNDIASLEKEEARGEQNNMVMILRREHGWSKSRSVSHMQNEVRARLEQYLLLESCLPKVGEIYQLDTAEREALERYRTDAVRTVIRGSYDWHRSSGRYDAEFALAAGAQGYLEELGSSAH

SEQ ID NO: 29 - protein, 551 aa, synthetic construct
MSTQQVSSENIVRNAANFHPNIWGNHFLTCPSQTIDSWTQQHHKELKEEVRKMMVSDANKPAQRLRLIDTVQRLGVAYHFEKEIDDALEKIGHDPFDDKDDLYIVSLCFRLLRQHGIKISCDVFEKFKDDDGKFKASLMNDVQGMLSLYEAAHLAIHGEDILDEAIVFTTTHLKSTVSNSPVNSTFAEQIRHSLRVPLRKAVPRLESRYFLDIYSRDDLHDKTLLNFAKLDFNILQAMHQKEASEMTRWWRDFDFLKKLPYIRDRVVELYFWILVGVSYQPKFSTGRIFLSKIICLETLVDDTFDAYGTFDELAIFTEAVTRWDLGHRDALPEYMKFIFKTLIDVYSEAEQELAKEGRSYSIHYAIRSFQELVMKYFCEAKWLNKGYVPSLDDYKSVSLRSIGFLPIAVASFVFMGDIATKEVFEWEMNNPKIIIAAETIFRFLDDIAGHRFEQKREHSPSAIECYKNQHGVSEEEAVKALSLEVANSWKDINEELLLNPMAIPLPLLQVILDLSRSADFMYGNAQDRFTHSTMMKDQVDLVLKDPVKLDD

SEQ ID NO: 30 - protein, 596 aa, synthetic construct
MATELLCLHRPISLTHKLFRNPLPKVIQATPLTLKLRCSVSTENVSFTETETEARRSANYEPNSWDYDYLLSSDTDESIEVYKDKAKKLEAEVRREINNEKAEFLTLLELIDNVQRLGLGYRFESDIRGALDRFVSSGGFDAVTKTSLHGTALSFRLLRQHGFEVSQEAFSGFKDQNGNFLENLKEDIKAILSLYEASFLALEGENILDEAKVFAISHLKELSEEKIGKELAEQVNHALELPLHRRTQRLEAVWSIEAYRKKEDANQVLLELAILDYNMIQSVYQRDLRETSRWWRRVGLATKLHFARDRLIESFYWAVGVAFEPQYSDCRNSVAKMFSFVTIIDDIYDVYGTLDELELFTDAVERWDVNAINDLPDYMKLCFLALYNTINEIAYDNLKDKGENILPYLTKAWADLCNAFLQEAKWLYNKSTPTFDDYFGNAWKSSSGPLQLVFAYFAVVQNIKKEEIENLQKYHDTISRPSHIFRLCNDLASASAEIARGETANSVSCYMRTKGISEELATESVMNLIDETWKKMNKEKLGGSLFAKPFVETAINLARQSHCTYHNGDAHTSPDELTRKRVLSVITEPILPFERL

SEQ ID NO: 31 - protein, 185 aa, synthetic construct
MKSLEEKTIAKEQIFSGKVIDLYVEDVELPNGKASKREIVKHPGAVAVLAVTDEGKIIMVKQFRKPLERTIVEIPAGKLEKGEEPEYTALRELEEETGYTAKKLTKITAFYTSPGFADEIVHVFLAEELSVLEEKRELDEDEFVEVMEVTLEDALKLVESREVYDAKTAYAIQYLQLKEALQAQK

SEQ ID NO: 32 - DNA, 1509 bp, synthetic construct
  source 1..1509: mol_type = other DNA
atggttttaaccaataaaacagtcatttctggatcgaaagtcaaaagtttatcatctgcgcaatcgagctcatcaggaccttcatcatctagtgaggaagatgattcccgcgatattgaaagcttggataagaaaatacgtcctttagaagaattagaagcattattaagtagtggaaatacaaaacaattgaagaacaaagaggtcgctgccttggttattcacggtaagttacctttgtacgctttggagaaaaaattaggtgatactacgagagcggttgcggtacgtaggaaggctctttcaattttggcagaagctcctgtattagcatctgatcgtttaccatataaaaattatgactacgaccgcgtatttggcgcttgttgtgaaaatgttataggttacatgcctttgcccgttggtgttataggccccttggttatcgatggtacatcttatcatataccaatggcaactacagagggttgtttggtagcttctgccatgcgtggctgtaaggcaatcaatgctggcggtggtgcaacaactgttttaactaaggatggtatgacaagaggcccagtagtccgtttcccaactttgaaaagatctggtgcctgtaagatatggttagactcagaagagggacaaaacgcaattaaaaaagcttttaactctacatcaagatttgcacgtctgcaacatattcaaacttgtctagcaggagatttactcttcatgagatttagaacaactactggtgacgcaatgggtatgaatatgatttctaaaggtgtcgaatactcattaaagcaaatggtagaagagtatggctgggaagatatggaggttgtctccgtttctggtaactactgtaccgacaaaaaaccagctgccatcaactggatcgaaggtcgtggtaagagtgtcgtcgcagaagctactattcctggtgatgttgtcagaaaagtgttaaaaagtgatgtttccgcattggttgagttgaacattgctaagaatttggttggatctgcaatggctgggtctgttggtggatttaacgcacatgcagctaatttagtgacagctgttttcttggcattaggacaagatcctgcacaaaatgttgaaagttccaactgtataacattgatgaaagaagtggacggtgatttgagaatttccgtatccatgccatccatcgaagtaggtaccatcggtggtggtactgttctagaaccacaaggtgccatgttggacttattaggtgtaagaggcccgcatgctaccgctcctggtaccaacgcacgtcaattagcaagaatagttgcctgtgccgtcttggcaggtgaattatccttatgtgctgccctagcagccggccatttggttcaaagtcatatgacccacaacaggaaacctgctgaaccaacaaaacctaacaatttggacgccactgatataaatcgtttgaaagatgggtccgtcacctgcattaaatcctaa

SEQ ID NO: 33 - DNA, 1185 bp, synthetic construct
  source 1..1185: mol_type = other DNA
atgaagaactgtgtcatcgtgtcggccgtgcgtaccgcgatcggctcatttaacggcagcctggcctcgacctccgcgatcgatctgggcgcgacggtgatcaaagccgccatcgaacgcgcgaaaatcgactcgcagcacgtggatgaagtgattatgggcaacgtgctgcaggccggcctgggccagaatcccgcccgccaagccctgctgaaaagcgggctggccgaaaccgtctgtggcttcaccgtcaacaaggtatgtggttccggcctcaaatcggtcgctctggccgcccaagctatccaggcgggccaggctcagtcgattgtagcgggcggcatggaaaacatgtcgctggccccgtacctgctggacgccaaagcccgcagcggctaccgcctgggcgacggccaggtctatgatgtcatcctgcgggacggcctcatgtgcgcgacccatggctatcacatggggatcaccgccgagaacgtggcgaaagaatacggtattacccgcgaaatgcaggacgagctggcgctccactcgcagcggaaagcggcggcggcgatcgagagcggcgccttcacggccgaaatcgtgccggtgaacgtggtgacccgcaagaaaaccttcgtgttcagccaagacgaattcccgaaggccaactcgaccgcggaagcgctcggtgccctgcgccctgcattcgataaagcgggcaccgtgacggccgggaacgcaagcggcatcaacgatggcgcggcagcccttgtcatcatggaggaaagcgccgccctggcggccggcctgaccccactggcccgcatcaaatcgtatgcctcgggcggggtgccgccggccctgatgggcatggggcccgtcccggccacccagaaagccctgcagctggccggcctgcagttggccgacatcgatctgatcgaagccaacgaggcctttgccgcccagttcctggccgtgggcaaaaacctgggtttcgactccgagaaagtcaatgtgaacggcggcgccatcgcactgggccatccgatcggcgcctcgggcgcccgcattctggtcacgctgctccacgccatgcaggcgcgtgataaaaccctgggcctggccaccctgtgtatcggcgggggtcagggcatcgccatggtgatcgagcgcctgaactga

SEQ ID NO: 34 - DNA, 1476 bp, synthetic construct
  source 1..1476: mol_type = other DNA
atgaaactgtccacgaagctctgctggtgtggtatcaaaggccgcctgcgcccccagaaacagcagcagttgcataacaccaacctgcagatgaccgagctgaagaaacagaaaacggccgaacaaaaaacccgcccacagaacgtggggataaaaggcatccaaatttacattccgacccagtgcgtgaaccagtccgaactggagaaattcgatggcgtgtcccagggcaaatacaccatcgggctgggccaaaccaacatgtcattcgtgaacgaccgggaagacatctacagcatgagcctgaccgtgctgtccaaattgattaagtcctacaacatcgacaccaacaaaatcggtcggcttgaagtcggcacggaaaccctgatcgataaaagcaaaagcgtgaaatctgtgctgatgcagctcttcggcgaaaacacggatgtggaaggcatcgacacgctgaacgcctgctacggcggcactaacgcgctgttcaattcgctgaattggatcgagagcaacgcctgggacggccgggatgccatcgtggtctgtggcgatatcgccatctacgacaaaggcgccgcgcggccgaccggcggcgcgggcacggtggctatgtggatcggccccgacgcgccgatcgtcttcgattctgtccgcgccagctatatggaacacgcgtacgatttctacaaaccggatttcacgagcgaatatccgtacgtcgatggccatttttcgctgacgtgctatgtgaaggcgctggatcaagtgtataagagctactcgaagaaagcgatctcgaaaggcctggtgtcggatccggccggctccgacgcgctgaacgttctcaagtattttgactataacgtcttccacgtgccgacctgtaagctggtgaccaaaagctacggccgcctgctgtataacgatttccgggccaacccgcagctgttccccgaggtggacgccgaactggccacgcgggattatgatgagtccctcacggacaaaaacatcgaaaagaccttcgtcaacgtggccaagccgttccataaagaacgcgtggcgcagagtctgatcgtcccgaccaacaccggcaacatgtataccgccagtgtctacgctgcgttcgcgagcctgctgaattacgttggctcggatgacctgcagggcaagcgcgtgggcctgttcagctacggctcggggctggcggcgtccctgtactcctgcaagatcgtgggcgatgtccagcacatcatcaaggaactggatatcacgaacaaactggcgaagcgcatcacggaaacgccgaaagattatgaggccgcgatcgagctgcgcgaaaacgcgcacctgaaaaaaaatttcaaaccgcagggctcgatcgaacatctgcagtcgggggtgtattacctgaccaacatcgatgacaagttccgccgctcctatgacgtgaagaaataa

SEQ ID NO: 35 - DNA, 1509 bp, synthetic construct
  source 1..1509: mol_type = other DNA
atggttctgaccaacaaaacggttattagcgggagcaaagttaaatctttaagcagcgctcagtcatcgtcgtcagggccgagcagcagtagcgaggaagatgatagccgagatattgaaagtctagataagaaaattcgcccgctggaagaactggaagcgctgctgtcaagcggtaacaccaaacagctgaaaaacaaagaagtggcagcgcttgtcatccatggcaaactgcccctgtatgcgcttgaaaaaaaactgggcgatacaacccgcgcggtggcggtgcgccgcaaagcgctgtcaatccttgcggaagctccggtgctggcgagcgatcgactgccttataaaaactatgattatgatcgcgtgtttggcgcctgctgcgaaaacgttattggctatatgccgctcccggtgggcgtgattggcccgctggtgatagatggcaccagctatcatattccgatggcgaccaccgaaggctgcttagtcgcgagcgcaatgcgcggttgcaaagcaattaacgctggcggtggcgcgaccaccgtgcttaccaaagatggtatgacccgaggcccggtggttcgctttcccacccttaaacgctcaggcgcttgcaaaatttggctcgatagcgaagaagggcagaacgcgataaaaaaagcgttcaacagcaccagccgctttgcgcgcctgcagcatatccagacctgcctggcgggcgatctactgtttatgcgctttcgcaccactaccggcgatgctatgggcatgaacatgattagcaaaggcgtagaatatagcctgaaacagatggtggaagaatatggctgggaagatatggaagtcgttagcgtgagcggcaactattgcaccgataaaaaaccggcggcgatcaactggattgaaggccgtggcaaaagcgtggtggcggaagcgaccattccgggcgatgtggtgcgcaaagtgctaaaaagcgatgtgagcgcgctggtggaactgaatattgctaaaaacctggtgggcagcgcgatggcgggcagcgtgggcggctttaacgcccatgcggcgaatctggtgactgcggtgtttctggcgctgggccaggatccggcgcagaacgtggaaagcagcaactgcataaccctgatgaaagaagtcgatggcgatctgcgcattagcgtgagcatgccgagtatcgaagtgggcaccattggcggcggcaccgtgctggaaccgcagggcgcgatgctggatctgcttggcgtgcgcggtccgcatgcgaccgcgccgggcacgaacgcgcgccagctggcacgcattgtggcttgcgcggtactggcgggcgaattgagcctgtgcgcggcgttagcggcaggccatctggtgcagagccatatgacccataaccgcaaaccggcggaaccgaccaaaccgaacaacctggatgcgaccgatattaaccgactgaaagatggcagcgtgacctgcatcaagagctaa

SEQ ID NO: 36 - DNA, 1167 bp, synthetic construct
  source 1..1167: mol_type = other DNA
atgacaataggtatcgataaaataaacttttacgttccaaagtactatgtagacatggctaaattagcagaagcacgccaagtagacccaaacaaatttttaattggaattggtcaaactgaaatggctgttagtcctgtaaaccaagacatcgtttcaatgggcgctaacgctgctaaggacattataacagacgaagacaaaaagaaaattggtatggtaattgtggcaactgaatcagcagttgatgctgctaaagcagccgctgttcaaattcacaacttattaggtattcaaccttttgcacgctgctttgaaatgaaagaagcttgttatgctgcaacaccagcaattcaattagctaaagattatttagcaactagaccgaatgaaaaagtattagttattgctacagatacagcacgttatggattgaattcaggcggcgagccaacacaaggtgctggcgcagttgcgatggttattgcacataatccaagcattttggcattaaatgaagatgctgttgcttacactgaagacgtttatgatttctggcgtccaactggacataaatatccattagttgatggtgcattatctaaagatgcttatatccgctcattccaacaaagctggaatgaatacgcaaaacgtcaaggtaagtcgctagctgacttcgcatctctatgcttccatgttccatttacaaaaatgggtaaaaaggcattagagtcaatcattgataacgctgatgaaacaactcaagagcgtttacgttcaggatatgaagatgctgtagattataaccgttatgtcggtaatatttatactggatcattatatttaagcctaatatcattacttgaaaatcgagatttacaagctggtgaaacaatcggtttattcagttatggctcaggttcagttggtgaattttatagtgcgacattagttgaaggctacaaagatcatttagatcaagctgcacataaagcattattaaataaccgtactgaagtatctgttgatgcatatgaaacattcttcaaacgttttgatgacgttgaatttgacgaagaacaagatgctgttcatgaagatcgtcatattttctacttatcaaatattgaaaataacgttcgtgaatatcacagaccagagtag

SEQ ID NO: 37 - DNA, 1278 bp, synthetic construct
  source 1..1278: mol_type = other DNA
atgcaaaatttagataagaattttcgacatttatctcgtaaagaaaagttacaacaattggttgataagcaatggttatcagaagaacaattcgacattttactgaatcatccattaatcgatgaagaagtagccaatagtttaattgaaaatgtcatcgcgcaaggtgcattacccgttggattattaccgaatatcattgtggacgataaggcatatgttgtacctatgatggtggaagagccttcagttgtcgctgcagctagttatggtgcaaagctagtgaatcagactggcggatttaaaacggtatcttctgaacgtattatgataggtcaaatcgtctttgatggcgttgacgatactgaaaaattatcagcagacattaaagctttagaaaagcaaattcataaaattgcggatgaggcatatccttctattaaagcgcgtggtggtggttaccaacgtatagcgattgatacatttcctgagcaacagttactatctttaaaagtatttgttgatacgaaagatgctatgggcgctaatatgcttaatacgattttagaggccataactgcatttttaaaaaatgaatttccgcaaagcgacattttaatgagtattttatccaatcatgcaacagcgtccgttgttaaagttcaaggcgaaattgatgttaaagatttagcaaggggcgagagaactggagaagaggttgccaaacgaatggaacgtgcttctgtattggcccaagtagatattcatcgtgcagcaacacataataaaggtgttatgaatggcatacatgctgttgttttagcaacaggaaatgatacgcgtggtgcagaagcaagtgcgcatgcatacgcgagtcgtgacggacagtatcgtggtattgctacatggcgttacgatcaagatcgtcaacgattgattggtacaattgaagtgcctatgacattggcaatcgttggcggtggtacaaaagtattaccaattgctaaagcttcattagagctactaaatgtagagtcagcacaagaattaggtcatgtagttgctgccgttggtttagcgcaaaactttgcagcatgtcgcgcgcttgtgtcagaaggtattcaacaaggtcatatgagtttacaatataaatcattagctatcgttgtaggggcaaaaggtgatgaaattgctaaagtagctgaagctttgaaaaaagaaccccgtgcaaatacacaagcagcggaacatattttacaagaaattagacaacaatag

SEQ ID NO: 38 - DNA, 1152 bp, synthetic construct
  source 1..1152: mol_type = other DNA
atgacaattgggattgataaaattagtttttttgtgcccccttattatattgatatgacggcactggctgaagccagaaatgtagaccctggaaaatttcatattggtattgggcaagaccaaatggcggtgaacccaatcagccaagatattgtgacatttgcagccaatgccgcagaagcgatcttgaccaaagaagataaagaggccattgatatggtgattgtcgggactgagtccagtatcgatgagtcaaaagcggccgcagttgtcttacatcgtttaatggggattcaacctttcgctcgctctttcgaaatcaaggaagcttgttacggagcaacagcaggcttacagttagctaagaatcacgtagccttacatccagataaaaaagtcttggttgtagcagcagatattgcaaaatatggattaaattctggcggtgagcctacacaaggagctggggcggttgcaatgttagttgctagtgaaccgcgcatcttggctttaaaagaggataatgtgatgctgacgcaagatatctatgacttttggcgtccaacaggccatccgtatcctatggtcgatggtcctttgtcaaacgaaacctacatccaatcttttgcccaagtctgggatgaacataaaaaaagaaccggtcttgattttgcagattatgatgctttagcgttccatattccttacacaaaaatgggcaaaaaagccttattagcaaaaatctccgaccaaactgaagcagaacaggaacgaattttagcccgttatgaagaaagcatcatctatagtcgtcgcgtaggaaacttgtatacgggttcactttatctgggactcatttcccttttagaaaatgcaacgactttaaccgcaggcaatcaaattgggttattcagttatggttctggtgctgtcgctgaatttttcactggtgaattagtagctggttatcaaaatcatttacaaaaagaaactcatttagcactgctagataatcggacagaactttctatcgctgaatatgaagccatgtttgcagaaactttagacacagatattgatcaaacgttagaagatgaattaaaatatagtatttctgctattaataataccgttcgctcttatcgaaactaa

SEQ ID NO: 39 - DNA, 2412 bp, synthetic construct
  source 1..2412: mol_type = other DNA
atgaaaacagtagttattattgatgcattacgaacaccaattggaaaatataaaggcagcttaagtcaagtaagtgccgtagacttaggaacacatgttacaacacaacttttaaaaagacattccactatttctgaagaaattgatcaagtaatctttggaaatgttttacaagctggaaatggccaaaatcccgcacgacaaatagcaataaacagcggtttatctcatgaaattcccgcaatgacagttaatgaggtctgcggatcaggaatgaaggccgttattttggcgaaacaattgattcaattaggagaagcggaagttttaattgctggcgggattgagaatatgtcccaagcacctaaattacaacgatttaattacgaaacagaaagctatgatgcgcctttttctagtatgatgtacgatgggttaacggatgcctttagtggtcaagcaatgggcttaactgctgaaaatgtggccgaaaagtatcatgtaactagagaagagcaagatcaattttctgtacattcacaattaaaagcagctcaagcacaagcagaagggatattcgctgacgaaatagccccattagaagtatcaggaacgcttgtggagaaagatgaagggattcgccctaattcgagcgttgagaagctaggaacgcttaaaacagtttttaaagaagacggtactgtaacagcagggaatgcatcaaccattaatgatggggcttctgctttgattattgcttcacaagaatatgccgaagcacacggtcttccttatttagctattattcgagacagtgtggaagtcggtattgatccagcctatatgggaatttcgccgattaaagccattcaaaaactgttagcgcgcaatcaacttactacggaagaaattgatctgtatgaaatcaacgaagcatttgcagcaacttcaatcgtggtccaaagagaactggctttaccagaggaaaaggtcaacatttatggtggcggtatttcattaggtcatgcgattggtgccacaggtgctcgtttattaacgagtttaagttatcaattaaatcaaaaagaaaagaaatatggagtggcttctttatgtatcggcggtggcttaggactcgctatgctactagagagacctcagcaaaaaaaaaacagccgattttatcaaatgagtcctgaggaacgcctggcttctcttcttaatgaaggccagatttctgctgatacaaaaaaagaatttgaaaatacggctttatcttcgcagattgccaatcatatgattgaaaatcaaatcagtgaaacagaagtgccgatgggcgttggcttacatttaacagtggacgaaactgattatttggtaccaatggcgacagaagagccctcagtgattgcggctttgagtaatggtgcaaaaatagcacaaggatttaaaacagtgaatcaacaacgtttaatgcgtggacaaatcgttttttacgatgttgcagacgccgagtcattgattgatgaactacaagtaagagaaacggaaatttttcaacaagcagagttaagttatccatctatcgttaaacgcggcggcggcttaagagatttgcaatatcgtgcttttgatgaatcatttgtatctgtcgactttttagtagatgttaaggatgcaatgggggcaaatatcgttaacgctatgttggaaggtgtggccgagttgttccgtgaatggtttgcggagcaaaagattttattcagtattttaagtaattatgccacggagtcggttgttacgatgaaaacggctattccagtttcacgtttaagtaaggggagcaatggccgggaaattgctgaaaaaattgttttagcttcacgctatgcttcattagatccttatcgggcagtcacgcataacaaagggatcatgaatggcattgaagctgtcgttttagctacaggaaatgatacacgcgctgttagcgcttcttgtcatgcttttgcggtgaaggaaggtcgctaccaaggtttgactagttggacgctggatggcgaacaactaattggtgaaatttcagttccgcttgcgttagccacggttggcggtgccacaaaagtcttacctaaatctcaagcagctgctgatttgttagcagtgacggatgcaaaagaactaagtcgagtagtagcggctgttggtttggcacaaaatttagcggcgttacgggccttagtctctgaaggaattcaaaaaggacacatggctctacaagcacgttctttagcgatgacggtcggagctactggtaaagaagttgaggcagtcgctcaacaattaaaacgtcaaaaaacgatgaaccaagaccgagccttggctattttaaatgatttaagaaaacaataa

SEQ ID NO: 40 - DNA, 894 bp, synthetic construct
  source 1..894: mol_type = other DNA
atggaattcgatttcaacaaatatatggactcaaaggccatgactgtgaatgaagctctaaataaggccataccgctgcgctacccgcaaaagatctacgaatccatgcgttatagcctcctcgcgggcggcaaacgcgtacgtccggtcctgtgtattgcggcctgtgaactggtgggcgggaccgaagaactggcgatccccacggcctgcgctatcgagatgatccataccatgagcctgatgcatgatgatcttccgtgcatcgacaacgacgacctgcgcagggggaagcctaccaaccataagattttcggcgaagacactgccgttacagccggtaacgcgctgcatagctacgccttcgagcatattgccgtgagtacctccaagactgttggcgccgatcgtattctgcgcatggtgtcggaactgggccgcgcaacgggcagtgagggcgttatggggggccagatggtggacatcgcctccgaaggtgatccgtctattgaccttcaaaccctggaatggatccacatccataaaaccgcgatgttgctggaatgcagcgtggtatgtggcgcgatcatcggcggcgcgtccgaaatagtgatcgaacgcgcccgacgctatgcgcgttgcgtgggcctactgttccaagtggtcgatgacatcctggacgtgacgaagagcagcgatgaactgggcaaaacagcgggcaaagacctgatctcggacaaagcgacgtatccaaaattaatgggcctcgagaaagccaaggaattctcggatgagctgctgaatcgcgccaagggcgagctgtcgtgctttgatccagtgaaagcggcaccgctgctgggactggcagactacgtagcgttccggcagaactga

SEQ ID NO: 41 - DNA, 990 bp, synthetic construct
  source 1..990: mol_type = other DNA
atgcaagaattcgagtttgccgttcctgctccgtcacgagtctcacctgatctggcacgcgcgcgagctcgtcatctcgactgggtccacgccatggacctggtgcgcggcgaagaggccaggcggcgttacgagttctcctgtgtagcggacatcggtgcctatggatatccgcatgcgaccggtgcggatctggatctctgcgtcgacgtcctcgggtggactttcctctttgatgatcagttcgacgccggagacgggcgggaacgggatgctttggctgtctgcgcggagcttacggatttattgtggaaagggacggcggctacagcagcgtcgccgccaatcgtcgtggcattcagcgattgctgggagcggatgcgggctggaatgtccgacgcgtggcggcggcggacggtccatgagtgggtggattatctggctggctggcccaccaagctggcggaccgcgctcacggcgccgtcctggacccggccgctcatctgagagcgcggcatcggacgatctgctgccggccgctcttcgccctggcggaacgagtcggggggtacgaggtcccgcggcgtgcatggcactcctcgcggctcgacgggatgcggttcaccacgtccgatgcggtgatcggcatgaatgagctccactcgttcgagaaagaccgggcgcagggacacgccaacttagtcctgagcttggtgcaccacgggggattaaccggaccggaggccgtcacccgggtgtgcgacctagtccagggctcgatcgagtccttcctgcgactgcgctccgggctgccggagttgggcagagccctcggtgtcgaaggggccgtgctcgaccggtacgccgacgcgctgtccgcgttttgccgcgggtatcacgactggggtcgtggcgcttctcggtacaccacccgagatcaccccggcgatctcggactggagaacctcgtcgctcggagttcgggctga

SEQ ID NO: 42 - DNA, 1788 bp, synthetic construct
  source 1..1788: mol_type = other DNA
atggctaccgaactcctgtgtctgcatcgcccgatctccctcacgcataaactgtttcgcaacccgctcccgaaggtgattcaggcgacgccgctgacgctgaaactgcgctgctcggtcagcaccgaaaacgtgagcttcaccgagaccgagaccgaggcgcgccgtagcgcgaattatgagccgaactcctgggactacgattacctgctgtctagcgacaccgacgagagcatcgaagtctataaagacaaagcgaaaaagttggaggccgaggtgcgccgcgaaatcaacaacgagaaagcggaattcctgaccctcctggaactgatcgacaacgtgcaacgcctgggcctgggctataggttcgaaagcgatatccgcggcgcgctggaccgttttgtttcttcgggcggcttcgacgcggtgaccaaaaccagcctccacggcaccgcgctgagctttcgcctgctgcgccaacacggcttcgaagtcagccaggaagccttcagcggcttcaaggaccagaacggcaactttctggagaacctgaaagaggatatcaaagcgattctgtccctgtacgaggcgagcttcctggccctggaaggcgaaaacatcctggacgaggccaaagtcttcgcgatcagccacctgaaagaactgtctgaagaaaaaatcgggaaggaactggccgagcaggtgaaccatgcgctggaactgccgctgcaccgccgtacccagcgtctggaggcagtgtggtcgatcgaggcgtatcggaaaaaagaagatgcgaatcaagtgctgctggaactggccatcctggactacaacatgatccagtcggtgtaccagcgcgatctgcgcgaaacgagcagatggtggcggcgcgtgggcctggcgaccaaactccatttcgcgcgcgaccgtctgatcgaaagcttctattgggcggtgggcgtggccttcgagccgcagtattccgattgccgtaattccgtggccaaaatgttctcgttcgtaaccatcattgatgatatctatgatgtgtacggcaccctggacgagctggaactgttcaccgacgccgtggaacgctgggacgtgaacgccatcaacgatctgccggactacatgaaactgtgctttctggccctgtataataccatcaacgagatcgcctacgataacctgaaggacaaaggcgagaatattctgccgtatctgacgaaagcgtgggccgacctgtgtaatgccttcctgcaagaggcgaagtggctgtacaacaagagcaccccgacctttgatgattactttggcaacgcatggaagtctagctcgggcccgctgcagctggtgttcgcgtacttcgcagttgtgcagaatatcaagaaagaagaaattgaaaacctgcaaaaataccatgacaccatcagccgcccctcgcacattttccgcctgtgtaacgatctggcgagcgcgagcgcggaaattgcgcgcggcgaaacggccaactccgtgtcctgctatatgcgtacgaaagggatcagcgaggaactcgccaccgagtcggttatgaacctgatcgatgagacctggaaaaagatgaacaaggaaaagctgggcggcagcctctttgccaagccgttcgtggaaacggccatcaatctggcgcggcaaagccattgtacctaccataacggcgatgcgcacacctccccggacgaactgacccgcaaacgcgtgctgagcgttatcacggaaccgatcctgccgttcgaacggtga

SEQ ID NO: 43 - DNA, 558 bp, synthetic construct
  source 1..558: mol_type = other DNA
atgaaaagcctggaggaaaagaccatcgcgaaagagcagatcttctcgggcaaggtgatcgacctgtacgtggaggacgtggagctgccgaacggcaaagcgtcgaaacgcgaaattgtgaaacaccccggggcggtggccgtgctggcggtgaccgatgaaggcaaaatcattatggtgaaacagtttcgcaagccgctggaacgcaccatcgtggaaatcccagccggtaagctcgaaaaaggagaagaaccggagtacacggcgctgcgcgaactggaagaggaaaccggctataccgccaaaaagctcaccaagattaccgcgttttacacctcgccgggcttcgccgatgagatcgtgcatgtgttcctggcggaagagctgtcggtgctggaggaaaaacgcgaactggacgaagatgagttcgtggaagtgatggaggtgaccctggaagatgcgctgaaactggtggagagccgcgaagtgtacgacgccaagaccgcttacgcgattcagtacctgcagctgaaagaggccctgcaagcgcagaaatga

SEQ ID NO: 44 - DNA, 993 bp, synthetic construct
  source 1..993: mol_type = other DNA
atgccggcgggccatgaagaatttgacatcccgttcccttcgcgagtcaaccctttccatgctcgcgctgaggaccgtcacgttgcctggatgagggcaatggggctgatcacaggcgatgccgctgaagctacctaccgcagatggagcccggctaaagtgggggcgagatggttttatctcgcgcagggagaagacctggatctgggctgtgatatattcggttggttcttcgcttatgatgaccattttgacggaccgactggaactgatccgcgccagacagcagcgtttgtgaatcgcaccgtagcgatgctggatccgcgcgcggaccctactggagagcatccactgaatatcgcctttcatgatttatggcagcgagaaagcgcaccgatgtccccgctctggcaacgccgcgccgtcgatcattggacccaatatctgaccgctcatattacggaagctactaacagaacccgtcacacttcgccgaccatcgccgattacctagagcttcgccatcgcactggattcatgccgccgctacttgacttaattgaacgagtctggcgagccgaaatcccggctcccgtctacacgacgccggaggtgcagactcttctgcacacgaccaatcagaacattaatatcgtcaacgacgtattatccctggaaaaagaagaagcgcacggagatccgcataatctggttctggtgattcaacatgaacgtcagagtacacgccaacaggcgcttgccaccgcacgccgaatgattgatgaatggaccgatacgtttattcgaacggaaccgaggctgccggctctctgcggccgcctgggcattcctttagctgaccgcacgagcctttacacagctgttgaaggcatgcgagctgcgattcgtggaaactatgattggtgcgcggaaacgaatcgatatgccgtgcatcgcccgacaggcacgggtcgcgcaaccaccccgtggtga

SEQ ID NO: 45 - DNA, 1887 bp, synthetic construct
  source 1..1887: mol_type = other DNA
atggccctggtgagcgccgtcccgctgaactcgaaactgtgcctgcggcgcaccctgtttggcttctcgcatgaactgaaagcaatccactcgaccgtcccgaacctgggcatgtgccgcggcggtaaatcgatcgcgccgagcatgagcatgtcgagcaccacctcggtctcgaatgaagacggcgtgcctcggcggatcgcgggccaccatagcaacctgtgggacgatgattccattgcgtcgttgtcgacgtcgtacgaagccccaagctatcgcaagcgtgccgataagttaattggcgaagtgaaaaatattttcgacctgatgtcggtggaagatggggtcttcaccagcccgctgagcgatttgcatcatcgcctgtggatggtggattcggtagagcgtctgggcatcgatcgtcatttcaaagatgaaatcaattctgccctggaccatgtctattcgtactggaccgaaaagggcattggccgcggtcgcgaatcgggggtgacggacctgaacagcaccgccctgggcctgcgcaccctgcggctgcacggctacaccgtgtcgagccacgttctggatcatttcaaaaacgagaagggccaattcacctgtagcgcgattcagaccgaaggcgagattcgcgacgtcctgaatctgttccgcgcgtcgctgattgcgttccccggcgaaaaaatcatggaagcggcggaaatcttctctaccatgtacctcaaagatgccctgcagaaaatccctccgagcggcctgagccaggagattgagtacctgctggaattcgggtggcataccaacttaccgcgtatggaaacccgcatgtacatcgatgtgtttggcgaggacaccacatttgaaaccccctatctgattcgcgaaaaactgctggagctggccaaactggaatttaacatcttccacagcctggtgaaacgggagctgcagagcctgagccgctggtggaaagactatggcttcccggaaatcacgttctcccgccaccgtcatgtcgaatactacaccctggccgcctgtatcgcgaacgacccgaaacatagcgcgttccgcctgggcttcggcaaaatcagccatatgattaccattctggatgatatttatgatacgttcggcaccatggaagagctgaagctgctcaccgccgccttcaaacgctgggatccctcctcgatcgagtgcttgccagattacatgaagggcgtgtacatggcggtgtatgacaacatcaacgagatggcccgcgaggcgcaaaaaatccagggctgggataccgtgtcgtatgcgcgcaagtcgtgggaagccttcatcggcgcatacatccaggaagccaagtggatcagcagcggctacctgccgacctttgatgaatatctcgaaaacggcaaagtctcgtttggctcccgcatcacgaccctggagccgatgctgacgctgggcttcccgctgccgccgcgaatcctgcaggagatcgatttcccgagcaaattcaacgatttgatctgtgcgatcctgcgcctgaagggcgacacgcagtgctataaagcggatcgcgcgcggggcgaggaagccagcgccgttagctgttatatgaaagatcaccctggcatcaccgaagaagatgccgtcaaccaggtcaacgccatggtcgataacctgaccaaagaactgaattgggagctgctgcgcccggacagcggcgtgccgatcagctataagaaggtcgcgtttgacatctgtcgcgtgtttcattatggctacaaataccgcgacggcttctccgtggcgtccattgaaatcaaaaacctcgtgacccgtaccgtggtggagaccgtgccgctgtga

SEQ ID NO: 46 - DNA, 1887 bp, synthetic construct
  source 1..1887: mol_type = other DNA
atggccctggtatccaccgccccgctggcgagcaaatcgtgtctgcataaatcactgatttcgtcgacccatgaactgaaggcgctgtcgcgtaccatcccagccctgggcatgagccgacgtggtaaaagcattacgccgtcgatctccatgagcagcaccacggtggtgaccgacgatggcgtgcgccgccgcatgggcgacttccattcgaacctgtgggacgatgatgtgatccagtcgctgccaaccgcctatgaagagaagtcgtatttggaacgggccgaaaagctgatcggggaagtcaagaacatgtttaattcgatgagcctggaagatggcgagctgatgtcgccgttgaacgatctgatccaacgcctgtggattgttgactccctcgaacggctgggcatccatcgccatttcaaagatgagatcaaatcggccctggattatgtctactcctattggggcgaaaacggcattggctgcggccgggagtcggtggtgaccgatctgaactctacggccctgggcctgcgcacgttgcggctgcatggctatccggtgtcgtcggacgtgtttaaagcgttcaaaggccagaacggccaattcagctgctcggagaacattcagaccgacgaggaaatccgcggcgtgctgaatctgtttcgcgcctctctgatcgccttcccgggagaaaagatcatggacgaagcggaaatcttcagcaccaagtatctcaaagaagccctgcagaaaattccggtgtcgtccctgagccgcgaaatcggtgacgtgctggagtatggctggcatacctatctgccgcgcctggaagcccgcaactacatccaggtctttggccaggacacggaaaacacgaaaagctacgtgaagagcaaaaaactgctggaactggccaagctggaattcaatatcttccagtcgctgcagaaacgcgaactggaaagcctggtgcggtggtggaaagaatcgggcttcccagaaatgaccttctgtcgccaccgccatgtcgaatactacaccctggcctcttgtattgcattcgaaccgcagcattccggcttccgtctgggcttcgcgaaaacctgtcacctgatcaccgtcctggatgacatgtacgacacctttggcaccgtagatgaactggagctgtttaccgcgacgatgaagcgctgggacccgagttcgatcgactgtctgcccgaatacatgaagggcgtctacatcgcggtgtatgacaccgtgaacgagatggcgcgggaggccgaagaggcccagggccgcgataccctgacgtacgcccgcgaggcctgggaagcctatattgattcgtacatgcaagaagcccggtggatcgcgaccggctacctgccgtcgtttgatgaatactacgagaacggcaaagtgagctgtggccatcgcataagtgccctgcagccgatcctgaccatggacatcccgtttccggaccatatcctgaaagaagtggattttcccagcaagctgaatgatctggcctgcgccatcctgcggctgcgtggcgacacccgctgctataaagcggatcgcgcgcgcggcgaggaggcgagcagcatcagctgctatatgaaagacaacccgggcgtgtccgaagaagatgccctggaccacatcaatgcgatgatctcggatgtgatcaaaggcttgaactgggaactgctgaaaccggatatcaacgtaccgatttcggcgaagaaacacgcgttcgatatcgcccgcgccttccactacggctataagtatcgcgatggctatagcgtagcgaacgtggaaactaaaagcctggtcacccgcaccctgctggaaagcgtcccgctgtga

SEQ ID NO: 47 - DNA, 1629 bp, synthetic construct
  source 1..1629: mol_type = other DNA
atgcgccgcagcggcaattatcaagcgccggtttggaacaacgatttcatccagtccttctccacggacaaatacaaagacgagaaattcctgaagaaaaaagaggaactgatcgcgcaagtgaaggtcttgctgaacaccaaaatggaggccgtgaagcagctggagctgatcgaggatctgcgcaacctgggcctcacctactatttcgaagacgagtttaaaaagatcctgacgtcgatctacaacgaacacaaaggcttcaagaacgaacaagtgggcgatctgtacttcaccagcctggcgttccggctgctgcgcctgcatggcttcgatgtctcggaggacgtcttcaacttcttcaaaaacgaagacggcagcgactttaaagcgagtctgggggagaacaccaaggacgtgctcgagctgtacgaagccagtttcctcatccgcgtgggcgaggtgaccctggagcaggcccgcgtctttagcaccaagattctggagaaaaaggtggaggagggcatcaaagacgagaagctgctggcgtggattcagcattcgctggccctgccactgcattggcgcatccagcggctggaagcccgctggttcctggatgcgtataaagcccgcaaggatatgaacccgatcatctacgaactgggcaagatcgacttccatattatccaggagacgcagctgcaggaagttcaagaggtgagccagtggtggacgaataccaacctggccgaaaagctgcctttcgtccgggatcgaatcgtcgaatgttacttctgggcgctgggtctgtttgagccgcatgagtatggctaccaacgcaagatggccgccattatcatcaccttcgtgacgattatcgatgatgtctatgatgtctatgacaccctggacgaattgcaattattcaccgacgcgatccgcaagtgggacgtcgaatcgatctcgaccctgccgtattatatgcaggtctgctacctcgcggttttcacttacgcgagcgaactggcttatgacatcctgaaggatcaaggcttcaactcgatcagctacctgcagcgttcctggctgagcctggtggagggcttcttccaggaagcgaaatggtattatgccggctacaccccgaccctggcggagtacctcgaaaacgcgaaagtgagcatctcctcgccgacgatcatctcccaggtgtatttcaccctcccgaatagcaccgagcgcaccgtggtcgaaaatgtcttcggttaccacaacattctgtacctgtctggcatgatcctgcggctggccgatgacctggggaccacccagttcgaactgaaacggggcgacgttcaaaaagcgatccagtgctacatgaacgacaacaacgccacagaagaggagggcacggaacacgtgaaatacctgctgcgcgaagcgtggcaggaaatgaacagcgcaatggcggacccggattgcccactgtccgaggacctggtttttgcggcggcgaacctgggccgcacctcccagttcatctacctggatggcgatgggcacggcgtgcaacattcggaaattcacaaccagatgggtggcctcatcttcgaaccgtacgtttga

SEQ ID NO: 48 - DNA, 1704 bp, synthetic construct
  source 1..1704: mol_type = other DNA
atgagctgcgcccggatcacggttactcttccttatcggtcggcaaagacgagcattcagcgtggcatcacccattatccggccctgattcgcccgcgcttcagcgcgtgcaccccgctggcctcggctatgccgctgtcgtcgaccccgctgattaacggggataactcgcaacgcaaaaacacgcgtcagcatatggaagaaagtagcagcaaacgccgcgaatacctgctggaggagaccacccggaaactgcagcgcaacgacacggagtccgtggaaaagcttaagctcatcgacaacatccagcagctgggcattggctattatttcgaggacgcgattaatgccgtgctgcgttccccgttcagcaccggcgaagaagacctctttaccgccgccctgcgcttccgccttctgaggcataatggcatcgaaatcagcccggaaattttcctgaaattcaaagacgagcgcggcaagttcgatgagagcgacacgctggggctgctgtctctgtacgaagccagcaacctgggcgtcgccggcgaggaaatcctggaggaggcgatggaattcgcagaggcgcgcctgcggcgcagcctgtccgagcccgccgcaccgctccatggcgaagtcgcgcaggccctggatgtgccgcgtcacctgcgtatggcccgtctggaagctcgccgtttcatcgagcaatatggcaagcagagcgatcacgacggagatctgctggaactggcgatcttagattataaccaggtgcaagcccagcatcagagcgaactgaccgagatcatccgctggtggaaggagctgggcctggtcgataaactgagttttggacgtgatcgccctctggagtgctttctgtggaccgttggcctgctgccggaaccaaagtattcgagcgtgcgcatcgaactggcgaaagccatctcgatcctgcttgtcatcgatgatatcttcgacacctatggtgagatggatgatctcatccttttcaccgacgcgatccgtcgctgggacctggaagcgatggaaggcctgccggaatatatgaaaatctgctatatggcgctgtacaacaccactaacgaggtgtgctacaaggtgctgcgtgataccggccgcatcgtgctgctgaacctgaaaagcacctggattgacatgatcgaagggttcatggaagaagcgaaatggttcaacggcggttcggccccgaaactggaggaatacatcgaaaacggcgtctcgaccgcgggcgcgtacatggctttcgctcatattttcttcctgatcggcgaaggcgtgacgcaccagaacagccagctcttcactcagaaaccgtatccgaaagtcttttcggccgccggccgcatcctccgcctgtgggatgatctgggtaccgccaaagaagagcaggaacgtggggatctagccagttgcgtccagctgtttatgaaagaaaagtcactgaccgaagaagaggcgcgcagcagaattctcgaagaaatcaagggcctgtggagagacctgaacggcgaactggtgtacaacaaaaacctgcccttgagcatcatcaaggtggccctgaacatggcccgagcgtcgcaggtcgtatacaaacatgatcaagatacgtacttcagctcggtggacaactatgtggacgcactgttctttacccagtga

SEQ ID NO: 49 - DNA, 1821 bp, synthetic construct
  source 1..1821: mol_type = other DNA
atgagcagctgcattaatccgagcaccctggtcacgagcgtgaacgccttcaaatgcctgccgctggcgaccaacaaagctgcgattcgcattatggccaaatacaagccggtgcaatgtctgatcagcgcgaagtacgacaacctcacggtggatcgccggtcggctaactaccagccctccatctgggatcatgacttcttgcagtcacttaactccaactacaccgacgaagcgtacaagcgccgcgcggaggagctgcgcggcaaagtgaaaatcgcgatcaaagacgtgatagagccgctggaccaactggaactgatcgataacctgcagcgcctcggcctggcgcatcgcttcgagaccgaaatccgcaacattctgaacaacatctacaacaataacaaagattacaactggcgcaaggagaacctgtacgcgacctcgctggagttccgcctgctgcgccagcatggctacccggtgtcgcaggaggtgtttaacgggttcaaggacgatcagggcgggttcatttgtgatgacttcaaaggcatcctgagcctgcatgaggccagctactactccctggaaggcgaatctattatggaagaggcctggcaattcacctcgaagcacctgaaggaggtgatgatttcgaagaacatggaggaggacgtgtttgtggcggagcaggccaaacgtgcgctggagctgccgctgcactggaaagtgccgatgctggaggcccggtggttcatccatatctacgagcgccgggaggataaaaatcacttgctgctggaactggccaagatggagttcaacacgctgcaggccatctaccaggaggaactgaaagaaatcagcggttggtggaaagataccggcctgggcgagaaactcagctttgcgcgcaaccgtctggtcgcctcgtttctgtggtcgatgggcatcgccttcgagccgcagttcgcttactgccgccgcgtgctgacgatctcgatcgcgctgatcaccgttatcgatgacatctacgatgtgtatggcaccctggatgaactggagatcttcaccgacgccgtggaacgctgggatataaattacgccctgaaacacctgccgggctacatgaagatgtgctttctggcgttgtacaacttcgtgaacgaattcgcttactacgtcctgaaacagcaagatttcgacctgctgctgagcattaaaaacgcgtggctcggcctgatccaagcctacctggtcgaggcgaaatggtaccatagcaaatatacgccgaaactcgaagagtacctggaaaacggcctggtgtcgatcacaggcccgctgatcatcaccatctcgtatctctcgggcaccaatccgatcatcaaaaaagaactggaattcctggaaagcaacccagacatcgtgcattggtcgtccaagatcttccgactgcaggacgatctgggcacgtcctccgacgagatccagcgcggcgacgtgccgaaatccatccagtgttatatgcatgagaccggcgcgagcgaagaggtggcccgccagcacatcaaagatatgatgcgccagatgtggaaaaaggttaacgcctacaccgcggataaagacagcccgctgaccggcacgactaccgaattcctgctcaacctggtgcgcatgtcccacttcatgtatctgcatggcgacgggcacggcgtgcagaaccaggaaaccattgacgtgggcttcacgctgctgttccagcctattccgcttgaagataaacatatggcgttcactgcttccccgggcactaaaggctga

SEQ ID NO: 50 - DNA, 1800 bp, synthetic construct
  source 1..1800: mol_type = other DNA
atggccctgaaagtgctgagcgtggcgacccagatggcgatcccgtccaacctgaccacctgcctgcagccgtcgcatttcaaaagctcgccaaaactgctgtcctcgaccaacagctccagccggtcgcgcctgcgcgtgtactgttcgtcctcccagctgaccacggagcgccggtccggcaactataacccgagccggtgggatgtgaacttcattcagagcctgctgagcgactataaagaagacaaacacgtcatccgcgcgtcggaactggtgaccctggtgaagatggagctcgagaaggaaacggaccagattcgccagctggaactgatcgatgatctgcagcggatgggcctgtccgaccactttcaaaatgagtttaaagaaatcctgtccagtatctatctggaccaccattactacaagaacccgtttccgaaagaggagcgtgatctgtatagcacctcgttggccttccgcctgctgcgcgagcacggctttcaggtagcccaggaagtgttcgatagctttaaaaatgaagaaggcgagttcaaggagagcctgtcggatgacacccggggcctgctgcagttgtacgaagccagcttcctgctgaccgaaggcgagaccacgctggaatctgcgcgcgagtttgccaccaaatttctggaagaaaaggtcaacgaagggggcgtggatggcgatctgctgacccgcatcgcctattccctggacatcccgctgcattggcgcatcaagcgcccgaacgccccggtgtggatcgaatggtaccgcaaacgcccggatatgaacccggtggtcttagagctggccatcctggacctgaacatcgtccaggcgcagttccaagaggagctgaaagaaagcttccgctggtggcgcaacaccggcttcgtggagaaattgccgttcgcgcgcgatcgcttggttgaatgctatttctggaataccggcatcatcgagccgcgccagcatgcgagcgcccgcattatgatgggcaaagtgaatgcgctgatcaccgtgatcgacgatatctacgacgtctatggcaccctggaggaactggagcagttcaccgacctgatccgacgctgggatatcaacagcatcgaccaactgccggattatatgcagctgtgctttcttgcgctcaacaacttcgtggacgacaccagctacgatgtcatgaaagaaaaaggcgtgaacgtgatcccgtacctgcgccagtcgtgggtggacctggccgataaatatatggtggaagcgcgctggttttacggggggcataagccgagcctcgaggaatacctggagaacagctggcagtcgattagcggcccgtgcatgctgacccatattttttttcgcgtgactgacagcttcacgaaggagaccgtggatagcctgtacaaataccacgacctggtccgctggagctcgtttgtgctgcgcctggcggatgatctgggcacctcggtcgaggaggtgtcgcggggcgacgtgccgaaaagcctgcaatgttacatgtcggactataacgcgagcgaagcagaagcccgcaaacatgtgaaatggttaattgcggaagtgtggaaaaagatgaacgcggagcgcgtgagcaaggactccccgtttggcaaagacttcatcggctgcgcggtggatctgggccgcatggcccagctgatgtatcataacggcgatggccatggcacgcagcatcctatcattcatcagcaaatgacccgcaccctgttcgaacccttcgcctga

SEQ ID NO: 51 - DNA, 1890 bp, synthetic construct
  source 1..1890: mol_type = other DNA
atggccgtgcaccagttcagcccgtcgattgtctcgagcctgccgacgatctcgacctataacaacaatcacttctgtcggttcttcaccccgaaaacctcgatcagcccgatctcgaagaccaaaagcaaatcgtccacctgttaccccatccagtgcaccgtggtgaataacagctcgcccagctcgacgatcgtgcgccgctccgcgaactatgaaccgcctatctggagcttcgactacatccagagcttgtcgacccagtacaagggcgaatcctatacccgccagctgaacaaactgaagaaagaggtgaagcggatgctgctgcgcatggaaatcaatagcctggccttgctggagctgatcgataccctgcagcgcttggggatcagttaccacttcaaaaacgaaatcaacaccattctgaaaaaaaaatacaacgataactacatcaacaataacattattaccaatccgaactacaacaacctctacgcgaccgcgctggagttccgcctgctgcgccagcatggctacaccgtgccgcaagagatcttcaatgccttcaaagacaagcgcggcaagttcaagaccagcctctcggatgatatcatgggcgtgctgtgcctgtacgaagcctcgttctacgcgatgaagcatgagaacatcctcgaggaggcccgtattttctcgaccaaatgcctgaagaagtatatggagaaaatcgaaaacgaagaggaaaagaaaatcctgctcctgaacgacaacaacatcaactctaacttgctgctcatcaaccacgcctttgaactcccgctgcattggcgcatcacccgctcggaagcgcgttggttcatcgacgaaatctacgagaagaagcaggatatgaacagcaccctgttcgagttcgccaaactggacttcaacatcgttcagtcgactcaccaagaggatctgcaacatctgagccgctggtggcgcgattgtaagctgggcggcaagctgaactttgcgcgcgaccgcctgatggaagccttcctgtgggatgtcggcctgaagttcgaaggcgagttctcctacttccggcggacaaacgcgcgcctcttcgtcctgatcaccatcatcgacgacatctacgatgtgtacggtaccctggaggagctggaactgttcacatcggccgtggagcgctgggatgtgaaactgattaacgaactgccggactacatgaaaatgccgttcttcgtgctgcacaacaccatcaacgaaatgggctttgatgtgctggtcgaacagaacttcgtgaatatcgaatacctgaaaaagtcctgggtggacctgtgtaaatgttacctccaggaagcgaaatggtactacagtggttatcaaccgaccctggaagaatatacagagctgggctggctgtccatcggcgcgagcgtcatcctgatgcacgcgtatttctgctttaccaaccctatcaccaaacaggacttgaaatctctgcagctgcagcaccactacccgaacatcatcaaacaggcttgcctgatcacccgtctggcggacgacctgggcacctcgtctgacgagctgaaccggggcgatgtgccgaagagcatccagtgttatatgtacgacaataacgcgaccgaagacgaagcccgcgaacacattaaattcttgatcagcgagacctggaaggacatgaacaagaaagatgaagacgaaagctgtctctccgagaatttcgtagaggtctgtaagaacatggcccgcaccgcgctgtttatctatgagaacggcgatggccacggcagccagaacagcctgagcaaggaacgcatcagcaccctgattatcaccccgatcaacatcccgaaatga

SEQ ID NO: 52 - DNA, 1626 bp, synthetic construct
  source 1..1626: mol_type = other DNA
atgcgccgcagcgcgaattaccagccatcgctctggcagcacgaatacttactgtcgctcggcaacacctacgtaaaagaggataacgtggaacgcgtgaccctcctgaagcaggaagtgtccaagatgctgaacgaaactgagggtctgctggaacagctggaactgatcgataccctgcagcgcttgggggtgtcgtaccactttgaacaagaaatcaaaaaaaccttgacgaacgtccatgtcaagaatgtccgcgcccataaaaatcgcattgaccgcaaccgctggggcgatctgtacgcgaccgccctggaattccgcctgctccggcaacatggcttctcaatcgcgcaggacgtgttcgatggcaacattggcgtcgatctggacgataaagacatcaaaggcattctctccctctacgaagcgtcttatctgtcgacccgtatcgatacgaagctcaaagaaagcatctattataccactaagcgtctgcgtaaattcgtggaagtgaacaaaaacgagaccaaaagctataccctgcgtcgcatggtgatccacgccctggagatgccgtaccatcgccgggtcggccgcctggaggctcgctggtatatcgaagtgtatggcgagcgccatgatatgaatccgatcctgctggagctcgcgaaactggactttaacttcgtccaagccatccaccaggatgagctgaaaagcttgtcgtcgtggtggagcaaaaccgggctgaccaaacatctggattttgtgcgcgaccgcatcaccgagggctatttctcctcggttggcgtgatgtacgaaccagagtttgcctaccatcgccagatgctgaccaaagttttcatgctgattaccaccatcgacgatatctacgatatctacggcaccctggaggaactgcaactcttcaccacgatcgtagagaaatgggacgtgaaccgcctcgaggagctgccgaactacatgaagctgtgcttcctttgcctggtgaacgagatcaatcagatcggctactttgttctgcgcgataaaggcttcaacgtgatcccctatctgaaggaatcgtgggccgacatgtgtaccaccttcctgaaggaagccaaatggtataagtcgggctacaaacccaacttcgaagagtatatgcagaacggctggatctcgtcgtcggtgccgaccattctgctgcacctgttctgcctgctgtcggaccagaccctggacatcctgggctcctataaccattccgtagtgcgcagcagcgctaccatcctgcggctggctaacgatctggcgaccagcagcgaggagctggcgcgcggtgacactatgaagtcggtccagtgccacatgcacgagaccggggcctctgaagcggagtcgcgcgcctacatccagggcatcatcggggtcgcctgggacgacctgaatatggaaaagaaatcttgccgcctgcaccagggctttctggaagccgcggccaatctgggccgcgtggcgcagtgcgtctaccagtacggcgatggccatggctgtccggataaagccaagaccgtgaaccatgtaagaagcctgctggtgcatcccctgcccctgaattga

SEQ ID NO: 53 - DNA, 1746 bp, synthetic construct
  source 1..1746: mol_type = other DNA
atgccgctgaactcgctgcataacctggaacgcaaacccagcaaggcgtggtcgacgagctgtaccgcccccgccgcccgtctgcaggccagctttagtctgcagcaggaagaaccgcgccagatccgccgttccggcgattaccagccgagcctgtgggatttcaactatatccagtccctgaacaccccgtataaagaacagcgctacgtgaaccggcaggccgaactcattatgcaggtccgcatgctgctcaaggtgaagatggaggcgattcagcagctggaactgatcgatgacctgcaatacctgggcctgagctacttcttcccggatgaaatcaagcaaatcctgtcgagcatccacaatgagcatcgctacttccacaacaacgacctttatcttacggcgctgggcttccgcatcctgcgccaacatggcttcaacgtctcggaagatgtgtttgattgtttcaaaacggaaaaatgtagcgactttaacgcgaacctggcgcaggacaccaagggcatgctgcagctgtacgaagcgagctttctgctgcgcgaaggcgaggacaccctggagctcgcgcgccgcttcagcactcgtagcctgcgtgagaagctggacgaagacggcgatgaaatcgacgaggatctctcgagctggatccgccattcgctggacctgccgctgcattggcgcatccaggggctcgaagcgcgctggtttctggacgcctatgcgcgccgtccggatatgaacccgctgatcttcaaactggcgaaactcaactttaacatcgtgcaggccacctatcaggaagagctgaaggatgtctcgcggtggtggaacagctcctgtctggcggaaaaactgccatttgtgcgcgatcgcatcgtggagtgtttcttctgggcaatcggcgcgttcgagccgcatcagtattcgtaccaacgcaaaatggccgcgatcatcatcaccttcgtgacgattatcgatgatgtgtacgacgtgtatggcaccctggaggaactcgagctgttcaccgacatgatccgccgctgggataacatctcgatctcccaactgccgtattatatgcaggtttgttacctcgcgctttacaacttcgtctcggaacgcgcctatgatatcctgaaagatcagcacttcaactccatcccgtatctgcagcgcagctgggtgagcttggtggaaggctatctgaaagaagcctactggtactacaacggctataaaccctcgctggaggaatatctgaacaatgcgaaaatcagcatcagtgcgccgaccatcatcagccagctgtattttaccctggcgaacagcaccgatgagacggtgatcgagtccctgtacgaataccacaacatcctgtatctgtctggcacgatcctgcgcctggccgatgacctcggcacgtcgcagcatgagctggagcgtggcgatgtgccgaaagcgatccagtgttatatgaaggataccaatgcgagcgaacgcgaagccgttgagcacgtcaaattcctgatccgcgaaacctggaaggaaatgaacaccgtgaccaccgccagcgattgcccgttcacggacgatctggtggcggttgcgacgaacctggcgcgcgccgcgcagttcatctacctcgatggcgacggccatggcgttcagcattcggagatccatcagcagatgggcggcctgctgttccagccgtatgtgtga

SEQ ID NO: 54 - DNA, 1710 bp, synthetic construct
  source 1..1710: mol_type = other DNA
atgtcgaacttcctggtcagcacgtgcagctccccactggccttggacgagaacaactcgacgaaacaagaccatgtcatccgcaatacggtgaccttccatagcagcatctggggcgaccagttcctgacctacgatgagaaagacgacctcgtggcggagaaacagctggccgaggaactgatcgaagaaacccgtaaagaattgatcatcaccaccagctcgcatgagcccatccagcacatgaaactgatccaacttattgatgccgtgcagcggctgggcgtcgcgtatcatttcgagaaagagatcgaagatgccctgcagcatgtctatcgcacgtacggccaccaaggcatccacaacaacaacgacctgcagtctatctcgctctggttccgtatcctccgccagcagggcttcaacgtgagctcggagatcttcaagaaccatatggacgagaaaggcaacctgttcagcaacgatgtgcagtcgatgctcgcgctgtacgaggcctcctatatgcgcgtggaaggcgagaaggtgctcgatgacgccttggaattcacgaagactcacctggccatcatcgctaaagacccgtcgtgcgactcgtcgctgcgcacccaaatccaagatgcgctgcgccagccgctgcgcaagcggctgccgcggctggaggcggtgcgctatatccccatctaccagcagcagtccagccacaatcaaatcctgctcaaactggccaaactcgacttcaacatgctgcagaccatgcacaaaaaagagctgtccgaaatctgtaaatggtggaaagacctggatatgcagaacaagctgccgttcgtgcgcgaccgcctgatagagggctatttctggatcctgggcatctatttcgagccacatcatagccggtctcgtatgttcctgatcaaaagctgcatgtggttggtggtgatcgacgacaccttcgataactacggcacctatgaagagctggaaatcttcaccgaagccgtggaacgctggagcattaactgcctggatatgctgccggagtacatgaaactgatctacaaagagctggtgatcgttcatcaggaaatggaagaaaccctggagaaagaaggtaaagcgtaccatatccaccacgtgaaggaactggccaaagagtgtacccgctccctgctggtggaagcgaaatggctgaaggagggctacatgccgaccctggatgagtatatcagcaacagcctcatcacctgtgcctacgccgttatgatcgcccggagctacgtcggcggcgatgacaaacttgtgaacgaagactcctttaaatgggttgcgacccacccgccgctggtcaaagccagctgtctgatcctgcgtctgatggacgacatcgccacccacaaggaggagcaggaacggggccatgtggcctcgtcgatcgagtgttacatcaaagaaacgggcgccaccgaagaagaggcccgcgagcatttcagcaaacaagtggaagacgcctggaaggtcgtgaatcgcgagtcgctccgcccaacggccgtagccttccccctggtcatgccggcgatcaacctggcccgcatgtgtgacgccctgtacaaaggcaaccacgacgggtataatcacgcgggcaaagaagtcatccagtacatcaaatcgctgctggtccatccgttgatctga

SEQ ID NO: 55 - DNA, 765 bp, synthetic construct
  source 1..765: mol_type = other DNA
atgcgctcgatcgctcgtcgtaccgccgttggtgccgctctgctgttagttatgccggtcgctgtctggatctctgggtggcgttggcaaccgggcgaacagagctggttattaaaagcggcgttctgggtgaccgaaaccgtcacgcagccgtggggtgttattactcatctgatcctatttggctggttcctgtggtgcctacgctttcggatcaaagcggctttcgtgctgtttgcaatattagctgcggccattctggtgggccagggtgtaaaatcgtggattaaagataaagtacaggaaccgcgcccgttcgtgatttggctggaaaaaacacatcatattccggtggatgaattctatactctgaaacgagctgaacgcgggaacctggtcaaagaacaacttgcagaagaaaaaaacattccgcagtatctgcgctctcattggcaaaaggaaaccgggtttgcatttccgagcggacataccatgtttgcggcgagctgggcattactggcggtcggcctactgtggccccgacggagaacactgaccattgcgatcctgttagtgtgggcgaccggagtgatgggatctcgcctgctgctgggcatgcattggccgcgcgacctggtggtggcgaccctaataagttgggcgctggtggcggtggcgacatggctggcgcaacgaatttgcgggccactcacgccgccggctgaagaaaaccgagaaattgcgcaacgtgagcaggaaagttga

SEQ ID NO: 56 - DNA, 1743 bp, synthetic construct
  source 1..1743: mol_type = other DNA
atggcccagatctctatcggcgccccgctgtccgccgaagtcaacggcgcgtgtatcaacacccaccaccatggcaatttatgggatgactatttcatccagtcgctgaaatcgccgtacgaggccccggaatgccacgaacgctgcgaaaagatgatcgaggaagtgaaacatctgctgctgagcgaaatgcgcgacggcaacgatgacctgattaaacgccttcagatggtggatatctttgaatgcctgggcattgatcggcacttccaccatgagattcaggcggccctcgactacgtttaccgctattggaacgaattggaggggatcggcgtgggcacacgcgatagtctgaccaaagacctgtacgcgacaggcctcggtttccgcgccctgcggctgcatcgttataacgtgtccagcgcggtgctggaaaacttcaaaaacgagaacggcctgttcttccactcctccgcggtgcaggaagaagaggtgcgctgtatgctcaccctgctgcgcgcgagcgaaatcagctttccgggggagaaagtcatggacgaagccaaagccttcgcgaccgagtacctgaaccagctgctgacccgcgtggatatcaccgaagtgggcgagaacttgctgcgcgaagtgcgctacgcgctggatttcccatggtattgtagcgtgccgcgctgggaggcgcgcagcttcatcgaaatcttcggccagaataactcctggctgaagtcgacgatgaacaaaaaggtgctggaactggcgaagctggactttaacatcctgcagagcgcccatcagcgggagctgcaactcctgagccgctggtggagccaaagcgatatcgaaaaacagaacttctatcggaaacggcatgtggagttctatttctggatggtgattggcaccttcgagccggaattctcctcatcccgcatcgcgtttgccaaaatcgccaccctgatgacgatcctggacgacctgtacgacacccacggcaccctggagcagctgaagatctttacggaagcggttaagcgctgggacctgtcgctgcaggatcgcctgccggattacatcaaaatcaccctggagttcttcttcaacaccagcaacgaactcaacgcggaagtggcaaaaatgcaagagcgcgatatgtccgcctacattcgcaaagcgggctgggaacgctatattgaaggctacatgcaagagagcgagtggatggccgcccgccatgtgccgaccttcgatgactacatgaagaatggcaaacggagcagcggcatgtgcatcctgaacctgtactcgctgctgctgatgggtcaactggtgccggacaacatcctggagcagatccacctgccgagcaaaatccatgagctggtggaactgaccgcgcggctcgtcgacgactccaaggactttcaagcgaaaaaagatggcggggagttcgcgagcggcacggagtgttatctgaaagaaaaaccggaatgcactgaggaagacgccatgaaccacctgatcggcttgctgaacctgacggccatggagctgaattgggaattcgtcaagcatgacggcgtcgcgctctgtctgaaaaaattcgtgttcgaggtagcccgcggcctgcgcttcatttataagtaccgtgacgggtttgactacagcaacgaggagatgaaaagccagatcaccaaaatcctgattgatcaggtgccgatctga

SEQ ID NO: 57 - DNA, 1770 bp, synthetic construct
  source 1..1770: mol_type = other DNA
atggcggagatgtttaatggcaacagctcgaacgacgggagcagctgcatgccggtaaaagatgccctgcgccgcaccggcaaccatcacccgaacctgtggaccgatgactttatccagagcctgaacagcccgtacagcgattcatcgtatcacaaacatcgcgaaatcctgatcgacgagatccgggacatgttcagcaatggcgaaggcgatgaattcggcgttttggagaacatttggttcgtcgatgtggttcaacgcctcggcatcgaccgccatttccaggaggagattaaaaccgcgctcgactacatctacaaattctggaaccacgattcaatcttcggcgatctgaacatggtggcgctgggctttcgcatcttacgcctgaatcgctacgtcgcttcttctgatgtgttcaaaaaattcaagggggaagagggccagttctcgggcttcgagagcagcgaccaggacgccaagctggagatgatgctgaacctgtacaaagccagcgaactggatttcccggatgaagatatccttaaagaagcccgcgccttcgccagcatgtacctgaagcacgtgatcaaagaatacggcgatatccaagaatcgaagaacccactgctgatggagatcgagtacacctttaaatacccttggcgctgccgcctgccgcggctggaagcatggaactttatccacatcatgcgccagcaggactgcaacatctcgctggccaataacctgtataaaatcccgaaaatctacatgaagaagatcctggagctggcgatcctggacttcaacatcctccagtcgcagcatcagcacgaaatgaaactgattagcacctggtggaagaactcgagcgcgatccaactcgatttcttccgccaccgccatatcgaatcgtatttctggtgggcaagccccctgttcgagccggagttctcaacctgtcgcatcaactgtacgaagctgagcacgaagatgttcctgctcgatgacatttacgacacctatggcactgttgaagagctgaaaccgttcaccacgaccctgacccgctgggatgtctcgacggtagacaaccaccccgactatatgaaaatcgcgtttaacttcagctacgagatctataaagagatcgcgtcggaggcggaacgcaaacatggcccgttcgtgtataagtacctgcaatcgtgttggaaatcgtacatcgaagcgtatatgcaggaagccgaatggatcgcgtccaaccacatcccggggttcgacgagtacctgatgaacggcgtcaaatcctcgggcatgcgcatcctgatgatccacgcgctgatcctcatggataccccgctctcggacgaaatcttggagcaactggacatcccgagctcgaagagccaggccctgctgtcgctcatcacgaggctggtggacgatgtgaaagatttcgaagacgagcaggcgcacggcgaaatggcgagctcaatcgaatgttacatgaaagacaaccatggctccacgcgggaagatgcgttgaactacctgaaaatccgcatcgaatcgtgcgtgcaagagttgaacaaagaactgcttgaaccgtctaatatgcatggctcgtttcgcaacctgtatctgaacgtggggatgcgcgtgatcttcttcatgctgaacgacggtgacctgttcacccacagcaaccgtaaagaaattcaggacgcgatcaccaaattcttcgtggagccgatcatcccgtga

SEQ ID NO: 58 - DNA, 1674 bp, synthetic construct
  source 1..1674: mol_type = other DNA
atggccgcagtggataccaacgcgacgatccaggagaagaccactgcggaaccggtccgtcccctggccaactttcctccgagcgtctggggcgatcggttcctgtcttttaccctggacaattcggagctggagggctacgcgaaagccatggaagccccaaaagaggaactccgccggctgatcgtggatcagacgatggatagcaacaagaagctctcgctgatctattcggtccatcgcctggggctgacctacctgttcctgcaagaaatcgaagcccagctcgacaagatcttcaaagaattcaacttgcagaattacgacgaggtggatctctacacgaccagtatcaacttccaagtcttccgccacctgggctataagctgccgtgcgatgtgttcaacaaattcaaggataatacatccggcgcgttcaaagaggatatctcgaccgacgtcaaaggcatgctgggcctctatgagagctcgcagctgcgcacccgcggcgaaagcatcctcgatgaggcgagctcgtttaccgaaaccaaattgaagtccgtagtgaataatctggagggcaacctggcccagcaggtgctgcagtcgctgcgccgtccgtttcatcaaggcatgccgatggtcgaggcacgtctttacttctcgaattacagcgaagaatgtgcgacccacgagtgtctgctgaaactggcgaagctgcatttcagctatctggaactgcagcagaaggaagaactgcgtatcgtgtcaaaatggtggaaagacatgaggtttcaggagacgacgccttatatccgcgatcgggtgccggagatttatttatggattctgggcctgtatttcgagccgcggtattctctggcccggatcatcgcgacgaagatcaccctctttctggtggtgctggatgatacgtatgatgcgtatgcgaccatcgaagagatccgtctcctgaccgatgcgatcaaccgctgggacatgagcgcgatggagcagatccccgaatatatccgcccgttctataaaatcctcttggatgaatatgccgaattggagaagcaactggccatcgaaggcagagcaaaaagcgtcatcgcgagcaaagaggccttccaggatatcgctcgcggctatcttgaggaagcggaatggacgaacagtggctacgtcgcgtctttcccggagtatatgaagaacggcctgatcactagcgcctataacgtcatcagcaaatccgccctggtcggcatgggcgatattgtgtccgaaaacgcgctggcctggtacgagagccaccctaaaaccctgcaggcctcggaactgatttcgcgcctgcaggacgacgtcatgacgtaccagttcgaacgcgagcgcggccagtctgcgacgggcgtggatgcgtacatcaaaacgtacggcgtctcggagaaagaggccatcgacgaactgaagatcatgatcgagaacgcttggaaagatatcaacgagggctgcctcaagccgcgagaggtgtctatggatctcttggcccctatcctgaacctcgcgcgcatgatcgacgtcgtctatcgttacgacgacggcttcaccttcccgggcaagaccatgaaggagtacatcaccttactgttcgtcggctccgtgtcgatgtga

SEQ ID NO: 59 - DNA, 1725 bp, synthetic construct
  source 1..1725: mol_type = other DNA
atgtcgactctgcccatctcgtcggtgagcttttcaagcagcacgagcccgctggtcgttgatgacaaagtctcgaccaaacccgatgtcatccgccacaccatgaacttcaacgccagcatctggggcgaccagttcctgacctacgacgagcctgaagacctggtgatgaaaaaacagctggtcgaagaactgaaagaggaagtgaagaaggaactgatcacgattaagggcagcaacgaaccaatgcagcatgtgaagctgatcgaactgatcgacgctgtgcagcgcctgggcatcgcctatcatttcgaagaagaaattgaggaggcgctgcagcacatccacgtaacctatggcgagcagtgggtggataaagagaacctgcagagcatatccctgtggttccgcctgctgcgccagcagggcttcaacgtgtcgagcggcgtgttcaaggacttcatggacgaaaagggtaagttcaaagaatcgctgtgtaacgatgcccagggcatcctggcgctgtatgaggccgcgttcatgcgcgtggaggacgagacgatactggataacgcgctggagttcaccaaggtgcatctggacatcatcgccaaagacccctcgtgtgatagctcgctgcgcacccagatccaccaggccctgaaacagcctctgcgccgccgtctggcccgcatcgaagccctgcattacatgccgatctatcagcaggaaacctcgcatgacgaagtgctgctgaaactggcgaaactggacttttcagtgctgcagagcatgcataagaaggaactgtcgcatatttgcaagtggtggaaagatctggatctgcagaacaaactgccgtacgtcagggaccgcgtggtggaaggctacttttggatactctcgatctactatgagccacagcatgcccgcacccgcatgttcctgatgaagacctgtatgtggctcgtcgttctggatgacaccttcgacaactacggcacctacgaggaactcgaaatctttacccaggcggtggagcgctggagcatctcgtgtttggatatgctgccggagtacatgaagctgatctatcaggaactggtcaacctgcatgtggaaatggaagagtctctggaaaaagaggggaaaacgtaccagatccactacgttaaagagatggcgaaagagcttgtgcggaactatctggtggaggcacgctggctcaaagaaggctacatgccgaccctggaggagtacatgtcggtcagcatggtgacgggcacctatggtctgatgattgcgcgctcctatgtgggccgaggcgatatcgtgaccgaagacaccttcaaatgggtttcgtcctacccgccgattatcaaggcgagctgtgtgatcgtgcgcctgatggacgacatcgtgagccacaaagaagagcaagaacgcggccacgtggcctcttctatcgaatgctatagcaaagagagcggcgcgagcgaggaggaggcgtgtgagtatatcagccgcaaagtggaggatgcctggaaagttatcaaccgcgagtcgctgcgcccgaccgcggtgccgttcccactgctgatgccggcgatcaacctggcgcgcatgtgtgaagtgctgtacagtgtgaacgatggcttcacccacgcggaaggcgatatgaaatcgtatatgaaaagcttcttcgtgcatccgatggtggtgtga

SEQ ID NO: 60 - DNA, 1659 bp, synthetic construct
  source 1..1659: mol_type = other DNA
atggaactgtatgcccaaagcgtcggcgtgggcgcggcgtcccggcccctggcgaactttcatcagtgcgtgtggggcggcaaatttattgtgtataacccgcagtcctcgcaagccggcgaacgcgaacaggcggaggagctcaaggtggagctgaaacgcgaactgaaggaggcgagcgataactacatgcgccagctgaaaatggtggacgccattcagcggctgggcatcgattacctgtttgtggaagatgtcgacgaggcgctgaaaaatctgttcgaaatgtttgacgccttctgcaaaaacaaccatgatatgcatgcgaccgccctgtcgttccgcctgctgcgccagcatggctaccgcgtgagctgtgaagtgttcgaaaaattcaaagatggcaaagacggcttcaaagtcccgaacgaagacggcgcggtcgcggtgctggagttttttgaggccacccacctgcgggtgcacggcgaggatgtgctggacaacgcgttcgtgttcacccgcaactatctggaatccgtgtatgcgaccctgaacgacccgaccgcgaatcaggtgcataacgcactgaacgaattcagcttccgccgcggcctgccgcgcgttgaagcgcgcaaatacattagcatctatgaacagtatgcgagccatcacaaaggcctgctgcggctggccaaactggactttaacctggtgcaggcgctgcatcgccgcgagctgtcggaggactcgcgctggtggaaaaccctgcaggtgccgaccgaactgagctttgtgcgcgaccggctggtggagagctatttctgggcgtccggcagctacttcgaaccgaactacagcgtggcgcggatgatcctggccaagggcctggcggtcctgagcctgatggacgacgtgtatgatgcgtatggcacctttgaagaactgcaggtcttcaccgacgcgatcgaacgctgggatgcactgtgtttggacaaactgccagagtacatgaaaattgtgtataaagcgctcctggatgtgttcgaagaggtggatgaagaagtcattaagctgggcgccccgtaccgtgtctattatggcaaggaagcgatgaaatacgccgcgcgggcctacatggaagaggcgcagtggcgcgagcagaaacataaaccgaccaccaaagaatatatgaagctggcgaccaagacctgtggctatatcacgctgatcatcctgagcttcctgggcgtggaagaaggcatcgtgacgaaagaagcgttcgactgggttttctcgcgcccgccgttcgtggaggccaccctgatcattgcccgcctgatcaacgatatcaccggctgcgaattcgaaaacaaacgcgagcatgtgcgcaccgccgtcgagtgctacatggaagaacataaggtaggcaaacaggaagtcgtgagcgaattttacaaccagatggaatctgcctggaaagacattaacgaatgtctgctgcgcccggcggaatttccgatccctctgctgaacctgattctgaactcggtgcgcacgctggaagtaatctataaagaaggcgatagctatacccacgtgggcccggccatgcagaacattattaaacagctgtacctgcatccggtcccgtactga

SEQ ID NO: 61 - DNA, 1014 bp, synthetic construct
  source 1..1014: mol_type = other DNA
atgccgcaggatgttgattttcatatccctctgcccggccgacaatctccagaccatgcgagagcagaagctgaacaacttgcttggccgcgctcgctgggactcattcggagcgacgcggcggcggaacgccatttgcgcggaggttatgctgatttggcaagccgcttttatccacacgctacgggggccgatctggacctgggcgtggacctgatgagctggtttttcctcttcgacgatctttttgatggcccgcgcggcgaaaatccggaagatacgaaacaactaactgaccaggttgcagctgcgctagatggcccgctgcccgacacggcacctccgattgctcatggctttgcagatatctggcgtcggacgtgcgaaggcatgactccggcttggtgcgcgcgatctgcccgccattggcgaaattatttcgatggttatgtggatgaagcggaaagccgattctggaacgcgccgtgcgatagcgccgcgcagtatctggccatgcgccgccacacgattggagtacaaccgacagtggatctggcagaacgagcgggccgctttgaagtgcctcaccgggtctttgactctgcggtcatgagtgctatgttgcagattgctgtcgatgtcaatctgctgttaaacgacatcgcgtcactcgaaaaagaagaagctcgaggcgaacaaaacaatatggtaatgattcttaggcgcgaacacggctggtctaaatcccgctcggttagtcatatgcaaaatgaagtgcgcgcccgactggagcaatacctcctgctcgaatcctgtctccctaaagtcggcgaaatctatcaactggataccgcagaacgcgaagcgttagaaagatatcgcaccgatgcggtccggaccgttattcgtggtagttacgattggcaccgcagctcgggcaggtatgatgcggaattcgcacttgcggctggcgcccaaggctaccttgaagaactgggaagcagcgcgcactga

SEQ ID NO: 62 - DNA, 1656 bp, synthetic construct
  source 1..1656: mol_type = other DNA
atgagcacccaacaggtgtcctctgagaacatcgtgcggaacgcggcgaacttccatccaaacatctgggggaaccacttcctgacctgcccctcccagaccattgattcgtggacccagcagcaccacaaggagctgaaagaggaggtgcgcaaaatgatggtgtcggatgccaacaaaccggcgcagcgcctgcgcctgatcgacacggtccagcggctgggcgtggcctaccatttcgaaaaggagatcgacgatgccctggaaaaaatcggccacgacccgttcgatgacaaagatgacctgtatatcgtgtcgctgtgttttcgcctgctgcgccagcacggaattaagatcagctgcgacgtcttcgaaaaattcaaggatgatgatggcaaattcaaggcgagcctgatgaacgacgtgcagggcatgctgtcgttgtacgaagccgcgcatcttgccatccacggcgaggacatcctggacgaggccatcgtcttcaccacgacccacttaaagagcaccgttagcaacagcccggtgaacagcaccttcgccgaacaaattcgccattccctgcgagtgccgctgcgcaaggccgttccgcgcctggaaagccgctattttctggacatctacagccgtgacgatctccatgacaagaccttgctgaactttgcgaagctggacttcaacatcctgcaggccatgcaccagaaggaagccagcgagatgacgcgctggtggcgcgatttcgacttcctgaaaaaactgccgtacattcgggatcgcgttgtcgaactgtatttttggatcctggtcggcgtgagctatcagccgaaattcagcactggtcgcatctttctgagcaaaatcatctgtttggagacgctggttgatgatacgttcgacgcctacggcacctttgatgagctggccatctttacggaagccgtgacgcgctgggacctgggccatcgcgacgcgctgccggaatatatgaagttcatttttaaaaccctgatcgacgtgtactccgaggcggaacaggagctggccaaagaaggccgcagctacagcatccattacgcgatccgctccttccaggaacttgtgatgaaatacttttgtgaggccaaatggctgaacaaaggctacgtgccgtcgctcgacgactacaagagcgtgtctctgcgctctattgggttcctcccgatcgcggtggcgagcttcgtgttcatgggcgacatcgccaccaaagaagtgttcgagtgggagatgaacaatccgaaaatcatcatcgcggcggaaaccattttccgattcctggacgatatcgcgggtcacagatttgaacagaaacgcgagcatagcccgtcggcgatcgaatgttacaaaaaccagcatggcgtgtcggaagaggaagccgtgaaagcgctgtccttggaggtggcaaattcttggaaggacatcaacgaggagctgctgctcaacccgatggcgatccccctgccgctgctgcaggtgattctggacctgtcgcgttcggcagatttcatgtatggcaacgcccaggatcgcttcacccactccaccatgatgaaagatcaggtcgatttggtgctgaaagacccggtgaaactggacgattga

SEQ ID NO: 63 - DNA, 1332 bp, synthetic construct
  source 1..1332: mol_type = other DNA
atgtcattaccgttcttaacttctgcaccgggaaaggttattatttttggtgaacactctgctgtgtacaacaagcctgccgtcgctgctagtgtgtctgcgttgagaacctacctgctaataagcgagtcatctgcaccagatactattgaattggacttcccggacattagctttaatcataagtggtccatcaatgatttcaatgccatcaccgaggatcaagtaaactcccaaaaattggccaaggctcaacaagccaccgatggcttgtctcaggaactcgttagtcttttggatccgttgttagctcaactatccgaatccttccactaccatgcagcgttttgtttcctgtatatgtttgtttgcctatgcccccatgccaagaatattaagttttctttaaagtctactttacccatcggtgctgggttgggctcaagcgcctctatttctgtatcactggccttagctatggcctacttgggggggttaataggatctaatgacttggaaaagctgtcagaaaacgataagcatatagtgaatcaatgggccttcataggtgaaaagtgtattcacggtaccccttcaggaatagataacgctgtggccacttatggtaatgccctgctatttgaaaaagactcacataatggaacaataaacacaaacaattttaagttcttagatgatttcccagccattccaatgatcctaacctatactagaattccaaggtctacaaaagatcttgttgctcgcgttcgtgtgttggtcaccgagaaatttcctgaagttatgaagccaattctagatgccatgggtgaatgtgccctacaaggcttagagatcatgactaagttaagtaaatgtaaaggcaccgatgacgaggctgtagaaactaataatgaactgtatgaacaactattggaattgataagaataaatcatggactgcttgtctcaatcggtgtttctcatcctggattagaacttattaaaaatctgagcgatgatttgagaattggctccacaaaacttaccggtgctggtggcggcggttgctctttgactttgttacgaagagacattactcaagagcaaattgacagcttcaaaaagaaattgcaagatgattttagttacgagacatttgaaacagacttgggtgggactggctgctgtttgttaagcgcaaaaaatttgaataaagatcttaaaatcaaatccctagtattccaattatttgaaaataaaactaccacaaagcaacaaattgacgatctattattgccaggaaacacgaatttaccatggacttcataa

SEQ ID NO: 64 - DNA, 1356 bp, synthetic construct
  source 1..1356: mol_type = other DNA
atgtcagagttgagagccttcagtgccccagggaaagcgttactagctggtggatatttagttttagatacaaaatatgaagcatttgtagtcggattatcggcaagaatgcatgctgtagcccatccttacggttcattgcaagggtctgataagtttgaagtgcgtgtgaaaagtaaacaatttaaagatggggagtggctgtaccatataagtcctaaaagtggcttcattcctgtttcgataggcggatctaagaaccctttcattgaaaaagttatcgctaacgtatttagctactttaaacctaacatggacgactactgcaatagaaacttgttcgttattgatattttctctgatgatgcctaccattctcaggaggatagcgttaccgaacatcgtggcaacagaagattgagttttcattcgcacagaattgaagaagttcccaaaacagggctgggctcctcggcaggtttagtcacagttttaactacagctttggcctccttttttgtatcggacctggaaaataatgtagacaaatatagagaagttattcataatttagcacaagttgctcattgtcaagctcagggtaaaattggaagcgggtttgatgtagcggcggcagcatatggatctatcagatatagaagattcccacccgcattaatctctaatttgccagatattggaagtgctacttacggcagtaaactggcgcatttggttgatgaagaagactggaatattacgattaaaagtaaccatttaccttcgggattaactttatggatgggcgatattaagaatggttcagaaacagtaaaactggtccagaaggtaaaaaattggtatgattcgcatatgccagaaagcttgaaaatatatacagaactcgatcatgcaaattctagatttatggatggactatctaaactagatcgcttacacgagactcatgacgattacagcgatcagatatttgagtctcttgagaggaatgactgtacctgtcaaaagtatcctgaaatcacagaagttagagatgcagttgccacaattagacgttcctttagaaaaataactaaagaatctggtgccgatatcgaacctcccgtacaaactagcttattggatgattgccagaccttaaaaggagttcttacttgcttaatacctggtgctggtggttatgacgccattgcagtgattactaagcaagatgttgatcttagggctcaaaccgctaatgacaaaagattttctaaggttcaatggctggatgtaactcaggctgactggggtgttaggaaagaaaaagatccggaaacttatcttgataaataa

SEQ ID NO: 65 - DNA, 1191 bp, synthetic construct
  source 1..1191: mol_type = other DNA
atgaccgtttacacagcatccgttaccgcacccgtcaacatcgcaacccttaagtattgggggaaaagggacacgaagttgaatctgcccaccaattcgtccatatcagtgactttatcgcaagatgacctcagaacgttgacctctgcggctactgcacctgagtttgaacgcgacactttgtggttaaatggagaaccacacagcatcgacaatgaaagaactcaaaattgtctgcgcgacctacgccaattaagaaaggaaatggaatcgaaggacgcctcattgcccacattatctcaatggaaactccacattgtctccgaaaataactttcctacagcagctggtttagcttcctccgctgctggctttgctgcattggtctctgcaattgctaagttataccaattaccacagtcaacttcagaaatatctagaatagcaagaaaggggtctggttcagcttgtagatcgttgtttggcggatacgtggcctgggaaatgggaaaagctgaagatggtcatgattccatggcagtacaaatcgcagacagctctgactggcctcagatgaaagcttgtgtcctagttgtcagcgatattaaaaaggatgtgagttccactcagggtatgcaattgaccgtggcaacctccgaactatttaaagaaagaattgaacatgtcgtaccaaagagatttgaagtcatgcgtaaagccattgttgaaaaagatttcgccacctttgcaaaggaaacaatgatggattccaactctttccatgccacatgtttggactctttccctccaatattctacatgaatgacacttccaagcgtatcatcagttggtgccacaccattaatcagttttacggagaaacaatcgttgcatacacgtttgatgcaggtccaaatgctgtgttgtactacttagctgaaaatgagtcgaaactctttgcatttatctataaattgtttggctctgttcctggatgggacaagaaatttactactgagcagcttgaggctttcaaccatcaatttgaatcatctaactttactgcacgtgaattggatcttgagttgcaaaaggatgttgccagagtgattttaactcaagtcggttcaggcccacaagaaacaaacgaatctttgattgacgcaaagactggtctaccaaaggaataa

SEQ ID NO: 66 - DNA, 549 bp, synthetic construct
  source 1..549: mol_type = other DNA
atgcaaacggaacacgtcattttattgaatgcacagggagttcccacgggtacgctggaaaagtatgccgcacacacggcagacacccgcttacatctcgcgttctccagttggctgtttaatgccaaaggacaattattagttacccgccgcgcactgagcaaaaaagcatggcctggcgtgtggactaactcggtttgtgggcacccacaactgggagaaagcaacgaagacgcagtgatccgccgttgccgttatgagcttggcgtggaaattacgcctcctgaatctatctatcctgactttcgctaccgcgccaccgatccgagtggcattgtggaaaatgaagtgtgtccggtatttgccgcacgcaccactagtgcgttacagatcaatgatgatgaagtgatggattatcaatggtgtgatttagcagatgtattacacggtattgatgccacgccgtgggcgttcagtccgtggatggtgatgcaggcgacaaatcgcgaagccagaaaacgattatctgcatttacccagcttaaataa

SEQ ID NO: 67 - DNA, 900 bp, synthetic construct
  source 1..900: mol_type = other DNA
atggactttccgcagcaactcgaagcctgcgttaagcaggccaaccaggcgctgagccgttttatcgccccactgccctttcagaacactcccgtggtcgaaaccatgcagtatggcgcattattaggtggtaagcgcctgcgacctttcctggtttatgccaccggtcatatgttcggcgttagcacaaacacgctggacgcacccgctgccgccgttgagtgtatccacgcttactcattaattcatgatgatttaccggcaatggatgatgacgatctgcgtcgcggtttgccaacctgccatgtgaagtttggcgaagcaaacgcgattctcgctggcgacgctttacaaacgctggcgttctcgattttaagcgatgccgatatgccggaagtgtcggaccgcgacagaatttcgatgatttctgaactggcgagcgccagtggtattgccggaatgtgcggtggtcaggcattagatttagacgcggaaggcaaacacgtacctctggacgcgcttgagcgtattcatcgtcataaaaccggcgcattgattcgcgccgccgttcgccttggtgcattaagcgccggagataaaggacgtcgtgctctgccggtactcgacaagtatgcagagagcatcggccttgccttccaggttcaggatgacatcctggatgtggtgggagatactgcaacgttgggaaaacgccagggtgccgaccagcaacttggtaaaagtacctaccctgcacttctgggtcttgagcaagcccggaagaaagcccgggatctgatcgacgatgcccgtcagtcgctgaaacaactggctgaacagtcactcgatacctcggcactggaagcgctagcggactacatcatccagcgtaataaataa

SEQ ID NO: 68 - DNA, 1185 bp, synthetic construct
  source 1..1185: mol_type = other DNA
atgaaaaattgtgtcatcgtcagtgcggtacgtactgctatcggtagttttaacggttcactcgcttccaccagcgccatcgacctgggggcgacagtaattaaagccgccattgaacgtgcaaaaatcgattcacaacacgttgatgaagtgattatgggtaacgtgttacaagccgggctggggcaaaatccggcgcgtcaggcactgttaaaaagcgggctggcagaaacggtgtgcggattcacggtcaataaagtatgtggttcgggtcttaaaagtgtggcgcttgccgcccaggccattcaggcaggtcaggcgcagagcattgtggcggggggtatggaaaatatgagtttagccccctacttactcgatgcaaaagcacgctctggttatcgtcttggagacggacaggtttatgacgtaatcctgcgcgatggcctgatgtgcgccacccatggttatcatatggggattaccgccgaaaacgtggctaaagagtacggaattacccgtgaaatgcaggatgaactggcgctacattcacagcgtaaagcggcagccgcaattgagtccggtgcttttacagccgaaatcgtcccggtaaatgttgtcactcgaaagaaaaccttcgtcttcagtcaagacgaattcccgaaagcgaattcaacggctgaagcgttaggtgcattgcgcccggccttcgataaagcaggaacagtcaccgctgggaacgcgtctggtattaacgacggtgctgccgctctggtgattatggaagaatctgcggcgctggcagcaggccttacccccctggctcgcattaaaagttatgccagcggtggcgtgccccccgcattgatgggtatggggccagtacctgccacgcaaaaagcgttacaactggcggggctgcaactggcggatattgatctcattgaggctaatgaagcatttgctgcacagttccttgccgttgggaaaaacctgggctttgattctgagaaagtgaatgtcaacggcggggccatcgcgctcgggcatcctatcggtgccagtggtgctcgtattctggtcacactattacatgccatgcaggcacgcgataaaacgctggggctggcaacactgtgcattggcggcggtcagggaattgcgatggtgattgaacggttgaattaa

SEQ ID NO: 69 - DNA, 1476 bp, synthetic construct
  source 1..1476: mol_type = other DNA
atgaaactctcaactaaactttgttggtgtggtattaaaggaagacttaggccgcaaaagcaacaacaattacacaatacaaacttgcaaatgactgaactaaaaaaacaaaagaccgctgaacaaaaaaccagacctcaaaatgtcggtattaaaggtatccaaatttacatcccaactcaatgtgtcaaccaatctgagctagagaaatttgatggcgtttctcaaggtaaatacacaattggtctgggccaaaccaacatgtcttttgtcaatgacagagaagatatctactcgatgtccctaactgttttgtctaagttgatcaagagttacaacatcgacaccaacaaaattggtagattagaagtcggtactgaaactctgattgacaagtccaagtctgtcaagtctgtcttgatgcaattgtttggtgaaaacactgacgtcgaaggtattgacacgcttaatgcctgttacggtggtaccaacgcgttgttcaactctttgaactggattgaatctaacgcatgggatggtagagacgccattgtagtttgcggtgatattgccatctacgataagggtgccgcaagaccaaccggtggtgccggtactgttgctatgtggatcggtcctgatgctccaattgtatttgactctgtaagagcttcttacatggaacacgcctacgatttttacaagccagatttcaccagcgaatatccttacgtcgatggtcatttttcattaacttgttacgtcaaggctcttgatcaagtttacaagagttattccaagaaggctatttctaaagggttggttagcgatcccgctggttcggatgctttgaacgttttgaaatatttcgactacaacgttttccatgttccaacctgtaaattggtcacaaaatcatacggtagattactatataacgatttcagagccaatcctcaattgttcccagaagttgacgccgaattagctactcgcgattatgacgaatctttaaccgataagaacattgaaaaaacttttgttaatgttgctaagccattccacaaagagagagttgcccaatctttgattgttccaacaaacacaggtaacatgtacaccgcatctgtttatgccgcctttgcatctctattaaactatgttggatctgacgacttacaaggcaagcgtgttggtttattttcttacggttccggtttagctgcatctctatattcttgcaaaattgttggtgacgtccaacatattatcaaggaattagatattactaacaaattagccaagagaatcaccgaaactccaaaggattacgaagctgccatcgaattgagagaaaatgcccatttgaagaagaacttcaaacctcaaggttccattgagcatttgcaaagtggtgtttactacttgaccaacatcgatgacaaatttagaagatcttacgatgttaaaaaataa

SEQ ID NO: 70 - DNA, 13 bp, synthetic construct
  source 1..13: mol_type = other DNA
aaagaggggacaa

SEQ ID NO: 71 - DNA, 13 bp, synthetic construct
  source 1..13: mol_type = other DNA
aaagataggagac

SEQ ID NO: 72 - DNA, 13 bp, synthetic construct
  source 1..13: mol_type = other DNA
aaagactggagac

SEQ ID NO: 73 - DNA, 13 bp, synthetic construct
  source 1..13: mol_type = other DNA
aaagaggtgacat

SEQ ID NO: 74 - DNA, 13 bp, synthetic construct
  source 1..13: mol_type = other DNA
aaagaagggatac

SEQ ID NO: 75 - DNA, 13 bp, synthetic construct
  source 1..13: mol_type = other DNA
aaagaaacgacat

(19) United States
(12) Patent Application Publication
(10) Pub. No.: US 2025/0290102 A1
(43) Pub. Date: Sep. 18, 2025

(54) Production of terpenoid compound and the strain used by

(71) Applicant: Xi'an Zhuohong New Energy Biotechnology Co., Ltd., Xi'an City (CN)

(72) Inventors: Yujie CAI, Xi'an City (CN); Di LIU, Xi'an City (CN); Linmeng ZHANG, Xi'an City (CN); Long WANG, Xi'an City (CN); Yao LU, Xi'an City (CN); Xia CHEN, Xi'an City (CN); Songsong YAO, Xi'an City (CN); Yongai MA, Xi'an City (CN); Yi YAN, Xi'an City (CN); Xiaoyuan ZHANG, Xi'an City (CN); Yanrui DING, Xi'an City (CN); Linbo GOU, Xi'an City (CN)

(21) Appl. No.: 19/223,198

(22) Filed: May 30, 2025

Related U.S. Application Data
(63) Continuation of application No. PCT/CN2024/110540, filed on Aug. 8, 2024.

(30) Foreign Application Priority Data
Sep. 19, 2023 (CN) 2023112209708

(51) Int. Cl. C12P 5/00; C12N 9/04; C12N 9/10; C12N 9/12; C12N 9/16; C12N 9/88; C12N 9/90

(52) U.S. Cl. CPC C12P 5/007; C12N 9/0006; C12N 9/1025; C12N 9/1029; C12N 9/1085; C12N 9/1205; C12N 9/1229; C12N 9/16; C12N 9/88; C12N 9/90; C12Y 101/01088; C12Y 203/01009; C12Y 203/0301; C12Y 205/01001; C12Y 205/0101; C12Y 207/01036; C12Y 207/04002; C12Y 301/07011; C12Y 401/01033; C12Y 402/03007; C12Y 402/03015; C12Y 402/03016; C12Y 402/0302; C12Y 402/03023; C12Y 402/03026; C12Y 402/03027; C12Y 402/03047; C12Y 402/03058; C12Y 402/0307; C12Y 402/03073; C12Y 402/03082; C12Y 402/03106; C12Y 402/03108; C12Y 402/03114; C12Y 503/03002

(57) ABSTRACT

Disclosed are production of terpenoid compound and the strain used by, which belong to the technical field of bioengineering. The disclosure constructs an engineered strain of Serratia marcescens in production of hemiterpenes or monoterpenes, and the engineered strain of S. marcescens can produce linalool, isoprene, isopentenol, 1,8-cineole, α-pinene, pinene, γ-terpinene, geraniol, (+)-limonene, (−)-limonene, myrcene, β-ocimene, sabinene, (−)-α-bisabolol, farnesol, longifolene, valencene, β-elemene, farnesene, patchoulol, pentalenene, and α-santalene. In a 30 L fermenter, the yield of linalool produced by the engineered strain of S. marcescens is 40.72 g·L⁻¹.

### REFERENCE TO SEQUENCE LISTING

The instant application contains a Sequence listing in XML format as a file named “YGHY-2025-09-SEQ.xml”, created on May 27,2025, of 135,466 bytes in size, and which is hereby incorporated by reference in its entirety.

### TECHNICAL FIELD

The disclosure relates to production of terpenoid compound and the strain used by, and belongs to the technical field of bioengineering.

### BACKGROUND

Terpenoids (terpenes), also known as isoprenoids, are the most diverse class of natural products with over 75,000 known species. Terpenoids are highly demanded on the market and have a wide range of applications in many industries. For example, they may be used as a biofuel (isoprenoid), an antimalarial drug (artemisinin), an anti-cancer drug (paclitaxel), a coloring agent for food and feed (carotenoid), a natural organic solvent in household cleaning agents (limonene), and the like.

Microorganisms produce terpenoid C₅ precursors isopentenyl diphosphate (IPP) and dimethylallyl diphosphate (DMAPP) in vivo through a 2-C-methyl-D-erythritol 4-phosphate pathway (MEP pathway) or a mevalonate pathway (MVA pathway). The C₅ precursors may be directly prepared into hemiterpenes (C₅) through hemiterpene synthase, or condensed to produce longer pyrophosphate intermediates such as geranyl pyrophosphate (GPP, C₁₀), farnesyl diphosphate (FPP, C₁₅), and geranyl geranyl pyrophosphate (GGPP, C₂₀). The pyrophosphate intermediates are converted into terpenoids such as monoterpenes (C₁₀), sesquiterpenes (C₁₅), and diterpenes (C₂₀) under the action of terpene synthase.

In recent years, with the development of metabolic engineering and synthetic biology, the production of terpenoids has been carried out in microorganisms, mainly using Escherichia coli and Saccharomyces cerevisiae as host cells. However, the yield of terpenoids by the above host cells can only reach 1-2 g·L⁻¹, which cannot meet the needs of industrial production. Due to the high market demand for terpenoids, an expression system for producing terpenoids is still in need.

### SUMMARY

The disclosure provides a method for large-scale production of terpenoids using a mevalonate (MVA) pathway in Serratia marcescens. Examples of the terpenoids include: hemiterpenes (C₅) such as isoprene and isopentenol; monoterpenes (C₁₀) such as 1,8-cineole, linalool, α-pinene, β-pinene, γ-terpinene, geraniol, limonene, myrcene, β-ocimene, and sabinene; and sesquiterpenes (C₁₅) such as bisabolol, farnesol, longifolene, valencene, elemene, farnesene, patchoulol, pentalenene, and santalene.

The disclosure provides an engineered strain of S. marcescens, and Serratia marcescens (S. marcescens) HBQA7 is used as a chassis cell to expresses acetyl-CoA acetyltransferase, HMG-CoA synthase, HMG-CoA reductase, mevalonate kinase, phosphomevalonate kinase, and mevalonate pyrophosphate decarboxylase.

The accession number of the S. marcescens HBQA7 is CCTCC NO: M 2023184. The strain S. marcescens HBQA7 has the property of being organic solvent-tolerant.

In an implementation, the engineered strain of S. marcescens that expresses the acetyl-CoA acetyltransferase, HMG-COA synthase, HMG-COA reductase, mevalonate kinase, phosphomevalonate kinase, and mevalonate pyrophosphate decarboxylase also expresses a combination of any of the following enzymes:

(1) isopentenyl pyrophosphate isomerase, geranyl pyrophosphate synthase, and any one of (R)-linalool synthase, 1,8-cineole synthase, α-pinene synthase, pinene synthase, γ-terpinene synthase, geraniol synthase, (+)-limonene synthase, (−)-limonene synthase, myrcene synthase, β-ocimene synthase, or sabinene synthase;

(2) isopentenyl pyrophosphate isomerase, farnesyl pyrophosphate synthase, and any one of (−)-α-bisabolol synthase, phosphatase, longifolene synthase, valencene synthase, germacrene A synthase, farnesene synthase, patchoulol synthase, pentalenene synthase, or α-santalene synthase; and

(3) isoprene synthase or isopentenol synthase.

In an implementation, the amino acid sequence of the acetyl-CoA acetyltransferase is shown as SEQ ID No: 1, the amino acid sequence of the HMG-COA synthase is shown as SEQ ID No: 2, the amino acid sequence of the HMG-COA reductase is shown as SEQ ID No: 3, the amino acid sequence of the mevalonate kinase is shown as SEQ ID No: 4, the amino acid sequence of the phosphomevalonate kinase is shown as SEQ ID No: 5, the amino acid sequence of the mevalonate pyrophosphate decarboxylase is shown as SEQ ID No: 6, the amino acid sequence of the isopentenyl pyrophosphate isomerase is shown as SEQ ID No: 7, the amino acid sequence of the geranyl pyrophosphate synthase is shown as SEQ ID No: 8, the amino acid sequence of the (R)-linalool synthase is shown as SEQ ID No: 9, the amino acid sequence of the 1,8-cineole synthase is shown as SEQ ID No: 10, the amino acid sequence of the α-pinene synthase is shown as SEQ ID No: 11, the amino acid sequence of the pinene synthase is shown as SEQ ID No: 12, the amino acid sequence of the γ-terpinene synthase is shown as SEQ ID No: 13, the amino acid sequence of the geraniol synthase is shown as SEQ ID No: 14, the amino acid sequence of the (+)-limonene synthase is shown as SEQ ID No: 15, the amino acid sequence of the (−)-limonene synthase is shown as SEQ ID No: 16, the amino acid sequence of the myrcene synthase is shown as SEQ ID No: 17, the amino acid sequence of the β-ocimene synthase is shown as SEQ ID No: 18, the amino acid sequence of the sabinene synthase is shown as SEQ ID No: 19, the amino acid sequence of the (−)-α-bisabolol synthase is shown as SEQ ID No: 20, the amino acid sequence of the farnesyl pyrophosphate synthase is shown as SEQ ID No: 21, the amino acid sequence of the phosphatase is shown as SEQ ID No: 22, the amino acid sequence of the longifolene synthase is shown as SEQ ID No: 23, the amino acid sequence of the valencene synthase is shown as SEQ ID No: 24, the amino acid sequence of the germacrene A synthase is shown as SEQ ID No: 25, the amino acid sequence of the farnesene synthase is shown as SEQ ID No: 26, the amino acid sequence of the patchoulol synthase is shown as SEQ ID No: 27, the amino acid sequence of the pentalenene synthase is shown as SEQ ID No: 28, the amino acid sequence of the α-santalene synthase is shown as SEQ ID No: 29, the amino acid sequence of the isoprene synthase is shown as SEQ ID No: 30, and the amino acid sequence of the isopentenol synthase is shown as SEQ ID No: 31.

In an implementation, a nucleotide sequence encoding the acetyl-CoA acetyltransferase is shown as SEQ ID No: 33, a nucleotide sequence encoding the HMG-COA synthase is shown as SEQ ID No: 38, a nucleotide sequence encoding the HMG-COA reductase is shown as SEQ ID No: 39, a GenBank accession number of a nucleotide sequence encoding the mevalonate kinase is NC_001145 REGION: 684467 . . . 685798 (SEQ ID No: 63), a GenBank accession number of a nucleotide sequence encoding the phosphomevalonate kinase is NC_001145 REGION: 712316 . . . 713671 (SEQ ID No: 64), a GenBank accession number of a nucleotide sequence encoding the mevalonate pyrophosphate decarboxylase is NC_001146 REGION: 701895 . . . 703085 (SEQ ID No: 65), a GenBank accession number of a nucleotide sequence encoding the isopentenyl pyrophosphate isomerase is NC_000913 REGION: 3033065 . . . 3033613 (SEQ ID No: 66), a nucleotide sequence encoding the geranyl pyrophosphate synthase is shown as SEQ ID No: 40, a nucleotide sequence encoding the 1,8-cineole synthase is shown as SEQ ID No: 44, a nucleotide sequence encoding the (R)-linalool synthase is shown as SEQ ID No: 41, a nucleotide sequence encoding the α-pinene synthase is shown as SEQ ID No: 45, a nucleotide sequence encoding the pinene synthase is shown as SEQ ID No: 46, a nucleotide sequence encoding the γ-terpinene synthase is shown as SEQ ID No: 47, a nucleotide sequence encoding the geraniol synthase is shown as SEQ ID No: 48, a nucleotide sequence encoding the (+)-limonene synthase is shown as SEQ ID No: 49, a nucleotide sequence encoding the (−)-limonene synthase is shown as SEQ ID No: 50, a nucleotide sequence encoding the myrcene synthase is shown as SEQ ID No: 51, a nucleotide sequence encoding the β-ocimene synthase is shown as SEQ ID No: 52, a nucleotide sequence encoding the sabinene synthase is shown as SEQ ID No: 53, a GenBank accession number of a nucleotide sequence encoding the farnesyl pyrophosphate synthase is NC_000913 REGION: complement (440202 . . . 441101) (SEQ ID No: 67), a nucleotide sequence encoding the (−)-α-bisabolol synthase is shown as SEQ ID No: 54, a nucleotide sequence encoding the phosphatase is shown as SEQ ID No: 55, a nucleotide sequence encoding the longifolene synthase is shown as SEQ ID No: 56, a nucleotide sequence encoding the valencene synthase is shown as SEQ ID No: 57, a nucleotide sequence encoding the germacrene A synthase is shown as SEQ ID No: 58, a nucleotide sequence encoding the farnesene synthase is shown as SEQ ID No: 59, a nucleotide sequence encoding the patchoulol synthase is shown as SEQ ID No: 60, a nucleotide sequence encoding the pentalenene synthase is shown as SEQ ID No: 61, a nucleotide sequence encoding the α-santalene synthase is shown as SEQ ID No: 62, A nucleotide sequence encoding the isoprene synthase is shown as SEQ ID No: 42, and a nucleotide sequence encoding the isopentenol synthase is shown as SEQ ID No: 43.

In an implementation, an expression vector used includes but is not limited to pBBR1MCS-2, pBbA5c-RFP, or pUCP18.

The disclosure further provides a method for producing terpenoids using the engineered strain of S. marcescens, including: seeding a fermentation medium with the engineered strain of S. marcescens for fermentation at 30° C., 200 rpm for at least 72 h.

In an implementation, the fermentation medium includes glucose (60 g·L⁻¹), Na₂HPO₄: 12H₂O (17.1 g·L⁻¹), KH₂PO₄ (3.0 g·L⁻¹), NaCl (3.0 g·L⁻¹), NH₄Cl (1.0 g·L⁻¹), yeast extract (5.0 g·L⁻¹), citric acid (0.2 g·L⁻¹), MgSO₄ (1.0 mM), CaCl₂) (0.1 mM), thiamine hydrochloride (0.008 g·L⁻¹), D-(+)-biotin (0.008 g·L⁻¹), nicotinic acid (0.008 g·L⁻¹), pyridoxine (0.032 g·L⁻¹), and a trace metal solution (1 mL·L⁻¹).

The trace metal solution includes NaCl (5-15 g·L⁻¹), citric acid (30-50 g·L⁻¹), ZnSO₄·7H₂O (0.5-5 g·L⁻¹), MnSO₄·H₂O (20-40 g·L⁻¹), CuSO₄: 5H₂O (0.1-5 g·L⁻¹), H₃BO₃ (0.1-5 g·L⁻¹), Na₂MoO₄: 2H₂O (0.1-5 g·L⁻¹), FeSO₄·7H₂O (0.5-5 g·L⁻¹), and CoCl₂·6H₂O (0.5-5 g·L⁻¹).

In an implementation, during fermentation, feeding is carried out, and a culture medium used for feeding includes glucose (500-700 g·L⁻¹), KH₂PO₄ (5-15 g·L⁻¹), MgSO₄ (2-5 g·L⁻¹), K₂SO₄ (2-5 g·L⁻¹), Na₂SO₄ (0.1-0.5 g·L⁻¹), thiamine hydrochloride (0.01-0.05 g·L⁻¹), D-(+)-biotin (0.01-0.05 g·L⁻¹), nicotinic acid (0.01-0.05 g·L⁻¹), pyridoxine (0.01-0.05 g·L⁻¹), p-aminobenzoic acid (0.01-0.05 g·L⁻¹), and a trace metal solution (5-15 mL·L⁻¹).

In an implementation, the pH of a fermentation broth is adjusted to 6-8 with ammonia water (25%).

In an implementation, dodecane (10% by volume) is added to the culture medium.

The disclosure further provides an application of the engineered strain of S. marcescens in the production of terpenoids, and examples of the terpenoids include: hemiterpenes (C₅) such as isoprene and isopentenol; monoterpenes (C₁₀) such as 1,8-cineole, linalool, α-pinene, β-pinene, γ-terpinene, geraniol, limonene, myrcene, β-ocimene, and sabinene; and sesquiterpenes (C₁₅) such as bisabolol, farnesol, longifolene, valencene, elemene, farnesene, patchoulol, pentalenene, and santalene.

### Beneficial Effects

The disclosure provides the engineered strain of S. marcescens and method for producing terpenoids. By using the engineered strain of S. marcescens constructed by the disclosure, hemiterpenes, monoterpenes, or sesquiterpenes can be produced by the MVA pathway. By fermentation in a 50 mL shake flask for 72 h, the yield of linalool is 13.01 g. L⁻¹, the yield of isoprene is 12.95 g·L⁻¹, the yield of isopentenol is 11.79 g·L⁻¹, the yield of 1,8-cineole is 11.67 g·L⁻¹, the yield of α-pinene is 10.70 g·L⁻¹, the yield of pinene is 10.15 g·L⁻¹, the yield of γ-terpinene is 11.61 g·L⁻¹, the yield of geraniol is 11.88 g·L⁻¹, the yield of (+)-limonene is 10.89 g·L⁻¹, the yield of (−)-limonene is 11.04 g·L⁻¹, the yield of myrcene is 11.36 g·L⁻¹, the yield of β-ocimene is 11.86 g·L⁻¹, the yield of sabinene is 10.43 g·L⁻¹, the yield of (−)-α-bisabolol is 10.08 g·L⁻¹, the yield of farnesol is 10.33 g·L⁻¹, the yield of longifolene is 10.50 g·L⁻¹, the yield of valencene is 10.10 g·L⁻¹, the yield of β-elemene is 10.72 g·L⁻¹, the yield of farnesene is 10.61 g·L⁻¹, the yield of patchoulol is 10.38 g·L⁻¹, the yield of pentalenene is 10.26 g·L⁻¹, and the yield of α-santalene is 11.40 g·L⁻¹. In a 30 L fermenter, the yield of linalool produced by the engineered strain of S. marcescens is 40.72 g·L⁻¹.

### Preservation of Biological Materials

A strain of S. marcescens HBQA7 which is highly tolerant to organic solvents, named Serratia marcescens HBQA7 in taxonomy, has been deposited at the China Center for Type Culture Collection on Feb. 23, 2023, with the accession number CCTCC NO: M 2023184, at Wuhan University, Wuhan, China.

### BRIEF DESCRIPTION OF FIGURES

FIG. 1 is a schematic diagram of an MVA pathway.

FIG. 2 is a schematic diagram of conversion of isopentenyl pyrophosphate (IPP) and dimethylallyl pyrophosphate (DMAPP) to geranyl pyrophosphate (GPP) and farnesyl pyrophosphate (FPP), as well as synthesis of various terpenoids.

### DETAILED DESCRIPTION

### 1. Strains

Illustrative examples of Serratia marcescens include but are not limited to: the following strains purchased from the American Type Culture Collection (ATCC): Serratia fonticola ATCC 29845, Serratia odorifera ATCC 33077, Serratia plymuthica ATCC 15928, Serratia liquefaciens ATCC 27592, Serratia rubidaea ATCC 19279, Serratia oryzae ATCC 1011, Serratia ureilytica ATCC BAA-2620, Serratia entomophila ATCC 43705, Serratia ficaria ATCC 33105, Serratia marcescens ATCC 13880, and Serratia proteamaculans ATCC 19323; the following strains purchased from German Collection of Microorganisms and Cell Cultures (DSMZ): Serratia symbiotica DSM 23270, Serratia nematodiphila DSM 21420, Serratia quinivorans DSM 4597, and Serratia grimesi DSM 30063; and Serratia marcescens (S. marcescens) HBQA7, preserved at the China Center for Type Culture Collection, with the accession number CCTCC NO: M 2023184. The strain Serratia marcescens HBQA7 has excellent organic solvent-tolerance.

### 2. MVA Pathway

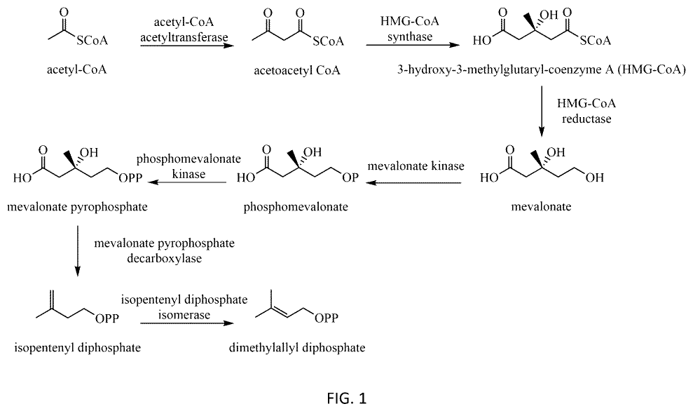

A schematic diagram of the MVA pathway is shown in FIG. 1. Generally, the pathway includes seven steps.

Step 1: Two acetyl-CoA molecules are condensed under enzyme catalysis to produce acetoacetyl COA. A known enzyme for catalysis in this step is acetyl-CoA thiolase (also known as acetyl-CoA acyltransferase).

Step 2: The acetoacetyl CoA molecule and another acetyl-CoA molecule are condensed under enzyme catalysis to produce 3-hydroxy-3-methylglutaryl-CoA (HMG-COA). A known enzyme for catalysis in this step is, for example, HMG-COA synthase.

Step 3: The HMG-CoA produces mevalonate under enzyme catalysis. A known enzyme for catalysis in this step is, for example, HMG-CoA reductase.

Step 4: The mevalonate produces 5-phosphomevalonate under enzyme catalysis. A known enzyme for catalysis in this step is, for example, mevalonate kinase.

Step 5: The mevalonate 5-phosphate produces mevalonate 5-pyrophosphate under enzyme catalysis. A known enzyme for catalysis in this step is, for example, phosphomevalonate kinase.

Step 6: The mevalonate 5-pyrophosphate produces IPP under enzyme catalysis. A known enzyme for catalysis in this step is, for example, mevalonate pyrophosphate decarboxylase.

Step 7: The IPP produces DMAPP under enzyme catalysis. A known enzyme for catalysis in this step is, for example, isopentenyl pyrophosphate isomerase.

3. Hemiterpenes (C₅ compounds)

The hemiterpenes (C₅ compounds) in the disclosure are derived from IPP and DMPAA, and are from one isoprene unit.

### Isoprene

The structure of the isoprene is:

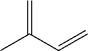

The isoprene is produced through isoprene synthase using IPP as a substrate.

Isopentenol (including 3-methyl-3-buten-1-ol (isoprenol) and 3-methyl-2-buten-1-ol (prenol))

The structure of the 3-methyl-3-buten-1-ol is:

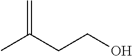

The 3-methyl-3-buten-1-ol is produced through isopentenol synthase using IPP as a substrate.

The structure of 3-methyl-2-buten-1-ol is:

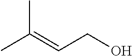

The 3-methyl-2-buten-1-ol is produced through isopentenol synthase using DMAPP as a substrate.

### 4. Monoterpenes (C₁₀ Compounds)

The monoterpenes (C₁₀ compounds) in the disclosure are derived from geranyl pyrophosphate (GPP). GPP is produced by condensation of IPP and DMPAA under enzyme catalysis, and is derived from two isoprene units. A known enzyme for catalysis in this step is, for example, geranyl pyrophosphate synthase.

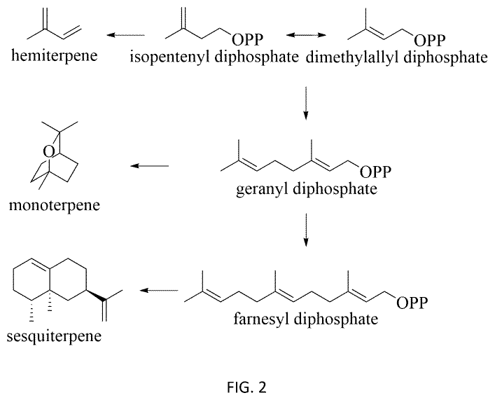

FIG. 2 shows production of GPP from IPP and DMAPP, and GPP may further produce monoterpenes under the catalysis of monoterpene synthase.

Then GPP is converted into various monoterpenes. Illustrative examples of the monoterpenes include but are not limited to:

### 1,8-Cineole

The structure of the 1,8-cineole is:

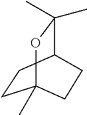

The 1,8-cineole is produced from GPP through 1,8-cineole synthase.

(R)-linalool, the structure of which is:

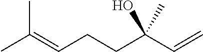

The (R)-linalool is produced from GPP through (R)-linalool synthase.

### (S)-Linalool

The structure of the(S)-linalool is:

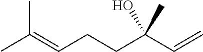

The(S)-linalool is produced from GPP through(S)-linalool synthase.

### α-Pinene

The structure of the α-pinene is:

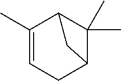

The α-pinene is produced from GPP through α-pinene synthase.

β-Pinene

The structure of the β-pinene is:

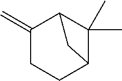

The β-pinene is produced from GPP through β-pinene synthase.

### γ-Terpinene

The structure of the γ-terpinene is:

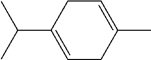

The γ-terpinene is produced from GPP through γ-terpinene synthase.

### Geraniol

The structure of the geraniol is:

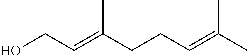

The geraniol is produced from GPP through geraniol synthase.

### Limonene

The structure of (+)-limonene is:

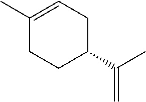

The (+)-limonene is produced from GPP through (+)-limonene synthase.

The structure of (−)-limonene is:

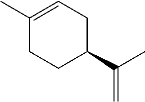

The (−)-limonene is produced from GPP through (−)-limonene synthase.

### Myrcene

The structure of the myrcene is:

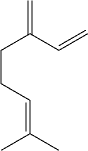

The myrcene is produced from GPP through myrcene synthase.

### β-Ocimene

The structure of the β-ocimene is:

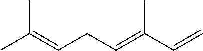

The β-ocimene is produced from GPP through β-ocimene synthase.

### Sabinene

The structure of (+)-sabinene is:

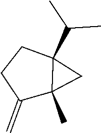

The (+)-sabinene is produced from GPP through (+)-sabinene synthase.

The structure of (−)-sabinene is:

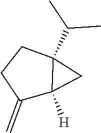

The (−)-sabinene is produced from GPP through (−)-sabinene synthase.

5. Sesquiterpenes (C₁₅ compounds)

The sesquiterpenes (C₁₅ compounds) in the disclosure are derived from farnesyl pyrophosphate (FPP). FPP is produced by condensation of two molecules of IPP and one molecule of DMAPP under enzyme catalysis, and is derived from three isoprene units. A known enzyme for catalysis in this step is, for example, farnesyl pyrophosphate synthase.

FIG. 2 shows production of FPP from IPP and DMAPP, and FPP may further produce sesquiterpenes under the catalysis of sesquiterpene synthase.

Then FPP is converted into various sesquiterpenes. Illustrative examples of the sesquiterpenes include but are not limited to:

### α-Bisabolol

The structure of the α-bisabolol is:

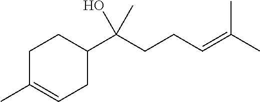 [SMILES: CC(C)=CCCC(C)(O)C1CC=C(C)CC1]

The α-bisabolol is produced from FPP through α-bisabolol synthase.

### Farnesol (Nerolidol)

The structure of the farnesol is:

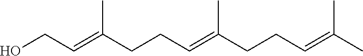 [SMILES: CC(C)=CCC/C(C)=C/CC/C(C)=C/CO]

The farnesol is produced by hydrolysis of FPP through phosphatase.

### Longifolene

The structure of the longifolene is:

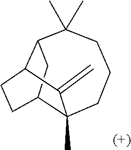 [SMILES: C=C1C2CCC3CC2C(C)(C)CCC[C@@]13C]

The longifolene is produced from FPP through longifolene synthase.

### Valencene

The structure of the valencene is:

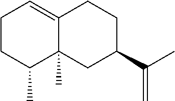

The valencene is produced from FPP through valencene synthase.

### β-Elemene

The structure of the β-elemene is:

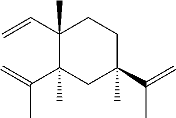 [SMILES: C=C[C@]1(C)CC[C@@](C)(C(=C)C)C[C@@]1(C)C(=C)C]

FPP produces germacrene A under the action of germacrene A synthase, and then the germacrene A is converted into β-elemene through a one-step chemical reaction in vitro.

### Farnesene

The structure of the farnesene is:

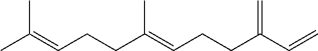

The farnesene is produced from FPP through farnesene synthase.

### Patchoulol

The structure of the patchoulol is:

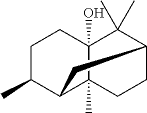 [SMILES: C[C@H]1CC[C@@]2(O)C(C)(C)[C@@H]3CC[C@@]2(C)[C@H]1C3]

The patchoulol is produced from FPP through patchoulol synthase.

### Pentalenene

The structure of the pentalenene is:

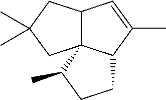

The pentalenene is produced from FPP through pentalenene synthase.

### Santalene

The structure of α-santalene is:

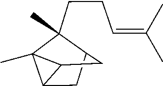

The structure of β-santalene is:

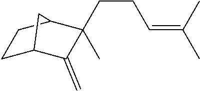

The santalene is produced from FPP through santalene synthase.

### 6. Construction of Terpenoid Production Pathway

The disclosure achieves high-level production of terpenoids in host cells by using the MVA pathway.

The nucleotide sequences may be expressed by one or two vector. For example, an expression vector may contain at least two, three, four, five, six, or all sequences that encode the enzymes used in the MVA pathway. The number of vectors depends on the size of a heterologous sequence and the capacity of the vectors, and mainly depends on the yield of a terpenoid when the terpenoid is expressed in a selected host cell. Or the nucleotide sequences are integrated onto a host genome. Usually, the latter is preferred to ensure stable expression.

The nucleic acid sequences may be codon optimized according to the selected host to achieve high expression in the host. For example, in certain implementations, an acetyl-CoA acetyltransferase gene atoB derived from E. coli is codon optimized with respect to codon preference of S. marcescens.

A nucleic acid may be prepared by various conventional techniques and methods, including but not limited to direct extraction from a cell and synthesis.

The transcription level of a nucleic acid in a host microorganism may be improved by various methods, including but not limited to: (1) enhancing the strength of a promoter of a nucleotide sequence encoding the enzyme; (2) dividing an operon into individual genes controlled by separate promoters; and (3) increasing the copy number of the nucleotide sequence encoding the enzyme to improve the transcription level (for example, introducing additional copies of the nucleotide sequence encoding the enzyme into the genome of the host microorganism, or expressing the nucleotide sequence encoding the enzyme using a plasmid with a high copy number in the host microorganism).

The translation level of the nucleic acid in the host microorganism may be improved by various methods, including but not limited to: (1) modifying the sequence of a ribosome binding site; (2) optimizing the sequence between the ribosome binding site and an initiation codon; and (3) improving the stability of mRNA.

The enzyme activity in the MVA pathway in a host may be changed by various methods, including but not limited to: (1) expressing enzymes with high k_{cat} or low Kₘ; (2) expressing enzymes that are not regulated by positive/negative feedback; and (3) changing the enzyme activity by site-directed mutation or random mutation.

In the implementations of the disclosure, the used expression vectors include but are not limited to pBBR1MCS-2, pBbA5c-RFP, and pUCP18.

In the implementations of the disclosure, a promoter used in the expression vectors is a constitutive promoter. One or more nucleic acid sequences are effectively linked to the constitutive promoter.

In the implementations of the disclosure, suitable constitutive promoters for a host cell include but are not limited to endogenous promoters of S. marcescens and Anderson promoters (BBa_J23100, BBa_J23112, BBa_J23119, and the like).

An expression vector contains one or more selective marker genes for screening and identifying a host cell carrying the expression vector. Examples of suitable selective markers for S. marcescens include but are not limited to ampicillin, kanamycin, chloramphenicol, streptomycin, and spectinomycin resistance.

The expression vector may be introduced into a host cell by various techniques, for example, an electroporation method. The electroporation method uses electrical pulse to form a temporary pore on a cell membrane, allowing a nucleic acid and other substances to enter the cell through the pore.

After transformation, whether a plasmid has been introduced into the host cell may be determined by various methods. One method is to screen the transformed host cell by a resistant selective marker gene, and then validate a single colony by PCR using a specific primer.

The enzyme activity in the pathway may be determined by various methods known in the art. Usually, the enzyme activity may be determined based on the consumption of a substrate or the production of a product. A reaction may be measured in vivo or in vitro. For example, the activity of HMG-COA reductase may be measured in vitro by the consumption of NADH. HMG-COA reductase produces mevalonate using HMG-COA and NADH as substrates. NADH has an absorption peak at 340 nm, and the decrease in the NADH absorbance at 340 nm is measured to detect the activity of the HMG-COA reductase.

TABLE 1
Construction of plasmids

| Plasmid | Carried genes |
|---|---|
| pBBR1MCS-2-MevT | atoB, ERG13, tHMGR (truncated HMG1) |
| pBBR1MCS-2-MevT(co) | atoB, ERG13, tHMGR, codon optimized |
| pBbA5c-MBI | ERG12, ERG8, MVD1, idi |
| pBBR1MCS-2-MTSA | atoB (codon optimized), SamvaS, SamvaA |
| pBBR1MCS-2-MTEF | atoB (codon optimized), EfmvaS, EfmvaE |
| pBBR1MCS-2-MTEF-MevB | atoB (codon optimized), EfmvaS, EfmvaE; |
| | ERG12, ERG8, MVD1 |
| pBBR1MCS-2-MTEF-MBI-trGPPS(co) | atoB (codon optimized), EfmvaS, EfmvaE; |
| | ERG12, ERG8, MVD1, idi; trGPPS(co) |
| pBBR1MCS-2-MTEF-MBI-ispA | atoB (codon optimized), EfmvaS, EfmvaE; |
| | ERG12, ERG8, MVD1, idi; ispA |
| pUCP18-trGPPS(co)-ScLinS | trGPPS(co), ScLinS |
| pBBR1MCS-2-MTEF-MevB-IspS | atoB (codon optimized), EfmvaS, EfmvaE; |
| | ERG12, ERG8, MVD1; IspS |
| pBBR1MCS-2-MTEF-MevB-NudF | atoB (codon optimized), EfmvaS, EfmvaE; |
| | ERG12, ERG8, MVD1; NudF |
| pBBR1MCS-2-MTEF-MBI-trGPPS(co)-ScCinS | t atoB (codon optimized), SamvaS, SamvaA; |
| | ERG12, ERG8, MVD1, idi; rGPPS(co), ScCinS |
| pBBR1MCS-2-MTEF-MBI-trGPPS(co)-ScLinS | atoB (codon optimized), SamvaS, SamvaA; |
| | ERG12, ERG8, MVD1, idi; trGPPS(co), ScLinS |
| pBBR1MCS-2-MTEF-MBI-trGPPS(co)-PtPS | atoB (codon optimized), SamvaS, SamvaA; |
| | ERG12, ERG8, MVD1, idi; trGPPS(co), PtPS |
| pBBR1MCS-2-MTEF-MBI-trGPPS(co)-AgPS | atoB (codon optimized), SamvaS, SamvaA; |
| | ERG12, ERG8, MVD1, idi; trGPPS(co), AgPS |
| pBBR1MCS-2-MTEF-MBI-trGPPS(co)-TvTPS | atoB (codon optimized), SamvaS, SamvaA; |
| | ERG12, ERG8, MVD1, idi; trGPPS(co), TvTPS |
| pBBR1MCS-2-MTEF-MBI-trGPPS(co)-ObGES | atoB (codon optimized), SamvaS, SamvaA; |
| | ERG12, ERG8, MVD1, idi; trGPPS(co), ObGES |
| pBBR1MCS-2-MTEF-MBI-trGPPS(co)-CILS | atoB (codon optimized), SamvaS, SamvaA; |
| | ERG12, ERG8, MVD1, idi; trGPPS(co), CILS |
| pBBR1MCS-2-MTEF-MBI-trGPPS(co)-MsLS | atoB (codon optimized), SamvaS, SamvaA; |
| | ERG12, ERG8, MVD1, idi; trGPPS(co), MsLS |
| pBBR1MCS-2-MTEF-MBI-trGPPS(co)-CsMS | atoB (codon optimized), SamvaS, SamvaA; |
| | ERG12, ERG8, MVD1, idi; trGPPS(co), CsMS |
| pBBR1MCS-2-MTEF-MBI-trGPPS(co)-AtOS | atoB (codon optimized), SamvaS, SamvaA; |
| | ERG12, ERG8, MVD1, idi; trGPPS(co), AtOS |
| pBBR1MCS-2-MTEF-MBI-trGPPS(co)-SpSabS1 | atoB (codon optimized), SamvaS, SamvaA; |
| | ERG12, ERG8, MVD1, idi; trGPPS(co), |
| | SpSabS1 |
| pBBR1MCS-2-MTEF-MBI-ispA-CcBOS | atoB (codon optimized), SamvaS, SamvaA; |
| | ERG12, ERG8, MVD1, idi; ispA, CcBOS |
| pBBR1MCS-2-MTEF-MBI-ispA-PgpB | atoB (codon optimized), SamvaS, SamvaA; |
| | ERG12, ERG8, MVD1, idi; ispA, PgpB |
| pBBR1MCS-2-MTEF-MBI-ispA-PsTPS | atoB (codon optimized), SamvaS, SamvaA; |
| | ERG12, ERG8, MVD1, idi; ispA, PsTPS |
| pBBR1MCS-2-MTEF-MBI-ispA-CnVS | atoB (codon optimized), SamvaS, SamvaA; |
| | ERG12, ERG8, MVD1, idi; ispA, CnVS |
| pBBR1MCS-2-MTEF-MBI-ispA-LsLTC2 | atoB (codon optimized), SamvaS, SamvaA; |
| | ERG12, ERG8, MVD1, idi; ispA, LsLTC2 |
| pBBR1MCS-2-MTEF-MBI-ispA-AaFG | atoB (codon optimized), SamvaS, SamvaA; |
| | ERG12, ERG8, MVD1, idi; ispA, AaFG |
| pBBR1MCS-2-MTEF-MBI-ispA-AaFG | atoB (codon optimized), SamvaS, SamvaA; |
| | ERG12, ERG8, MVD1, idi; ispA, AaFG |
| pBBR1MCS-2-MTEF-MBI-ispA-PSS | atoB (codon optimized), SamvaS, SamvaA; |
| | ERG12, ERG8, MVD1, idi; ispA, PSS |
| pBBR1MCS-2-MTEF-MBI-ispA-SanSyn | atoB (codon optimized), SamvaS, SamvaA; |
| | ERG12, ERG8, MVD1, idi; ispA, SanSyn |

### Example 1

In this example, a method for preparing an expression plasmid is described. The expression plasmid encodes an enzyme in the MVA pathway, and genes encoding the enzyme in the MVA pathway are derived from E. coli and Saccharomyces cerevisiae.

An expression plasmid pBBR1MCS-2-MevT was produced by inserting an MevT operon into a pBBR1MCS-2 vector. The MevT operon contains an atoB gene (GenBank accession number: NC_000913 REGION: 2326109 . . . 2327293) (SEQ ID No: 68) (encoding acetyl-CoA acetyltransferase) derived from E. coli, an ERG13 gene (GenBank accession number: NC_001145 REGION: complement (19060 . . . 20535)) (SEQ ID No: 69) (encoding HMG-COA synthase) derived from S. cerevisiae, and a truncated HMG1 gene (SEQ ID NO:32) (encoding HMG-COA reductase) derived from S. cerevisiae. The genes were directly synthesized into the Kpnl and Xhol restriction enzyme cutting sites of the pBBR1MCS-2 vector in an order of atoB, ERG13, and HMG1, and ribosome binding sites BBa_J61100 (AAAGAGGGGACAA) (SEQ ID No: 70) and BBa_J61106 (AAAGATAGGAGAC) (SEQ ID No: 71) were introduced before ERG13 and HMG1, respectively. Also, an enzyme cutting site Apal was introduced between BBa_J61100 and ERG13 to produce the expression plasmid pBBR1MCS-2-MevT.

An expression plasmid pBBR1MCS-2-MevT (co) was produced by inserting an MevT (co) operon into a pBBR1MCS-2 vector. The MevT (co) operon contains an atoB gene (SEQ ID NO:33) (encoding acetyl-CoA acetyltransferase) derived from E. coli, an ERG13 gene (SEQ ID NO:34) (encoding HMG-CoA synthase) derived from S. cerevisiae, and a truncated HMG1 gene (SEQ ID NO:35) (encoding HMG-COA reductase) derived from S. cerevisiae. The three sequences were codon optimized for expression in S. marcescens. The codon optimized genes were directly synthesized into the Kpnl and Xhol restriction enzyme cutting sites of the pBBR1MCS-2 vector in an order of atoB, ERG13, and HMG1, and ribosome binding sites BBa_J61100 (AAAGAGGGGACAA) (SEQ ID No: 70) and BBa_J61106 (AAAGATAGGAGAC) (SEQ ID No: 71) were introduced before ERG13 and HMG1, respectively. Also, an enzyme cutting site Apal was introduced between BBa_J61100 and ERG13 to produce the expression plasmid pBBR1MCS-2-MevT (co).

An expression plasmid pBbA5c-MBI was produced by inserting an MBI operon into a pBbA5c-RFP vector. The MBI operon contains an ERG12 gene (GenBank accession number: NC_001145 REGION: 684467 . . . 685798) (encoding mevalonate kinase) (SEQ ID No: 63) derived from S. cerevisiae, an ERG8 gene (GenBank accession number: NC_001145 REGION: 712316 . . . 713671) (encoding phosphomevalonate kinase) (SEQ ID No: 64) derived from S. cerevisiae, an MVD1 gene (GenBank accession number: NC_001146 REGION: 701895 . . . 703085) (encoding mevalonate pyrophosphate decarboxylase) (SEQ ID No: 65) derived from S. cerevisiae, and an idi gene (GenBank accession number: NC_000913 REGION: 3033065 . . . 3033613) (encoding isopentenyl pyrophosphate isomerase) (SEQ ID No: 66) derived from E. coli. The four genes were directly synthesized into the EcoRI and BamHl restriction enzyme cutting sites of the pBbA5c-RFP vector in an order of ERG12, ERG8, MVD1, and idi, and ribosome binding sites BBa_J61109 (AAAGACTGGAGAC) (SEQ ID NO:72), BBa_J61112 (AAAGAGGTGACAT) (SEQ ID NO: 73), and BBa_J61115 (AAAGAAGGGATAC) (SEQ ID NO:74) were introduced before ERG8, MVD1, and idi, respectively, to produce the expression plasmid pBbA5c-MBI.

### Example 2

In this example, a method for preparing an expression plasmid is described. The expression plasmid encodes an enzyme in the MVA pathway, and genes encoding the enzyme in the MVA pathway are derived from E. coli and Staphylococcus aureus.

An expression plasmid pBBR1MCS-2-MTSA was derived from an expression plasmid pBBR1MCS-2-MevT (co), and was produced by replacing an EGR13 gene and an HMG1 gene derived from S. cerevisiae with an mvaS gene (SEQ ID NO:36) (encoding HMG-COA synthase) and an mvaA gene (SEQ ID NO:37) (encoding HMG-COA reductase) derived from S. aureus, respectively. The two genes were directly synthesized into the Apal and Xhol restriction enzyme cutting sites of the vector pBBR1MCS-2-MevT (co) in an order of mvaS and mvaA, to produce the expression plasmid pBBR1MCS-2-MTSA.

### Example 3

In this example, a method for preparing an expression plasmid is described. The expression plasmid encodes an enzyme in the MVA pathway, and genes encoding the enzyme in the MVA pathway are derived from E. coli and Enterococcus faecalis.

An expression plasmid pBBR1MCS-2-MTEF was derived from an expression plasmid pBBR1MCS-2-MevT (co), and was produced by replacing an EGR13 gene and an HMG1 gene derived from S. cerevisiae with an mvaS gene (SEQ ID NO:38) (encoding HMG-COA synthase) and an mvaE gene (SEQ ID NO:39) (encoding HMG-COA reductase) derived from E. faecalis, respectively. The two genes were directly synthesized into the Apal and Xhol restriction enzyme cutting sites of the vector pBBR1MCS-2-MevT (co) in an order of mvaS and mvaE, to produce the expression plasmid pBBR1MCS-2-MTEF.

An expression plasmid pBBR1MCS-2-MTEF-MevB was produced by inserting an MevB operon into a pBBR1MCS-2-MTEF vector. The MevB operon contains an ERG12 gene (GenBank accession number: NC_001145 REGION: 684467 . . . 685798) (encoding mevalonate kinase) (SEQ ID No: 63) derived from S. cerevisiae, an ERG8 gene (GenBank accession number: NC 001145 REGION: 712316 . . . 713671) (encoding phosphomevalonate kinase) (SEQ ID No: 64) derived from S. cerevisiae, and an MVD1 gene (GenBank accession number: NC_001146 REGION: 701895 . . . 703085) (encoding mevalonate pyrophosphate decarboxylase) (SEQ ID No: 65) derived from S. cerevisiae. The three genes were directly synthesized into the restriction enzyme cutting sites HindIII and EcoRI of the pBBR1MCS-2-MTEF vector in an order of ERG12, ERG8, and MVD1. A lac UV5 promoter was introduced before ERG12, and ribosome binding sites BBa_J61109 (AAAGACTGGAGAC) (SEQ ID NO:72) and BBa_J61112 (AAAGAGGTGACAT) (SEQ ID NO:73) were introduced before ERG8, MVD1, and idi, respectively, to produce the expression plasmid pBBR1MCS-2-MTEF-MevB.

An expression plasmid pBBR1MCS-2-MTEF-MBI-trGPPS(co) was produced by inserting an MBI operon and a truncated geranyl pyrophosphate synthase gene into a pBBR1MCS-2-MTEF vector. The MBI operon contains an ERG12 gene (GenBank accession number: NC_001145 REGION: 684467 . . . 685798) (encoding mevalonate kinase) (SEQ ID No: 63) derived from S. cerevisiae, an ERG8 gene (GenBank accession number: NC_001145 REGION: 712316 . . . 713671) (encoding phosphomevalonate kinase) (SEQ ID No: 64) derived from S. cerevisiae, an MVD1 gene (GenBank accession number: NC_001146 REGION: 701895 . . . 703085) (encoding mevalonate pyrophosphate decarboxylase) (SEQ ID No: 65) derived from S. cerevisiae, and an idi gene (GenBank accession number: NC_000913 REGION: 3033065 . . . 3033613) (encoding isopentenyl pyrophosphate isomerase) (SEQ ID No: 66) derived from E. coli. The four genes were directly synthesized into the Hindlll and EcoRI restriction enzyme cutting sites of the pBBR1MCS-2-MTEF vector in an order of ERG12, ERG8, MVD1, and idi. A lac UV5 promoter was introduced before ERG12, and ribosome binding sites BBa_J61109 (AAAGACTGGAGAC) (SEQ ID NO:72), BBa_J61112 (AAAGAGGTGACAT) (SEQ ID NO:73), and BBa_J61115 (AAAGAAGGGATAC) (SEQ ID NO:74) were introduced before ERG8, MVD1, and idi, respectively. A geranyl pyrophosphate synthase gene trGPPS derived from truncated Abies grandis was codon optimized for expression in S. marcescens, and has a nucleotide sequence shown as SEQ ID NO:40. The nucleotide sequence was synthesized and inserted into restriction enzyme cutting sites XbaI and SacI of the pBBR1MCS-2-MTEF vector. A lac promoter was introduced before trGPPS, and restriction enzyme cutting site NdeI was introduced after trGPPS, to produce the expression plasmid pBBR1MCS-2-MTEF-MBI-trGPPS(co).

An expression plasmid pBBR1MCS-2-MTEF-MBI-ispA was produced by inserting an MBI operon and ispA into a pBBR1MCS-2-MTEF vector. The MBI operon contains an ERG12 gene (GenBank accession number: NC_001145 REGION: 684467 . . . 685798) (encoding mevalonate kinase) (SEQ ID No: 63) derived from S. cerevisiae, an ERG8 gene (GenBank accession number: NC_001145 REGION: 712316 . . . 713671) (encoding phosphomevalonate kinase) (SEQ ID No: 64) derived from S. cerevisiae, an MVD1 gene (GenBank accession number: NC_001146 REGION: 701895 . . . 703085) (encoding mevalonate pyrophosphate decarboxylase) (SEQ ID No: 65) derived from S. cerevisiae, and an idi gene (GenBank accession number: NC_000913 REGION: 3033065 . . . 3033613) (encoding isopentenyl pyrophosphate isomerase) (SEQ ID No: 66) derived from E. coli. The four genes were directly synthesized into the Hindlll and EcoRI restriction enzyme cutting sites of the pBBR1MCS-2-MTEF vector in an order of ERG12, ERG8, MVD1, and idi. A lac UV5 promoter was introduced before ERG12, and ribosome binding sites BBa_J61109 (AAAGACTGGAGAC) (SEQ ID NO:72), BBa_J61112 (AAAGAGGTGACAT) (SEQ ID NO:73), and BBa_J61115 (AAAGAAGGGATAC) (SEQ ID NO:74) were introduced before ERG8, MVD1, and idi, respectively. An ispA gene (GenBank accession number: NC_000913 REGION: complement (440202 . . . 441101)) (encoding farnesyl pyrophosphate synthase) (SEQ ID No: 67) derived from E. coli was directly synthesized into restriction enzyme cutting sites XbaI and SacI of the pBBR1MCS-2-MTEF vector. A lac promoter was introduced before ispA, and restriction enzyme cutting site NdeI was introduced after ispA, to produce the expression plasmid pBBR1MCS-2-MTEF-MBI-ispA.

### Example 4

In this example, a method for preparing an expression plasmid is described. The expression plasmid encodes terpenoid synthase.

An expression plasmid pUCP18-trGPPS(co)-ScLinS was produced by inserting encoding sequences of geranyl pyrophosphate synthase trGPPS and (R)-linalool synthase into a vector pUCP18. A geranyl pyrophosphate synthase gene trGPPS derived from truncated Abies grandis and an (R)-linalool synthase gene ScLinS derived from Streptomyces clavuligerus were codon optimized for expression in S. marcescens, and have nucleotide sequences shown as SEQ ID NO:40 and SEQ ID NO:41, respectively. The two genes were synthesized and inserted into restriction enzyme cutting sites Hindlll and XbaI of a pUCP18 vector in an order of trGPPS and ScLinS, and a ribosome binding site BBa_J61130 (AAAGAAACGACAT) (SEQ ID NO:75) was introduced before ScLinS, to produce the expression plasmid pUCP18-trGPPS(co)-ScLinS.

An expression plasmid pBBR1MCS-2-MTEF-MevB-IspS was produced by inserting an encoding sequence of isoprene synthase into a vector pBBR1MCS-2-MTEF-MevB. An isoprene synthase gene IspS derived from Populus alba was codon optimized for expression in S. marcescens, and has a nucleotide sequence shown as SEQ ID NO:42. The nucleotide sequence was synthesized and inserted into restriction enzyme cutting sites XbaI and SacI of the pBBR1MCS-2-MTEF-MevB vector, and a lac promoter was introduced before IspS, to produce the expression plasmid pBBR1MCS-2-MTEF-MevB-IspS.

An expression plasmid pBBR1MCS-2-MTEF-MevB-NudF was produced by inserting an encoding sequence of isopentenol synthase into a vector pBBR1MCS-2-MTEF-MevB. An isopentenol synthase gene NudF derived from Bacillus subtilis was codon optimized for expression in S. marcescens, and has a nucleotide sequence shown as SEQ ID NO:43. The nucleotide sequence was synthesized and inserted into restriction enzyme cutting sites XbaI and SacI of the pBBR1MCS-2-MTEF-MevB vector, and a lac promoter was introduced before NudF, to produce the expression plasmid pBBR1MCS-2-MTEF-MevB-NudF.

An expression plasmid pBBR1MCS-2-MTEF-MBI-trGPPS(co)-ScCinS was derived from the expression plasmid pBBR1MCS-2-MTEF-MBI-trGPPS(co). A 1,8-cineole synthase gene ScCinS derived from S. clavuligerus was codon optimized for expression in S. marcescens, and has a nucleotide sequence shown as SEQ ID NO:44. The nucleotide sequence was synthesized and inserted into restriction enzyme cutting sites NdeI and SacI of the pBBR1MCS-2-MTEF-MBI-trGPPS(co) vector, and a ribosome binding site BBa_J61130 (AAAGAAACGACAT) (SEQ ID NO:75) was introduced before ScCinS, to produce the expression plasmid pBBR1MCS-2-MTEF-MBI-trGPPS(co)-ScCinS.

An expression plasmid pBBR1MCS-2-MTEF-MBI-trGPPS(co)-ScLinS was derived from the expression plasmid pBBR1MCS-2-MTEF-MBI-trGPPS(co). An (R)-linalool synthase gene ScLinS derived from S. clavuligerus was codon optimized for expression in S. marcescens, and has a nucleotide sequence shown as SEQ ID NO:41. The nucleotide sequence was synthesized and inserted into restriction enzyme cutting sites NdeI and SacI of the pBBR1MCS-2-MTEF-MBI-trGPPS(co) vector, and a ribosome binding site BBa_J61130 (AAAGAAACGACAT) (SEQ ID NO:75) was introduced before ScLinS, to produce the expression plasmid pBBR1MCS-2-MTEF-MBI-trGPPS(co)-ScLinS.

An expression plasmid pBBR1MCS-2-MTEF-MBI-trGPPS(co)-PtPS was derived from the expression plasmid pBBR1MCS-2-MTEF-MBI-trGPPS(co). An α-pinene synthase gene PtPS derived from Pinus taeda was codon optimized for expression in S. marcescens, and has a nucleotide sequence shown as SEQ ID NO:45. The nucleotide sequence was synthesized and inserted into restriction enzyme cutting sites NdeI and SacI of the pBBR1MCS-2-MTEF-MBI-trGPPS(co) vector, and a ribosome binding site BBa_J61130 (AAAGAAACGACAT) (SEQ ID NO:75) was introduced before PtPS, to produce the expression plasmid pBBR1MCS-2-MTEF-MBI-trGPPS(co)-PtPS.

An expression plasmid pBBR1MCS-2-MTEF-MBI-trGPPS(co)-AgPS was derived from the expression plasmid pBBR1MCS-2-MTEF-MBI-trGPPS(co). A pinene synthase gene AgPS derived from Abies grandis was codon optimized for expression in S. marcescens, and has a nucleotide sequence shown as SEQ ID NO:46. The nucleotide sequence was synthesized and inserted into restriction enzyme cutting sites NdeI and SacI of the pBBR1MCS-2-MTEF-MBI-trGPPS(co) vector, and a ribosome binding site BBa_J61130 (AAAGAAACGACAT) (SEQ ID NO:75) was introduced before AgPS, to produce the expression plasmid pBBR1MCS-2-MTEF-MBI-trGPPS(co)-AgPS.

An expression plasmid pBBR1MCS-2-MTEF-MBI-trGPPS(co)-TvTPS was derived from the expression plasmid pBBR1MCS-2-MTEF-MBI-trGPPS(co). A γ-terpinene synthase gene TvTPS derived from Thymus vulgaris was codon optimized for expression in S. marcescens, and has a nucleotide sequence shown as SEQ ID NO:47. The nucleotide sequence was synthesized and inserted into restriction enzyme cutting sites NdeI and SacI of the pBBR1MCS-2-MTEF-MBI-trGPPS(co) vector, and a ribosome binding site BBa_J61130 (AAAGAAACGACAT) (SEQ ID NO:75) was introduced before TvTPS, to produce the expression plasmid pBBR1MCS-2-MTEF-MBI-trGPPS(co)-TvTPS.

An expression plasmid pBBR1MCS-2-MTEF-MBI-trGPPS(co)-ObGES was derived from the expression plasmid pBBR1MCS-2-MTEF-MBI-trGPPS(co). A geraniol synthase gene ObGES derived from Ocimum basilicum was codon optimized for expression in S. marcescens, and has a nucleotide sequence shown as SEQ ID NO:48. The nucleotide sequence was synthesized and inserted into restriction enzyme cutting sites NdeI and SacI of the pBBR1MCS-2-MTEF-MBI-trGPPS(co) vector, and a ribosome binding site BBa_J61130 (AAAGAAACGACAT) (SEQ ID NO:75) was introduced before ObGES, to produce the expression plasmid pBBR1MCS-2-MTEF-MBI-trGPPS(co)-ObGES.

An expression plasmid pBBR1MCS-2-MTEF-MBI-trGPPS(co)-CILS was derived from the expression plasmid pBBR1MCS-2-MTEF-MBI-trGPPS(co). A (+)-limonene synthase gene CILS derived from Citrus limon was codon optimized for expression in S. marcescens, and has a nucleotide sequence shown as SEQ ID NO:49. The nucleotide sequence was synthesized and inserted into restriction enzyme cutting sites NdeI and SacI of the pBBR1MCS-2-MTEF-MBI-trGPPS(co) vector, and a ribosome binding site BBa_J61130 (AAAGAAACGACAT) (SEQ ID NO:75) was introduced before CILS, to produce the expression plasmid pBBR1MCS-2-MTEF-MBI-trGPPS(co)-CILS.

An expression plasmid pBBR1MCS-2-MTEF-MBI-trGPPS(co)-MsLS was derived from the expression plasmid pBBR1MCS-2-MTEF-MBI-trGPPS(co). A (−)-limonene synthase gene MsLS derived from Mentha spicata was codon optimized for expression in S. marcescens, and has a nucleotide sequence shown as SEQ ID NO:50. The nucleotide sequence was synthesized and inserted into restriction enzyme cutting sites NdeI and SacI of the pBBR1MCS-2-MTEF-MBI-trGPPS(co) vector, and a ribosome binding site BBa_J61130 (AAAGAAACGACAT) (SEQ ID NO:75) was introduced before MsLS, to produce the expression plasmid pBBR1MCS-2-MTEF-MBI-trGPPS(co)-MsLS.

An expression plasmid pBBR1MCS-2-MTEF-MBI-trGPPS(co)-CsMS was derived from the expression plasmid pBBR1MCS-2-MTEF-MBI-trGPPS(co). A myrcene synthase gene CsMS derived from Cannabis sativa was codon optimized for expression in S. marcescens, and has a nucleotide sequence shown as SEQ ID NO:51. The nucleotide sequence was synthesized and inserted into restriction enzyme cutting sites NdeI and SacI of the pBBR1MCS-2-MTEF-MBI-trGPPS(co) vector, and a ribosome binding site BBa_J61130 (AAAGAAACGACAT) (SEQ ID NO:75) was introduced before CsMS, to produce the expression plasmid pBBR1MCS-2-MTEF-MBI-trGPPS(co)-CsMS.

An expression plasmid pBBR1MCS-2-MTEF-MBI-trGPPS(co)-AtOS was derived from the expression plasmid pBBR1MCS-2-MTEF-MBI-trGPPS(co). A β-ocimene synthase gene AtOS derived from Arabidopsis thaliana was codon optimized for expression in S. marcescens, and has a nucleotide sequence shown as SEQ ID NO:52. The nucleotide sequence was synthesized and inserted into restriction enzyme cutting sites NdeI and SacI of the pBBR1MCS-2-MTEF-MBI-trGPPS(co) vector, and a ribosome binding site BBa_J61130 (AAAGAAACGACAT) (SEQ ID NO:75) was introduced before AtOS, to produce the expression plasmid pBBR1MCS-2-MTEF-MBI-trGPPS(co)-AtOS.

An expression plasmid pBBR1MCS-2-MTEF-MBI-trGPPS(co)-SpSabS1 was derived from the expression plasmid pBBR1MCS-2-MTEF-MBI-trGPPS(co). A sabinene synthase gene SpSabS1 derived from Salvia pomifera was codon optimized for expression in S. marcescens, and has a nucleotide sequence shown as SEQ ID NO:53. The nucleotide sequence was synthesized and inserted into restriction enzyme cutting sites NdeI and SacI of the pBBR1MCS-2-MTEF-MBI-trGPPS(co) vector, and a ribosome binding site BBa_J61130 (AAAGAAACGACAT) (SEQ ID NO:75) was introduced before SpSabS1, to produce the expression plasmid pBBR1MCS-2-MTEF-MBI-trGPPS(co)-SpSabS1.

An expression plasmid pBBR1MCS-2-MTEF-MBI-ispA-CcBOS was derived from the expression plasmid pBBR1MCS-2-MTEF-MBI-ispA. A (−)-α-bisabolol synthase gene CcBOS derived from Cynara cardunculus was codon optimized for expression in S. marcescens, and has a nucleotide sequence shown as SEQ ID NO:54. The nucleotide sequence was synthesized and inserted into restriction enzyme cutting sites NdeI and SacI of the pBBR1MCS-2-MTEF-MBI-ispA vector, and a ribosome binding site BBa_J61130 (AAAGAAACGACAT) (SEQ ID NO:75) was introduced before CcBOS, to produce the expression plasmid pBBR1MCS-2-MTEF-MBI-ispA-CcBOS.

An expression plasmid pBBR1MCS-2-MTEF-MBI-ispA-PgpB was derived from the expression plasmid pBBR1MCS-2-MTEF-MBI-ispA. A phosphatase gene PgpB derived from E. coli was codon optimized for expression in S. marcescens, and has a nucleotide sequence shown as SEQ ID NO:55. The nucleotide sequence was synthesized and inserted into restriction enzyme cutting sites NdeI and SacI of the pBBR1MCS-2-MTEF-MBI-ispA vector, and a ribosome binding site BBa_J61130 (AAAGAAACGACAT) (SEQ ID NO:75) was introduced before PgpB, to produce the expression plasmid pBBR1MCS-2-MTEF-MBI-ispA-PgpB.

An expression plasmid pBBR1MCS-2-MTEF-MBI-ispA-PsTPS was derived from the expression plasmid pBBR1MCS-2-MTEF-MBI-ispA. A longifolene synthase gene PsTPS derived from Pinus sylvestris was codon optimized for expression in S. marcescens, and has a nucleotide sequence shown as SEQ ID NO:56. The nucleotide sequence was synthesized and inserted into restriction enzyme cutting sites NdeI and SacI of the pBBR1MCS-2-MTEF-MBI-ispA vector, and a ribosome binding site BBa_J61130 (AAAGAAACGACAT) (SEQ ID NO:75) was introduced before PsTPS, to produce the expression plasmid pBBR1MCS-2-MTEF-MBI-ispA-PsTPS.

An expression plasmid pBBR1MCS-2-MTEF-MBI-ispA-CnVS was derived from the expression plasmid pBBR1MCS-2-MTEF-MBI-ispA. A valencene synthase gene CnVS derived from Callitropsis nootkatensis was codon optimized for expression in S. marcescens, and has a nucleotide sequence shown as SEQ ID NO:57. The nucleotide sequence was synthesized and inserted into restriction enzyme cutting sites NdeI and SacI of the pBBR1MCS-2-MTEF-MBI-ispA vector, and a ribosome binding site BBa_J61130 (AAAGAAACGACAT) (SEQ ID NO:75) was introduced before CnVS, to produce the expression plasmid pBBR1MCS-2-MTEF-MBI-ispA-CnVS.

An expression plasmid pBBR1MCS-2-MTEF-MBI-ispA-LsLTC2 was derived from the expression plasmid pBBR1MCS-2-MTEF-MBI-ispA. A germacrene A synthase gene LsLTC2 derived from Lactuca sativa was codon optimized for expression in S. marcescens, and has a nucleotide sequence shown as SEQ ID NO:58. The nucleotide sequence was synthesized and inserted into restriction enzyme cutting sites NdeI and SacI of the pBBR1MCS-2-MTEF-MBI-ispA vector, and a ribosome binding site BBa_J61130 (AAAGAAACGACAT) (SEQ ID NO:75) was introduced before LsLTC2, to produce the expression plasmid pBBR1MCS-2-MTEF-MBI-ispA-LsLTC2.

An expression plasmid pBBR1MCS-2-MTEF-MBI-ispA-AaFG was derived from the expression plasmid pBBR1MCS-2-MTEF-MBI-ispA. A farnesene synthase gene AaFG derived from Artemisia annua was codon optimized for expression in S. marcescens, and has a nucleotide sequence shown as SEQ ID NO:59. The nucleotide sequence was synthesized and inserted into restriction enzyme cutting sites NdeI and SacI of the pBBR1MCS-2-MTEF-MBI-ispA vector, and a ribosome binding site BBa_J61130 (AAAGAAACGACAT) (SEQ ID NO:75) was introduced before AaFG, to produce the expression plasmid pBBR1MCS-2-MTEF-MBI-ispA-AaFG.

An expression plasmid pBBR1MCS-2-MTEF-MBI-ispA-PcPTS was derived from the expression plasmid pBBR1MCS-2-MTEF-MBI-ispA. A patchoulol synthase gene PcPTS derived from Pogostemon cablin was codon optimized for expression in S. marcescens, and has a nucleotide sequence shown as SEQ ID NO:60. The nucleotide sequence was synthesized and inserted into restriction enzyme cutting sites NdeI and SacI of the pBBR1MCS-2-MTEF-MBI-ispA vector, and a ribosome binding site BBa_J61130 (AAAGAAACGACAT) (SEQ ID NO:75) was introduced before PcPTS, to produce the expression plasmid pBBR1MCS-2-MTEF-MBI-ispA-PcPTS.

An expression plasmid pBBR1MCS-2-MTEF-MBI-ispA-PSS was derived from the expression plasmid pBBR1MCS-2-MTEF-MBI-ispA. A pentalenene synthase gene PSS derived from Streptomyces sp. was codon optimized for expression in S. marcescens, and has a nucleotide sequence shown as SEQ ID NO:61. The nucleotide sequence was synthesized and inserted into restriction enzyme cutting sites NdeI and SacI of the pBBR1MCS-2-MTEF-MBI-ispA vector, and a ribosome binding site BBa_J61130 (AAAGAAACGACAT) (SEQ ID NO:75) was introduced before PSS, to produce the expression plasmid pBBR1MCS-2-MTEF-MBI-ispA-PSS.

An expression plasmid pBBR1MCS-2-MTEF-MBI-ispA-SanSyn was derived from the expression plasmid pBBR1MCS-2-MTEF-MBI-ispA. An α-santalene synthase gene SanSyn derived from Clausena lansium was codon optimized for expression in S. marcescens, and has a nucleotide sequence shown as SEQ ID NO:62. The nucleotide sequence was synthesized and inserted into restriction enzyme cutting sites NdeI and SacI of the pBBR1MCS-2-MTEF-MBI-ispA vector, and a ribosome binding site BBa_J61130 (AAAGAAACGACAT) (SEQ ID NO:75) was introduced before SanSyn, to produce the expression plasmid pBBR1MCS-2-MTEF-MBI-ispA-SanSyn.

### Example 5

In this example, production of S. marcescens in which an expression plasmid is introduced is described.

As shown in Table 1, a cell of S. marcescens is transformed by one or more expression plasmids. Taking construction of a strain S01 as an example, a plasmid pBBR1MCS-2-MevT is introduced into S. marcescens HBQA7 to obtain the strain S01.

TABLE 2
Construction of strains

| Name | | |
|---|---|---|
| of | Parent strain of | |
| strain | S. marcescens | Expression plasmid |
| S01 | Serratia marcescens | pBBR1MCS-2-MevT |
| | HBQA7 | |
| S02 | Serratia marcescens | pBBR1MCS-2-MevT(co) |
| | HBQA7 | |
| S03 | Serratia marcescens | pBBR1MCS-2-MTSA |
| | HBQA7 | |
| S04 | Serratia marcescens | pBBR1MCS-2-MTEF |
| | HBQA7 | |
| S05 | Serratia marcescens | pBBR1MCS-2-MTEF |
| | HBQA7 | pBbA5c-MBI |
| | | pUCP18-trGPPS(co)-ScLinS |
| S06 | Serratia marcescens | pBBR1MCS-2-MTEF-MBI-trGPPS(co)-ScLinS |
| | HBQA7 | |
| S07 | Serratia fonticola ATCC | pBBR1MCS-2-MTEF-MBI-trGPPS(co)-ScLinS |
| | 29845 | |
| S08 | Serratia odorifera ATCC | pBBR1MCS-2-MTEF-MBI-trGPPS(co)-ScLinS |
| | 33077 | |
| S09 | Serratia plymuthica ATCC | pBBR1MCS-2-MTEF-MBI-trGPPS(co)-ScLinS |
| | 15928 | |
| S10 | Serratia liquefaciens ATCC | pBBR1MCS-2-MTEF-MBI-trGPPS(co)-ScLinS |
| | 27592 | |
| S11 | Serratia rubidaea ATCC | pBBR1MCS-2-MTEF-MBI-trGPPS(co)-ScLinS |
| | 19279 | |
| S12 | Serratia oryzae ATCC 1011 | pBBR1MCS-2-MTEF-MBI-trGPPS(co)-ScLinS |
| S13 | Serratia ureilytica ATCC | pBBR1MCS-2-MTEF-MBI-trGPPS(co)-ScLinS |
| | BAA-2620 | |
| S14 | Serratia entomophila ATCC | pBBR1MCS-2-MTEF-MBI-trGPPS(co)-ScLinS |
| | 43705 | |
| S15 | Serratia ficaria ATCC 33105 | pBBR1MCS-2-MTEF-MBI-trGPPS(co)-ScLinS |
| S16 | Serratia marcescens ATCC | pBBR1MCS-2-MTEF-MBI-trGPPS(co)-ScLinS |
| | 13880 | |
| S17 | Serratia proteamaculans | pBBR1MCS-2-MTEF-MBI-trGPPS(co)-ScLinS |
| | ATCC 19323 | |
| S18 | Serratia symbiotica DSM | pBBR1MCS-2-MTEF-MBI-trGPPS(co)-ScLinS |
| | 23270 | |
| S19 | Serratia nematodiphila | pBBR1MCS-2-MTEF-MBI-trGPPS(co)-ScLinS |
| | DSM 21420 | |
| S20 | Serratia quinivorans DSM | pBBR1MCS-2-MTEF-MBI-trGPPS(co)-ScLinS |
| | 4597 | |
| S21 | Serratia grimesi DSM | pBBR1MCS-2-MTEF-MBI-trGPPS(co)-ScLinS |
| | 30063 | |
| S22 | Serratia marcescens | pBBR1MCS-2-MTEF-MevB-IspS |
| | HBQA7 | |
| S23 | Serratia fonticola ATCC | pBBR1MCS-2-MTEF-MBI-trGPPS(co)-IspS |
| | 29845 | |
| S24 | Serratia odorifera ATCC | pBBR1MCS-2-MTEF-MBI-trGPPS(co)-IspS |
| | 33077 | |
| S25 | Serratia plymuthica ATCC | pBBR1MCS-2-MTEF-MBI-trGPPS(co)-IspS |
| | 15928 | |
| S26 | Serratia liquefaciens ATCC | pBBR1MCS-2-MTEF-MBI-trGPPS(co)-IspS |
| | 27592 | |
| S27 | Serratia rubidaea ATCC | pBBR1MCS-2-MTEF-MBI-trGPPS(co)-IspS |
| | 19279 | |
| S28 | Serratia oryzae ATCC 1011 | pBBR1MCS-2-MTEF-MBI-trGPPS(co)-IspS |
| S29 | Serratia ureilytica ATCC | pBBR1MCS-2-MTEF-MBI-trGPPS(co)-IspS |
| | BAA-2620 | |
| S30 | Serratia entomophila ATCC | pBBR1MCS-2-MTEF-MBI-trGPPS(co)-IspS |
| | 43705 | |
| S31 | Serratia ficaria ATCC 33105 | pBBR1MCS-2-MTEF-MBI-trGPPS(co)-IspS |
| S32 | Serratia marcescens ATCC | pBBR1MCS-2-MTEF-MBI-trGPPS(co)-IspS |
| | 13880 | |
| S33 | Serratia proteamaculans | pBBR1MCS-2-MTEF-MBI-trGPPS(co)-IspS |
| | ATCC 19323 | |
| S34 | Serratia symbiotica DSM | pBBR1MCS-2-MTEF-MBI-trGPPS(co)-IspS |
| | 23270 | |
| S35 | Serratia nematodiphila | pBBR1MCS-2-MTEF-MBI-trGPPS(co)-IspS |
| | DSM 21420 | |
| S36 | Serratia quinivorans DSM | pBBR1MCS-2-MTEF-MBI-trGPPS(co)-IspS |
| | 4597 | |
| S37 | Serratia grimesi DSM | pBBR1MCS-2-MTEF-MBI-trGPPS(co)-IspS |
| | 30063 | |
| S38 | Serratia marcescens | pBBR1MCS-2-MTEF-MevB-NudF |
| | HBQA7 | |
| S39 | Serratia fonticola ATCC | pBBR1MCS-2-MTEF-MBI-trGPPS(co)-NudF |
| | 29845 | |
| S40 | Serratia odorifera ATCC | pBBR1MCS-2-MTEF-MBI-trGPPS(co)-NudF |
| | 33077 | |
| S41 | Serratia plymuthica ATCC | pBBR1MCS-2-MTEF-MBI-trGPPS(co)-NudF |
| | 15928 | |
| S42 | Serratia liquefaciens ATCC | pBBR1MCS-2-MTEF-MBI-trGPPS(co)-NudF |
| | 27592 | |
| S43 | Serratia rubidaea ATCC | pBBR1MCS-2-MTEF-MBI-trGPPS(co)-NudF |
| | 19279 | |
| S44 | Serratia oryzae ATCC 1011 | pBBR1MCS-2-MTEF-MBI-trGPPS(co)-NudF |
| S45 | Serratia ureilytica ATCC | pBBR1MCS-2-MTEF-MBI-trGPPS(co)-NudF |
| | BAA-2620 | |
| S46 | Serratia entomophila ATCC | pBBR1MCS-2-MTEF-MBI-trGPPS(co)-NudF |
| | 43705 | |
| S47 | Serratia ficaria ATCC 33105 | pBBR1MCS-2-MTEF-MBI-trGPPS(co)-NudF |
| S48 | Serratia marcescens ATCC | pBBR1MCS-2-MTEF-MBI-trGPPS(co)-NudF |
| | 13880 | |
| S49 | Serratia proteamaculans | pBBR1MCS-2-MTEF-MBI-trGPPS(co)-NudF |
| | ATCC 19323 | |
| S50 | Serratia symbiotica DSM | pBBR1MCS-2-MTEF-MBI-trGPPS(co)-NudF |
| | 23270 | |
| S51 | Serratia nematodiphila | pBBR1MCS-2-MTEF-MBI-trGPPS(co)-NudF |
| | DSM 21420 | |
| S52 | Serratia quinivorans DSM | pBBR1MCS-2-MTEF-MBI-trGPPS(co)-NudF |
| | 4597 | |
| S53 | Serratia grimesi DSM | pBBR1MCS-2-MTEF-MBI-trGPPS(co)-NudF |
| | 30063 | |
| S54 | Serratia marcescens | pBBR1MCS-2-MTEF-MBI-trGPPS(co)-ScCinS |
| | HBQA7 | |
| S55 | Serratia fonticola ATCC | pBBR1MCS-2-MTEF-MBI-trGPPS(co)-ScCinS |
| | 29845 | |
| S56 | Serratia odorifera ATCC | pBBR1MCS-2-MTEF-MBI-trGPPS(co)-ScCinS |
| | 33077 | |
| S57 | Serratia plymuthica ATCC | pBBR1MCS-2-MTEF-MBI-trGPPS(co)-ScCinS |
| | 15928 | |
| S58 | Serratia liquefaciens ATCC | pBBR1MCS-2-MTEF-MBI-trGPPS(co)-ScCinS |
| | 27592 | |
| S59 | Serratia rubidaea ATCC | pBBR1MCS-2-MTEF-MBI-trGPPS(co)-ScCinS |
| | 19279 | |
| S60 | Serratia oryzae ATCC 1011 | pBBR1MCS-2-MTEF-MBI-trGPPS(co)-ScCinS |
| S61 | Serratia ureilytica ATCC | pBBR1MCS-2-MTEF-MBI-trGPPS(co)-ScCinS |
| | BAA-2620 | |
| S62 | Serratia entomophila ATCC | pBBR1MCS-2-MTEF-MBI-trGPPS(co)-ScCinS |
| | 43705 | |
| S63 | Serratia ficaria ATCC 33105 | pBBR1MCS-2-MTEF-MBI-trGPPS(co)-ScCinS |
| S64 | Serratia marcescens ATCC | pBBR1MCS-2-MTEF-MBI-trGPPS(co)-ScCinS |
| | 13880 | |
| S65 | Serratia proteamaculans | pBBR1MCS-2-MTEF-MBI-trGPPS(co)-ScCinS |
| | ATCC 19323 | |
| S66 | Serratia symbiotica DSM | pBBR1MCS-2-MTEF-MBI-trGPPS(co)-ScCinS |
| | 23270 | |
| S67 | Serratia nematodiphila | pBBR1MCS-2-MTEF-MBI-trGPPS(co)-ScCinS |
| | DSM 21420 | |
| S68 | Serratia quinivorans DSM | pBBR1MCS-2-MTEF-MBI-trGPPS(co)-ScCinS |
| | 4597 | |
| S69 | Serratia grimesi DSM | pBBR1MCS-2-MTEF-MBI-trGPPS(co)-ScCinS |
| | 30063 | |
| S70 | Serratia marcescens | pBBR1MCS-2-MTEF-MBI-trGPPS(co)-PtPS |
| | HBQA7 | |
| S71 | Serratia fonticola ATCC | pBBR1MCS-2-MTEF-MBI-trGPPS(co)-PtPS |
| | 29845 | |
| S72 | Serratia odorifera ATCC | pBBR1MCS-2-MTEF-MBI-trGPPS(co)-PtPS |
| | 33077 | |
| S73 | Serratia plymuthica ATCC | pBBR1MCS-2-MTEF-MBI-trGPPS(co)-PtPS |
| | 15928 | |
| S74 | Serratia liquefaciens ATCC | pBBR1MCS-2-MTEF-MBI-trGPPS(co)-PtPS |
| | 27592 | |
| S75 | Serratia rubidaea ATCC | pBBR1MCS-2-MTEF-MBI-trGPPS(co)-PtPS |
| | 19279 | |
| S76 | Serratia oryzae ATCC 1011 | pBBR1MCS-2-MTEF-MBI-trGPPS(co)-PtPS |
| S77 | Serratia ureilytica ATCC | pBBR1MCS-2-MTEF-MBI-trGPPS(co)-PtPS |
| | BAA-2620 | |
| S78 | Serratia entomophila ATCC | pBBR1MCS-2-MTEF-MBI-trGPPS(co)-PtPS |
| | 43705 | |
| S79 | Serratia ficaria ATCC 33105 | pBBR1MCS-2-MTEF-MBI-trGPPS(co)-PtPS |
| S80 | Serratia marcescens ATCC | pBBR1MCS-2-MTEF-MBI-trGPPS(co)-PtPS |
| | 13880 | |
| S81 | Serratia proteamaculans | pBBR1MCS-2-MTEF-MBI-trGPPS(co)-PtPS |
| | ATCC 19323 | |
| S82 | Serratia symbiotica DSM | pBBR1MCS-2-MTEF-MBI-trGPPS(co)-PtPS |
| | 23270 | |
| S83 | Serratia nematodiphila | pBBR1MCS-2-MTEF-MBI-trGPPS(co)-PtPS |
| | DSM 21420 | |
| S84 | Serratia quinivorans DSM | pBBR1MCS-2-MTEF-MBI-trGPPS(co)-PtPS |
| | 4597 | |
| S85 | Serratia grimesi DSM | pBBR1MCS-2-MTEF-MBI-trGPPS(co)-PtPS |
| | 30063 | |
| S86 | Serratia marcescens | pBBR1MCS-2-MTEF-MBI-trGPPS(co)-AgPS |
| | HBQA7 | |
| S87 | Serratia marcescens | pBBR1MCS-2-MTEF-MBI-trGPPS(co)-TvTPS |
| | HBQA7 | |
| S88 | Serratia marcescens | pBBR1MCS-2-MTEF-MBI-trGPPS(co)-ObGES |
| | HBQA7 | |
| S89 | Serratia marcescens | pBBR1MCS-2-MTEF-MBI-trGPPS(co)-CILS |
| | HBQA7 | |
| S90 | Serratia marcescens | pBBR1MCS-2-MTEF-MBI-trGPPS(co)-MsLS |
| | HBQA7 | |
| S91 | Serratia marcescens | pBBR1MCS-2-MTEF-MBI-trGPPS(co)-CsMS |
| | HBQA7 | |
| S92 | Serratia marcescens | pBBR1MCS-2-MTEF-MBI-trGPPS(co)-AtOS |
| | HBQA7 | |
| S93 | Serratia marcescens | pBBR1MCS-2-MTEF-MBI-trGPPS(co)-SpSabS1 |
| | HBQA7 | |
| S94 | Serratia marcescens | pBBR1MCS-2-MTEF-MBI-ispA-CcBOS |
| | HBQA7 | |
| S95 | Serratia fonticola ATCC | pBBR1MCS-2-MTEF-MBI-ispA-CcBOS |
| | 29845 | |
| S96 | Serratia odorifera ATCC | pBBR1MCS-2-MTEF-MBI-ispA-CcBOS |
| | 33077 | |
| S97 | Serratia plymuthica ATCC | pBBR1MCS-2-MTEF-MBI-ispA-CcBOS |
| | 15928 | |
| S98 | Serratia liquefaciens ATCC | pBBR1MCS-2-MTEF-MBI-ispA-CcBOS |
| | 27592 | |
| S99 | Serratia rubidaea ATCC | pBBR1MCS-2-MTEF-MBI-ispA-CcBOS |
| | 19279 | |
| S100 | Serratia oryzae ATCC 1011 | pBBR1MCS-2-MTEF-MBI-ispA-CcBOS |
| S101 | Serratia ureilytica ATCC | pBBR1MCS-2-MTEF-MBI-ispA-CcBOS |
| | BAA-2620 | |
| S102 | Serratia entomophila ATCC | pBBR1MCS-2-MTEF-MBI-ispA-CcBOS |
| | 43705 | |
| S103 | Serratia ficaria ATCC 33105 | pBBR1MCS-2-MTEF-MBI-ispA-CcBOS |
| S104 | Serratia marcescens ATCC | pBBR1MCS-2-MTEF-MBI-ispA-CcBOS |
| | 13880 | |
| S105 | Serratia proteamaculans | pBBR1MCS-2-MTEF-MBI-ispA-CcBOS |
| | ATCC 19323 | |
| S106 | Serratia symbiotica DSM | pBBR1MCS-2-MTEF-MBI-ispA-CcBOS |
| | 23270 | |
| S107 | Serratia nematodiphila | pBBR1MCS-2-MTEF-MBI-ispA-CcBOS |
| | DSM 21420 | |
| S108 | Serratia quinivorans DSM | pBBR1MCS-2-MTEF-MBI-ispA-CcBOS |
| | 4597 | |
| S109 | Serratia grimesi DSM | pBBR1MCS-2-MTEF-MBI-ispA-CcBOS |
| | 30063 | |
| S110 | Serratia marcescens | pBBR1MCS-2-MTEF-MBI-ispA-PgpB |
| | HBQA7 | |
| S111 | Serratia fonticola ATCC | pBBR1MCS-2-MTEF-MBI-ispA-PgpB |
| | 29845 | |
| S112 | Serratia odorifera ATCC | pBBR1MCS-2-MTEF-MBI-ispA-PgpB |
| | 33077 | |
| S113 | Serratia plymuthica ATCC | pBBR1MCS-2-MTEF-MBI-ispA-PgpB |
| | 15928 | |
| S114 | Serratia liquefaciens ATCC | pBBR1MCS-2-MTEF-MBI-ispA-PgpB |
| | 27592 | |
| S115 | Serratia rubidaea ATCC | pBBR1MCS-2-MTEF-MBI-ispA-PgpB |
| | 19279 | |
| S116 | Serratia oryzae ATCC 1011 | pBBR1MCS-2-MTEF-MBI-ispA-PgpB |
| S117 | Serratia ureilytica ATCC | pBBR1MCS-2-MTEF-MBI-ispA-PgpB |
| | BAA-2620 | |
| S118 | Serratia entomophila ATCC | pBBR1MCS-2-MTEF-MBI-ispA-PgpB |
| | 43705 | |
| S119 | Serratia ficaria ATCC 33105 | pBBR1MCS-2-MTEF-MBI-ispA-PgpB |
| S120 | Serratia marcescens ATCC | pBBR1MCS-2-MTEF-MBI-ispA-PgpB |
| | 13880 | |
| S121 | Serratia proteamaculans | pBBR1MCS-2-MTEF-MBI-ispA-PgpB |
| | ATCC 19323 | |
| S122 | Serratia symbiotica DSM | pBBR1MCS-2-MTEF-MBI-ispA-PgpB |
| | 23270 | |
| S123 | Serratia nematodiphila | pBBR1MCS-2-MTEF-MBI-ispA-PgpB |
| | DSM 21420 | |
| S124 | Serratia quinivorans DSM | pBBR1MCS-2-MTEF-MBI-ispA-PgpB |
| | 4597 | |
| S125 | Serratia grimesi DSM | pBBR1MCS-2-MTEF-MBI-ispA-PgpB |
| | 30063 | |
| S126 | Serratia marcescens | pBBR1MCS-2-MTEF-MBI-ispA-PsTPS |
| | HBQA7 | |
| S127 | Serratia fonticola ATCC | pBBR1MCS-2-MTEF-MBI-ispA-PsTPS |
| | 29845 | |
| S128 | Serratia odorifera ATCC | pBBR1MCS-2-MTEF-MBI-ispA-PsTPS |
| | 33077 | |
| S129 | Serratia plymuthica ATCC | pBBR1MCS-2-MTEF-MBI-ispA-PsTPS |
| | 15928 | |
| S130 | Serratia liquefaciens ATCC | pBBR1MCS-2-MTEF-MBI-ispA-PsTPS |
| | 27592 | |
| S131 | Serratia rubidaea ATCC | pBBR1MCS-2-MTEF-MBI-ispA-PsTPS |
| | 19279 | |
| S132 | Serratia oryzae ATCC 1011 | pBBR1MCS-2-MTEF-MBI-ispA-PsTPS |
| S133 | Serratia ureilytica ATCC | pBBR1MCS-2-MTEF-MBI-ispA-PsTPS |
| | BAA-2620 | |
| S134 | Serratia entomophila ATCC | pBBR1MCS-2-MTEF-MBI-ispA-PsTPS |
| | 43705 | |
| S135 | Serratia ficaria ATCC 33105 | pBBR1MCS-2-MTEF-MBI-ispA-PsTPS |
| S136 | Serratia marcescens ATCC | pBBR1MCS-2-MTEF-MBI-ispA-PsTPS |
| | 13880 | |
| S137 | Serratia proteamaculans | pBBR1MCS-2-MTEF-MBI-ispA-PsTPS |
| | ATCC 19323 | |
| S138 | Serratia symbiotica DSM | pBBR1MCS-2-MTEF-MBI-ispA-PsTPS |
| | 23270 | |
| S139 | Serratia nematodiphila | pBBR1MCS-2-MTEF-MBI-ispA-PsTPS |
| | DSM 21420 | |
| S140 | Serratia quinivorans DSM | pBBR1MCS-2-MTEF-MBI-ispA-PsTPS |
| | 4597 | |
| S141 | Serratia grimesi DSM | pBBR1MCS-2-MTEF-MBI-ispA-PsTPS |
| | 30063 | |
| S142 | Serratia marcescens | pBBR1MCS-2-MTEF-MBI-ispA-CnVS |
| | HBQA7 | |
| S143 | Serratia marcescens | pBBR1MCS-2-MTEF-MBI-ispA-LsLTC2 |
| | HBQA7 | |
| S144 | Serratia marcescens | pBBR1MCS-2-MTEF-MBI-ispA-AaFG |
| | HBQA7 | |
| S145 | Serratia marcescens | pBBR1MCS-2-MTEF-MBI-ispA-PcPTS |
| | HBQA7 | |
| S146 | Serratia marcescens | pBBR1MCS-2-MTEF-MBI-ispA-PSS |
| | HBQA7 | |
| S147 | Serratia marcescens | pBBR1MCS-2-MTEF-MBI-ispA-SanSyn |
| | HBQA7 | |

S. marcescens in which the plasmid is successfully introduced was screened on an LB plate containing an antibiotic. A single colony was inoculated into 3 mL of a liquid LB culture medium containing an antibiotic. All the strains were cultured overnight at 30° C., 200 rpm. 500 μL of a bacterial culture solution was taken and put in a sterilized cryotube containing 500 μL of glycerol (60%) for preservation at −80° C.

### Example 6

This example demonstrates that HMGS and HMGR derived from E. faecalis are superior to HMGS and HMGR derived from S. aureus and S. cerevisiae.

Strains S01, S02, S03, and S04 constructed in Example 5 were streaked and cultured on LB solid media. A single colony of each of the strains S01, S02, S03, and S04 was picked and cultured in 3 mL of a liquid LB culture medium for 12 h, and inoculated into a 250 mL shake flask containing 50 ml of a culture medium at an inoculum concentration of 2% (by volume). The culture medium used is an MVA fermentation medium containing glucose (60 g·L 1), and the strains were cultured at 30° C., 120 rpm for 72 h. The MVA fermentation medium further contains the following substances: Na₂HPO₄·12H₂O (17.1 g·L⁻¹), KH₂PO₄ (3.0 g·L⁻¹), NaCl (3.0 g·L⁻¹), NH₄Cl (1.0 g·L⁻¹), yeast extract (5.0 g·L⁻¹), citric acid (0.2 g·L⁻¹), MgSO₄ (1.0 mM), CaCl₂) (0.1 mM), thiamine hydrochloride (0.008 g·L⁻¹), D-(+)-biotin (0.008 g·L⁻¹), nicotinic acid (0.008 g·L⁻¹), pyridoxine (0.032 g·L⁻¹), and a trace metal solution (1 mL·L⁻¹). The trace metal solution contains the following substances: NaCl (10 g·L⁻¹), citric acid (40 g·L⁻¹), ZnSO₄·7H₂O (1.0 g·L⁻¹), MnSO₄·H₂O (30 g·L⁻¹), CuSO₄·5H₂O (0.1 g·L⁻¹), H₃BO₃ (0.1 g·L⁻¹), Na₂MoO₄: 2H₂O (0.1 g·L⁻¹), FeSO₄: 7H₂O (1.0 g·L⁻¹), and CoCl₂·6H₂O (1.0 g·L⁻¹). An OD value was measured at 600 nm using a spectrophotometer. The OD values of the fermentation broths of the strains S01, S02, S03, and S04 were 10.61, 10.82, 10.20, and 10.09, respectively. The fermentation broths were centrifuged at 12,000 rpm for 10 min, and the supernatant was taken for detection of the yield of mevalonate. The mevalonate was detected by high-performance liquid chromatography (HPLC). The yields of mevalonate of the strains S01, S02, S03, and S04 were 20.63 g·L⁻¹, 21.82 g·L⁻¹, 23.50 g·L⁻¹, and 24.01 g·L⁻¹, respectively. Compared to the strains S01, S02, and S03, the strain S04 expressing the HMGS and HMGR derived from E. faecalis has a higher yield of mevalonate, indicating that the HMGS and HMGR derived from E. faecalis are superior to the HMGS and HMGR derived from S. aureus and S. cerevisiae.

### Example 7

This example demonstrates that single plasmid expression is superior to multi-plasmid expression.

Strains S05 and S06 were streaked and cultured on LB solid media. A single colony of each of the strains S05 and S06 was picked, and inoculated into 3 mL of a 2YT culture medium (peptone (16.0 g·L⁻¹), NaCl (5.0 g·L⁻¹), and yeast extract (10.0 g·L⁻¹)). The strains were cultured overnight at 30° C., 200 rpm. Then, the strains were inoculated into 50 ml of a fresh 2YT culture medium to which glucose (60 g·L⁻¹) at an inoculum concentration of 2% (by volume). To collect linalool, dodecane (10% by volume) was added to the culture medium. Linalool would enter the dodecane on top of the culture medium, and the linalool in the dodecane is detected by gas chromatography. The strains were cultured at 30° C., 120 rpm for 72 h. An OD value was measured at 600 nm using a spectrophotometer. The linalool in the dodecane was detected by gas chromatography. The OD values of the strains S05 and S06 were 6.27 and 6.64, respectively, and the yields of linalool were 9.21 g·L⁻¹ and 13.01 g·L⁻¹, respectively. Compared to the strain S05, the strain S06 has a higher yield of linalool, indicating that single plasmid expression is superior to multi-plasmid expression.

### Example 8

This example describes production of linalool in S. marcescens HBQA7, which is superior to other S. marcescens hosts.

Strains S07, S08, S09, S10, S11, S12, S13, S14, S15, S16, S17, S18, S19, S20, and S21 were streaked and cultured on LB solid media. A single colony of each of the strains S07, S08, S09, S10, S11, S12, S13, S14, S15, S16, S17, S18, S19, S20, and S21 was picked, and inoculated into 3 mL of a 2YT culture medium (peptone (16.0 g·L⁻¹), NaCl (5.0 g·L⁻¹), and yeast extract (10.0 g·L⁻¹)). The strains were cultured overnight at 30° C., 200 rpm. Then, the strains were inoculated into 50 ml of a fresh 2YT culture medium to which glucose (60 g·L⁻¹) was added at an inoculum concentration of 2% (by volume). To collect linalool, dodecane (10% by volume) was added to the culture medium. The strains were cultured at 30° C., 120 rpm for 72 h. An OD value was measured at 600 nm using a spectrophotometer. The linalool in the dodecane was detected by gas chromatography. The OD values of the strains S07, S08, S09, S10, S11, S12, S13, S14, S15, S16, S17, S18, S19, S20, and S21 were 5.86, 5.63, 6.05, 6.41, 5.91, 5.82, 6.06, 5.66, 5.90, 6.31, 5.72, 5.72, 6.26, 5.50, and 6.05, respectively, and the yields of linalool of the strains were 4.63 g·L⁻¹, 3.99 g·L⁻¹, 3.71 g·L⁻¹, 4.15 g·L⁻¹, 4.45 g·L⁻¹, 4.02 g·L⁻¹, 4.17 g·L⁻¹, 3.38 g·L⁻¹, 3.18 g·L⁻¹, 4.78 g·L⁻¹, 3.53 g·L⁻¹, 4.53 g·L⁻¹, 3.31 g·L⁻¹, 4.97 g·L⁻¹, and 4.20 g·L⁻¹, respectively. Compared to the strains S07, S08, S09, S10, S11, S12, S13, S14, S15, S16, S17, S18, S19, S20, and S21, the strain S06 has a higher yield of linalool, indicating that S. marcescens HBQA7 is superior to other S. marcescens hosts.

### Example 9

This example describes production of isoprene in S. marcescens HBQA7, which is superior to other S. marcescens hosts.

Strains S22, S23, S24, S25, S26, S27, S28, S29, S30, S31, S32, S33, S34, S35, S36, and S37 were streaked and cultured on LB solid media. A single colony of each of the strains S22, S23, S24, S25, S26, S27, S28, S29, S30, S31, S32, S33, S34, S35, S36, and S37 was picked, and inoculated into 3 mL of a 2YT culture medium (peptone (16.0 g·L⁻¹), NaCl (5.0 g·L⁻¹), and yeast extract (10.0 g·L⁻¹)). The strains were cultured overnight at 30° C., 200 rpm. Then, the strains were inoculated into 50 ml of a fresh 2YT culture medium to which glucose (60 g. L⁻¹) was added at an inoculum concentration of 2% (by volume). To collect isoprene, dodecane (10% by volume) was added to the culture medium. The strains were cultured at 30° C., 120 rpm for 72 h. An OD value was measured at 600 nm using a spectrophotometer. The isoprene in the dodecane layer was detected by gas chromatography. The OD values of the strains S22, S23, S24, S25, S26, S27, S28, S29, S30, S31, S32, S33, S34, S35, S36, and S37 were 5.72, 5.73, 5.52, 6.38, 5.82, 6.48, 5.53, 5.55, 5.60, 5.51, 6.15, 6.07, 5.91, 5.78, 6.03, and 5.97, respectively, and the yields of isoprene of the strains were 12.95 g·L⁻¹, 1.36 g·L⁻¹, 1.86 g·L⁻¹, 2.41 g·L⁻¹, 1.66 g·L⁻¹, 1.76 g·L⁻¹, 2.28 g·L⁻¹, 2.43 g·L⁻¹, 1.72 g·L⁻¹, 1.67 g·L⁻¹, 1.71 g·L⁻¹, 1.77 g·L⁻¹, 2.44 g·L⁻¹, 2.78 g·L⁻¹, 1.73 g·L⁻¹, and 2.56 g·L⁻¹, respectively. Compared to the strains S23, S24, S25, S26, S27, S28, S29, S30, S31, S32, S33, S34, S35, S36, and S37, the strain S22 has a higher yield of isoprene, indicating that S. marcescens HBQA7 is superior to other S. marcescens hosts.

### Example 10

This example describes production of isopentenol in S. marcescens HBQA7, which is superior to other S. marcescens hosts.

Strains S38, S39, S40, S41, S42, S43, S44, S45, S46, S47, S48, S49, S50, S51, S52, and S53 were streaked and cultured on LB solid media. A single colony of each of the strains S38, S39, S40, S41, S42, S43, S44, S45, S46, S47, S48, S49, S50, S51, S52, and S53 was picked, and inoculated into 3 mL of a 2YT culture medium (peptone (16.0 g·L⁻¹), NaCl (5.0 g·L⁻¹), and yeast extract (10.0 g·L⁻¹)). The strains were cultured overnight at 30° C., 200 rpm. Then, the strains were inoculated into 50 ml of a fresh 2YT culture medium to which glucose (60 g·L⁻¹) was added at an inoculum concentration of 2% (by volume). To collect isopentenol, dodecane (10% by volume) was added to the culture medium. The strains were cultured at 30° C., 120 rpm for 72 h. An OD value was measured at 600 nm using a spectrophotometer. The isopentenol in the dodecane layer was detected by gas chromatography. The OD values of the strains S38, S39, S40, S41, S42, S43, S44, S45, S46, S47, S48, S49, S50, S51, S52, and S53 were 5.68, 5.69, 5.53, 5.88, 5.83, 6.44, 5.81, 6.16, 6.49, 5.89, 6.01, 6.41, 5.91, 6.39, 6.15, and 6.30, respectively, and the yields of isopentenol of the strains were 11.79 g·L⁻¹, 2.34 g·L⁻¹, 2.29 g·L⁻¹, 2.28 g·L⁻¹, 1.80 g·L⁻¹, 1.19 g·L⁻¹, 1.17 g·L⁻¹, 1.26 g·L⁻¹, 1.34 g·L⁻¹, 1.39 g·L⁻¹, 1.46 g·L⁻¹, 1.49 g·L⁻¹, 2.22 g·L⁻¹, 1.61 g·L⁻¹, 1.44 g·L⁻¹, and 1.32 g·L⁻¹, respectively. Compared to the strains S39, S40, S41, S42, S43, S44, S45, S46, S47, S48, S49, S50, S51, S52, and S53, the strain S38 has a higher yield of isopentenol, indicating that S. marcescens HBQA7 is superior to other S. marcescens hosts.

### Example 11

This example describes production of 1,8-cineole in S. marcescens HBQA7, which is superior to other S. marcescens hosts.

Strains S54, S55, S56, S57, S58, S59, S60, S61, S62, S63, S64, S65, S66, S67, S68, and S69 were streaked and cultured on LB solid media. A single colony of each of the strains S54, S55, S56, S57, S58, S59, S60, S61, S62, S63, S64, S65, S66, S67, S68, and S69 was picked, and inoculated into 3 mL of a 2YT culture medium (peptone (16.0 g·L⁻¹), NaCl (5.0 g·L⁻¹), and yeast extract (10.0 g·L⁻¹)). The strains were cultured overnight at 30° C., 200 rpm. Then, the strains were inoculated into 50 mL of a fresh 2YT culture medium to which glucose (60 g·L⁻¹) was added at an inoculum concentration of 2% (by volume). To collect 1,8-cineole, dodecane (10% by volume) was added to the culture medium. The strains were cultured at 30° C., 120 rpm for 72 h. An OD value was measured at 600 nm using a spectrophotometer. The 1,8-cineole in the dodecane layer was detected by gas chromatography. The OD values of the strains S54, S55, S56, S57, S58, S59, S60, S61, S62, S63, S64, S65, S66, S67, S68, and S69 were 5.98, 5.76, 5.73, 6.45, 6.21, 6.24, 5.79, 6.13, 6.15, 5.60, 5.51, 5.66, 5.91, 6.42, 6.08, and 5.80, respectively, and the yields of 1,8-cineole of the strains were 11.67 g·L⁻¹, 2.40 g·L⁻¹, 2.90 g·L⁻¹, 1.78 g·L⁻¹, 1.57 g·L⁻¹, 1.83 g·L⁻¹, 1.66 g·L⁻¹, 2.92 g·L⁻¹, 1.59 g·L⁻¹, 2.27 g·L⁻¹, 2.86 g·L⁻¹, 2.32 g·L⁻¹, 1.62 g·L⁻¹, 2.41 g·L⁻¹, 1.67 g·L⁻¹, and 2.21 g·L⁻¹, respectively. Compared to the strains S55, S56, S57, S58, S59, S60, S61, S62, S63, S64, S65, S66, S67, S68, and S69, the strain S54 has a higher yield of 1,8-cineole, indicating that S. marcescens HBQA7 is superior to other S. marcescens hosts.

### Example 12

This example describes production of α-pinene in S. marcescens HBQA7, which is superior to other S. marcescens hosts.

Strains S70, S71, S72, S73, S74, S75, S76, S77, S78, S79, S80, S81, S82, S83, S84, and S85 were streaked and cultured on LB solid media. A single colony of each of the strains S70, S71, S72, S73, S74, S75, S76, S77, S78, S79, S80, S81, S82, S83, S84, and S85 was picked, and inoculated into 3 mL of a 2YT culture medium (peptone (16.0 g·L⁻¹), NaCl (5.0 g·L⁻¹), and yeast extract (10.0 g·L⁻¹)). The strains were cultured overnight at 30° C., 200 rpm. Then, the strains were inoculated into 50 ml of a fresh 2YT culture medium to which glucose (60 g·L⁻¹) was added at an inoculum concentration of 2% (by volume). To collect α-pinene, dodecane (10% by volume) was added to the culture medium. The strains were cultured at 30° C., 120 rpm for 72 h. An OD value was measured at 600 nm using a spectrophotometer. The α-pinene in the dodecane layer was detected by gas chromatography. The OD values of the strains S70, S71, S72, S73, S74, S75, S76, S77, S78, S79, S80, S81, S82, S83, S84, and S85 were 6.27, 5.69, 5.51, 5.82, 5.96, 6.39, 6.37, 5.86, 5.71, 5.60, 6.43, 5.56, 5.55, 6.31, 6.21, and 5.62, respectively, and the yields of α-pinene of the strains were 10.70 g·L⁻¹, 2.82 g·L⁻¹, 1.75 g·L⁻¹, 2.72 g·L⁻¹, 1.54 g·L⁻¹, 1.92 g·L⁻¹, 1.69 g·L⁻¹, 1.84 g·L⁻¹, 2.65 g·L⁻¹, 2.60 g·L⁻¹, 2.63 g·L⁻¹, 2.85 g·L⁻¹, 2.38 g·L⁻¹, 2.89 g·L⁻¹, 2.01 g·L⁻¹, and 1.53 g·L⁻¹, respectively. Compared to the strains S71, S72, S73, S74, S75, S76, S77, S78, S79, S80, S81, S82, S83, S84, and S85, the strain S70 has a higher yield of α-pinene, indicating that S. marcescens HBQA7 is superior to other S. marcescens hosts.

### Example 13

This example describes production of pinene in S. marcescens HBQA7.

A strain S86 was streaked and cultured on an LB solid medium. A single colony of the strain S86 was picked, and inoculated into 3 mL of a 2YT culture medium (peptone (16.0 g·L″ 1), NaCl (5.0 g·L⁻¹), and yeast extract (10.0 g·L⁻¹)). The strain was cultured overnight at 30° C., 200 rpm. Then, the strains were inoculated into 50 ml of a fresh 2YT culture medium to which glucose (60 g·L⁻¹) was added at an inoculum concentration of 2% (by volume). To collect pinene, dodecane (10% by volume) was added to the culture medium. The strains were cultured at 30° C., 120 rpm for 72 h. An OD value was measured at 600 nm using a spectrophotometer. The pinene in the dodecane layer was detected by gas chromatography. The OD value of the strain S86 was 5.91, and the yield of pinene of the strain was 10.15 g·L⁻¹.

### Example 14

This example describes production of γ-terpinene in S. marcescens HBQA7.

A strain S87 was streaked and cultured on an LB solid medium. A single colony of the strain S87 was picked, and inoculated into 3 mL of a 2YT culture medium (peptone (16.0 g·L″ 1), NaCl (5.0 g·L⁻¹), and yeast extract (10.0 g·L⁻¹)). The strain was cultured overnight at 30° C., 200 rpm. Then, the strains were inoculated into 50 ml of a fresh 2YT culture medium to which glucose (60 g·L⁻¹) was added at an inoculum concentration of 2% (by volume). To collect γ-terpinene, dodecane (10% by volume) was added to the culture medium. The strains were cultured at 30° C., 120 rpm for 72 h. An OD value was measured at 600 nm using a spectrophotometer. The γ-terpinene in the dodecane layer was detected by gas chromatography. The OD value of the strain S87 was 5.98, and the yield of γ-terpinene of the strain was 11.61 g·L⁻¹.

### Example 15

This example describes production of geraniol in S. marcescens HBQA7.

A strain S88 was streaked and cultured on an LB solid medium. A single colony of the strain S88 was picked, and inoculated into 3 mL of a 2YT culture medium (peptone (16.0 g·L″ 1), NaCl (5.0 g·L⁻¹), and yeast extract (10.0 g·L⁻¹)). The strain was cultured overnight at 30° C., 200 rpm. Then, the strains were inoculated into 50 ml of a fresh 2YT culture medium to which glucose (60 g·L⁻¹) was added at an inoculum concentration of 2% (by volume). To collect geraniol, dodecane (10% by volume) was added to the culture medium. The strains were cultured at 30° C., 120 rpm for 72 h. An OD value was measured at 600 nm using a spectrophotometer. The geraniol in the dodecane layer was detected by gas chromatography. The OD value of the strain S88 was 6.04, and the yield of geraniol of the strain was 11.88 g·L⁻¹.

### Example 16

This example describes production of (+)-limonene in S. marcescens HBQA7.

A strain S89 was streaked and cultured on an LB solid medium. A single colony of the strain S89 was picked, and inoculated into 3 ml of a 2YT culture medium (peptone (16.0 g·L″ 1), NaCl (5.0 g·L⁻¹), and yeast extract (10.0 g·L⁻¹)). The strain was cultured overnight at 30° C., 200 rpm. Then, the strains were inoculated into 50 ml of a fresh 2YT culture medium to which glucose (60 g·L⁻¹) was added at an inoculum concentration of 2% (by volume). To collect (+)-limonene, dodecane (10% by volume) was added to the culture medium. The strains were cultured at 30° C., 120 rpm for 72 h. An OD value was measured at 600 nm using a spectrophotometer. The (+)-limonene in the dodecane layer was detected by gas chromatography. The OD value of the strain S89 was 5.71, and the yield of (+)-limonene of the strain was 10.89 g·L⁻¹.

### Example 17

This example describes production of (−)-limonene in S. marcescens HBQA7.

A strain S90 was streaked and cultured on an LB solid medium. A single colony of the strain S90 was picked, and inoculated into 3 mL of a 2YT culture medium (peptone (16.0 g·L″ 1), NaCl (5.0 g·L⁻¹), and yeast extract (10.0 g·L⁻¹)). The strain was cultured overnight at 30° C., 200 rpm. Then, the strains were inoculated into 50 mL of a fresh 2YT culture medium to which glucose (60 g·L⁻¹) was added at an inoculum concentration of 2% (by volume). To collect (−)-limonene, dodecane (10% by volume) was added to the culture medium. The strains were cultured at 30° C., 120 rpm for 72 h. An OD value was measured at 600 nm using a spectrophotometer. The (−)-limonene in the dodecane layer was detected by gas chromatography. The OD value of the strain S90 was 6.23, and the yield of (−)-limonene of the strain was 11.04 g·L⁻¹.

### Example 18

This example describes production of myrcene in S. marcescens HBQA7.

A strain S91 was streaked and cultured on an LB solid medium. A single colony of the strain S91 was picked, and inoculated into 3 mL of a 2YT culture medium (peptone (16.0 g·L 1), NaCl (5.0 g·L⁻¹), and yeast extract (10.0 g·L⁻¹)). The strain was cultured overnight at 30° C., 200 rpm. Then, the strains were inoculated into 50 ml of a fresh 2YT culture medium to which glucose (60 g·L⁻¹) was added at an inoculum concentration of 2% (by volume). To collect myrcene, dodecane (10% by volume) was added to the culture medium. The strains were cultured at 30° C., 120 rpm for 72 h. An OD value was measured at 600 nm using a spectrophotometer. The myrcene in the dodecane layer was detected by gas chromatography. The OD value of the strain S91 was 6.45, and the yield of myrcene of the strain was 11.36 g·L⁻¹.

### Example 19

This example describes production of β-ocimene in S. marcescens HBQA7.

A strain S92 was streaked and cultured on an LB solid medium. A single colony of the strain S92 was picked, and inoculated into 3 mL of a 2YT culture medium (peptone (16.0 g·L″ 1), NaCl (5.0 g·L⁻¹), and yeast extract (10.0 g·L⁻¹)). The strain was cultured overnight at 30° C., 200 rpm. Then, the strains were inoculated into 50 ml of a fresh 2YT culture medium to which glucose (60 g·L⁻¹) was added at an inoculum concentration of 2% (by volume). To collect B-ocimene, dodecane (10% by volume) was added to the culture medium. The strains were cultured at 30° C., 120 rpm for 72 h. An OD value was measured at 600 nm using a spectrophotometer. The β-ocimene in the dodecane layer was detected by gas chromatography.

The OD value of the strain S92 was 5.80, and the yield of β-ocimene of the strain was 11.86 g·L⁻¹.

### Example 20

This example describes production of sabinene in S. marcescens HBQA7.

A strain S93 was streaked and cultured on an LB solid medium. A single colony of the strain S93 was picked, and inoculated into 3 mL of a 2YT culture medium (peptone (16.0 g·L″ 1), NaCl (5.0 g·L⁻¹), and yeast extract (10.0 g·L⁻¹)). The strain was cultured overnight at 30° C., 200 rpm. Then, the strains were inoculated into 50 ml of a fresh 2YT culture medium to which glucose (60 g·L⁻¹) was added at an inoculum concentration of 2% (by volume). To collect sabinene, dodecane (10% by volume) was added to the culture medium. The strains were cultured at 30° C., 120 rpm for 72 h. An OD value was measured at 600 nm using a spectrophotometer. The sabinene in the dodecane layer was detected by gas chromatography. The OD value of the strain S93 was 5.69, and the yield of sabinene of the strain was 10.43 g·L⁻¹.

### Example 21

This example describes production of (−)-α-bisabolol in S. marcescens HBQA7.

Strains S94, S95, S96, S97, S98, S99, S100, S101, S102, S103, S104, S105, S106, S107, S108, and S109 were streaked and cultured on LB solid media. A single colony of each of the strains S94, S95, S96, S97, S98, S99, S100, S101, S102, S103, S104, S105, S106, S107, S108, and S109 was picked, and inoculated into 3 mL of a 2YT culture medium (peptone (16.0 g·L⁻¹), NaCl (5.0 g·L⁻¹), and yeast extract (10.0 g·L⁻¹)). The strains were cultured overnight at 30° C., 200 rpm. Then, the strains were inoculated into 50 ml of a fresh 2YT culture medium to which glucose (60 g·L⁻¹) was added at an inoculum concentration of 2% (by volume). To collect (−)-α-bisabolol, dodecane (10% by volume) was added to the culture medium. The strains were cultured at 30° C., 120 rpm for 72 h. An OD value was measured at 600 nm using a spectrophotometer. The (−)-α-bisabolol in the dodecane layer was detected by gas chromatography. The OD values of the strains S94, S95, S96, S97, S98, S99, S100, S101, S102, S103, S104, S105, S106, S107, S108, and S109 were 5.72, 5.64, 5.55, 6.38, 5.60, 6.02, 5.79, 5.88, 5.75, 5.86, 6.22, 6.42, 6.06, 5.83, 6.21, and 6.29, respectively, and the yields of (−)-α-bisabolol of the strains were 10.08 g·L⁻¹, 2.36 g·L⁻¹, 2.68 g·L⁻¹, 2.64 g·L⁻¹, 2.91 g·L⁻¹, 2.33 g·L⁻¹, 1.65 g·L⁻¹, 2.71 g·L⁻¹, 2.97 g·L⁻¹, 2.19 g·L⁻¹, 2.65 g·L⁻¹, 1.90 g·L⁻¹, 2.07 g·L⁻¹, 1.92 g·L⁻¹, 1.68 g·L⁻¹, and 2.75 g·L⁻¹, respectively. Compared to the strains S95, S96, S97, S98, S99, S100, S101, S102, S103, S104, S105, S106, S107, S108, and S109, the strain S94 has a higher yield of (−)-α-bisabolol, indicating that S. marcescens HBQA7 is superior to other S. marcescens hosts.

### Example 22

This example describes production of farnesol in S. marcescens HBQA7, which is superior to other S. marcescens hosts.

Strains S110, S111, S112, S113, S114, S115, S116, S117, S118, S119, S120, S121, S122, S123, S124, and S125 were streaked and cultured on LB solid media. A single colony of each of the strains S110, S111, S112, S113, S114, S115, S116, S117, S118, S119, S120, S121, S122, S123, S124, and S125 was picked, and inoculated into 3 mL of a 2YT culture medium (peptone (16.0 g·L⁻¹), NaCl (5.0 g·L⁻¹), and yeast extract (10.0 g·L⁻¹)). The strains were cultured overnight at 30° C., 200 rpm. Then, the strains were inoculated into 50 ml of a fresh 2YT culture medium to which glucose (60 g·L⁻¹) was added at an inoculum concentration of 2% (by volume). To collect farnesol, dodecane (10% by volume) was added to the culture medium. The strains were cultured at 30° C., 120 rpm for 72 h. An OD value was measured at 600 nm using a spectrophotometer. The farnesol in the dodecane layer was detected by gas chromatography. The OD values of the strains S110, S111, S112, S113, S114, S115, S116, S117, S118, S119, S120, S121, S122, S123, S124, and S125 were 6.07, 6.15, 5.69, 6.13, 5.55, 6.26, 5.95, 6.02, 6.33, 6.29, 6.01, 5.77, 6.16, 5.98, 5.50, and 5.64, respectively, and the yields of farnesol of the strains were 10.33 g·L⁻¹, 2.80 g·L⁻¹, 2.21 g·L⁻¹, 1.85 g·L⁻¹, 2.37 g·L⁻¹, 2.10 g·L⁻¹, 1.79 g·L⁻¹, 1.60 g·L⁻¹, 1.67 g·L⁻¹, 2.32 g·L⁻¹, 2.46 g·L⁻¹, 1.89 g·L⁻¹, 2.22 g·L⁻¹, 1.83 g·L⁻¹, 2.18 g·L⁻¹, and 2.58 g·L⁻¹, respectively. Compared to the strains S111, S112, S113, S114, S115, S116, S117, S118, S119, S120, S121, S122, S123, S124, and S125, the strain S110 has a higher yield of farnesol, indicating that S. marcescens HBQA7 is superior to other S. marcescens hosts.

### Example 23

This example describes production of longifolene in S. marcescens HBQA7.

Strains S126, S127, S128, S129, S130, S131, S132, S133, S134, S135, S136, S137, S138, S139, S140, and S141 were streaked and cultured on LB solid media. A single colony of each of the strains S126, S127, S128, S129, S130, S131, S132, S133, S134, S135, S136, S137, S138, S139, S140, and S141 was picked, and inoculated into 3 mL of a 2YT culture medium (peptone (16.0 g·L⁻¹), NaCl (5.0 g·L⁻¹), and yeast extract (10.0 g·L⁻¹)). The strains were cultured overnight at 30° C., 200 rpm. Then, the strains were inoculated into 50 ml of a fresh 2YT culture medium to which glucose (60 g·L⁻¹) was added at an inoculum concentration of 2% (by volume). To collect longifolene, dodecane (10% by volume) was added to the culture medium. The strains were cultured at 30° C., 120 rpm for 72 h. An OD value was measured at 600 nm using a spectrophotometer. The longifolene in the dodecane layer was detected by gas chromatography. The OD values of the strains S126, S127, S128, S129, S130, S131, S132, S133, S134, S135, S136, S137, S138, S139, S140, and S141 were 6.16, 6.21, 6.49, 6.44, 5.58, 6.32, 5.80, 6.27, 6.40, 6.36, 6.09, 5.54, 6.33, 6.17, 5.50, and 5.75, respectively, and the yields of longifolene of the strains were 10.50 g·L⁻¹, 1.68 g·L⁻¹, 2.20 g·L⁻¹, 1.59 g·L⁻¹, 2.66 g·L⁻¹, 2.04 g·L⁻¹, 2.65 g·L⁻¹, 2.46 g·L⁻¹, 1.92 g·L⁻¹, 2.59 g·L⁻¹, 2.33 g·L⁻¹, 2.95 g·L⁻¹, 1.71 g·L⁻¹, 1.83 g·L⁻¹, 1.56 g·L⁻¹, and 1.88 g·L⁻¹, respectively. Compared to the strains S127, S128, S129, S130, S131, S132, S133, S134, S135, S136, S137, S138, S139, S140, and S141, the strain S126 has a higher yield of longifolene, indicating that S. marcescens HBQA7 is superior to other S. marcescens hosts.

### Example 24

This example describes production of valencene in S. marcescens HBQA7.

A strain S142 was streaked and cultured on an LB solid medium. A single colony of the strain S142 was picked, and inoculated into 3 mL of a 2YT culture medium (peptone (16.0 g·L⁻¹), NaCl (5.0 g·L⁻¹), and yeast extract (10.0 g·L⁻¹)). The strain was cultured overnight at 30° C., 200 rpm. Then, the strains were inoculated into 50 ml of a fresh 2YT culture medium to which glucose (60 g·L⁻¹) was added at an inoculum concentration of 2% (by volume). To collect valencene, dodecane (10% by volume) was added to the culture medium. The strains were cultured at 30° C., 120 rpm for 72 h. An OD value was measured at 600 nm using a spectrophotometer. The valencene in the dodecane layer was detected by gas chromatography. The OD value of the strain S142 was 6.22, and the yield of valencene of the strain was 10.10 g·L⁻¹.

### Example 25

This example describes production of B-elemene in S. marcescens HBQA7.

A strain S143 was streaked and cultured on an LB solid medium. A single colony of the strain S143 was picked, and inoculated into 3 mL of a 2YT culture medium (peptone (16.0 g·L⁻¹), NaCl (5.0 g·L⁻¹), and yeast extract (10.0 g·L⁻¹)). The strain was cultured overnight at 30° C., 200 rpm. Then, the strains were inoculated into 50 ml of a fresh 2YT culture medium to which glucose (60 g·L⁻¹) was added at an inoculum concentration of 2% (by volume). To collect B-elemene, dodecane (10% by volume) was added to the culture medium. The strains were cultured at 30° C., 120 rpm for 72 h. An OD value was measured at 600 nm using a spectrophotometer. The B-elemene in the dodecane layer was detected by gas chromatography. The OD value of the strain S143 was 5.75, and the yield of B-elemene of the strain was 10.72 g·L⁻¹.

### Example 26

This example describes production of farnesene in S. marcescens HBQA7.

A strain S144 was streaked and cultured on an LB solid medium. A single colony of the strain S144 was picked, and inoculated into 3 mL of a 2YT culture medium (peptone (16.0 g·L⁻¹), NaCl (5.0 g·L⁻¹), and yeast extract (10.0 g·L⁻¹)). The strain was cultured overnight at 30° C., 200 rpm. Then, the strains were inoculated into 50 mL of a fresh 2YT culture medium to which glucose (60 g·L⁻¹) was added at an inoculum concentration of 2% (by volume). To collect farnesene, dodecane (10% by volume) was added to the culture medium. The strains were cultured at 30° C., 120 rpm for 72 h. An OD value was measured at 600 nm using a spectrophotometer. The farnesene in the dodecane layer was detected by gas chromatography. The OD value of the strain S144 was 5.83, and the yield of farnesene of the strain was 10.61 g·L″

### Example 27

This example describes production of patchoulol in S. marcescens HBQA7.

A strain S145 was streaked and cultured on an LB solid medium. A single colony of the strain S145 was picked, and inoculated into 3 mL of a 2YT culture medium (peptone (16.0 g·L⁻¹), NaCl (5.0 g·L⁻¹), and yeast extract (10.0 g·L⁻¹)). The strain was cultured overnight at 30° C., 200 rpm. Then, the strains were inoculated into 50 ml of a fresh 2YT culture medium to which glucose (60 g·L⁻¹) was added at an inoculum concentration of 2% (by volume). To collect patchoulol, dodecane (10% by volume) was added to the culture medium. The strains were cultured at 30° C., 120 rpm for 72 h. An OD value was measured at 600 nm using a spectrophotometer. The patchoulol in the dodecane layer was detected by gas chromatography. The OD value of the strain S145 was 5.56, and the yield of patchoulol was 10.38 g. L⁻¹.

### Example 28

This example describes production of pentalenene in S. marcescens HBQA7.

A strain S146 was streaked and cultured on an LB solid medium. A single colony of the strain S146 was picked, and inoculated into 3 mL of a 2YT culture medium (peptone (16.0 g·L⁻¹), NaCl (5.0 g·L⁻¹), and yeast extract (10.0 g·L⁻¹)). The strain was cultured overnight at 30° C., 200 rpm. Then, the strains were inoculated into 50 mL of a fresh 2YT culture medium to which glucose (60 g·L⁻¹) was added at an inoculum concentration of 2% (by volume). To collect pentalenene, dodecane (10% by volume) was added to the culture medium. The strains were cultured at 30° C., 120 rpm for 72 h. An OD value was measured at 600 nm using a spectrophotometer. The pentalenene in the dodecane layer was detected by gas chromatography. The OD value of the strain S146 was 5.92, and the yield of pentalenene of the strain was 10.26 g·L⁻¹.

### Example 29

This example describes production of α-santalene in S. marcescens HBQA7.

A strain S147 was streaked and cultured on an LB solid medium. A single colony of the strain S147 was picked, and inoculated into 3 mL of a 2YT culture medium (peptone (16.0 g·L⁻¹), NaCl (5.0 g·L⁻¹), and yeast extract (10.0 g·L⁻¹)). The strain was cultured overnight at 30° C., 200 rpm. Then, the strains were inoculated into 50 ml of a fresh 2YT culture medium to which glucose (60 g·L⁻¹) was added at an inoculum concentration of 2% (by volume). To collect α-santalene, dodecane (10% by volume) was added to the culture medium. The strains were cultured at 30° C., 120 rpm for 72 h. An OD value was measured at 600 nm using a spectrophotometer. The α-santalene in the dodecane layer was detected by gas chromatography. The OD value of the strain S147 was 5.67, and the yield of α-santalene of the strain was 11.40 g·L⁻¹.

### Example 30

The strain S06 was cultured in a 30 L fermenter. The strain S06 was streaked and cultured on an LB solid medium. A single colony of the strain S06 was picked, and inoculated into 3 mL of a 2YT culture medium (peptone (16.0 g·L⁻¹), NaCl (5.0 g·L⁻¹), and yeast extract (10.0 g·L⁻¹)). The strain was cultured at 30° C., 200 rpm for 12 h to obtain a primary seed liquid. The primary seed liquid was transferred into a 500 mL shake flask containing 100 ml of a 2YT culture medium at an inoculum concentration of 2% (by volume), and cultured under the same condition for 12 h to obtain a secondary seed liquid. Then the secondary seed liquid was transferred into a 2.5 L fermenter containing 1 L of a 2YT culture medium at an inoculum concentration of 5% (by volume), to obtain a tertiary seed liquid. Finally, the strain was cultured in a 30 L fermenter, with 20 L of an initial culture medium, at an initial glucose concentration of 60 g·L⁻¹, an initial pH of 7.0, a culture temperature of 30° C., an initial rotation speed of 200 rpm, and a ventilation rate of 1 vvm. At this time, the dissolved oxygen (DO) in the fermenter was calibrated to 100%. The seed liquid is inoculated into the fermenter at an inoculum concentration of 5% by volume (OD value of 23.54). To control the dissolved oxygen in the fermenter to not be lower than 30%, the dissolved oxygen needs to be coupled with the rotation speed throughout the fermentation process. When batch fermentation proceeded for about 8 h, the dissolved oxygen rebounded. At this time, a carbon source in the initial culture medium was basically exhausted, and feeding was carried out in a dissolved oxygen linked fed-batch manner. When the dissolved oxygen was greater than 80%, feeding was carried out. When the dissolved oxygen dropped to less than 60%, feeding was stopped. Throughout the fermentation process, ammonia water (25%) was fed for pH adjustment to maintain the pH of a fermentation broth in the fermenter at about 7.0. The fermentation time was 180 h, and dodecane (10%) was used as an extractant. The fermentation medium contains the following substances: Na₂HPO₄·12H₂O (17.1 g·L⁻¹), KH₂PO₄ (3.0 g·L⁻¹), NaCl (5.0 g·L⁻¹), NH₄Cl (1.0 g·L⁻¹), yeast extract (10.0 g·L⁻¹), citric acid (0.2 g·L⁻¹), MgSO₄ (1.0 mM), CaCl₂) (0.1 mM), thiamine hydrochloride (0.008 g·L⁻¹), D-(+)-biotin (0.008 g·L⁻¹), nicotinic acid (0.008 g·L⁻¹), pyridoxine (0.032 g·L⁻¹), and a trace metal solution (1 mL·L⁻¹). The trace metal solution contains the following substances: NaCl (10 g·L⁻¹), citric acid (40 g·L⁻¹), ZnSO₄·7H₂O (1.0 g·L⁻¹), MnSO₄·H₂O (30 g·L⁻¹), CuSO₄: 5H₂O (0.1 g·L⁻¹), H₃BO₃ (0.1 g·L⁻¹), Na₂MoO₄: 2H₂O (0.1 g·L⁻¹), FeSO₄·7H₂O (1.0 g·L⁻¹), and CoCl₂·6H₂O (1.0 g·L⁻¹). A feeding medium contains: glucose (600 g·L⁻¹), KH₂PO₄ (9 g·L⁻¹), MgSO₄ (2.5 g·L⁻¹), K₂SO₄ (3.5 g·L⁻¹), Na₂SO₄ (0.28 g·L⁻¹), thiamine hydrochloride (0.012 g·L⁻¹), D-(+)-biotin (0.012 g·L⁻¹), nicotinic acid (0.012 g·L⁻¹), pyridoxine (0.012 g·L⁻¹), p-aminobenzoic acid (0.012 g·L⁻¹), and a trace metal solution (10 mL·L⁻¹). The linalool in the dodecane layer was detected by gas chromatography. The ultimate yield of linalool of the strain S06 with an OD value of 84.93 was 40.72 g·L⁻¹.

The disclosure has been disclosed above with preferred embodiments, but the disclosure is not limited thereby. Anyone familiar with this technology can make various changes and modifications without departing from the spirit and scope of the disclosure, and therefore, the protection scope of the disclosure should be based on the definition in the claims.

What is claimed is:

1. An engineered strain of Serratia marcescens, wherein Serratia marcescens HBQA7 is used as a chassis cell to express acetyl-CoA acetyltransferase, HMG-COA synthase, HMG-COA reductase, mevalonate kinase, phosphomevalonate kinase, and mevalonate pyrophosphate decarboxylase; and
the accession number of the Serratia marcescens HBQA7 preserved at the China Center for Type Culture Collection is CCTCC NO: M 2023184.

2. The engineered strain of Serratia marcescens according to claim 1, further expressing any one of enzymes or a combination thereof:
(1) isopentenyl pyrophosphate isomerase, geranyl pyrophosphate synthase, and any one of the following enzymes:
(R)-linalool synthase, 1,8-cineole synthase, α-pinene synthase, pinene synthase, γ-terpinene synthase, geraniol synthase, (+)-limonene synthase, (−)-limonene synthase, myrcene synthase, β-ocimene synthase, or sabinene synthase;
(2) isopentenyl pyrophosphate isomerase, farnesyl pyrophosphate synthase, and any one of the following enzymes:
(−)-α-bisabolol synthase, phosphatase, longifolene synthase, valencene synthase, germacrene A synthase, farnesene synthase, patchoulol synthase, pentalenene synthase, or α-santalene synthase; and
(3) isoprene synthase or isopentenol synthase.

3. The engineered strain of Serratia marcescens according to claim 2, wherein the amino acid sequence of the acetyl-CoA acetyltransferase is set forth in SEQ ID NO:1, the amino acid sequence of the HMG-COA synthase is set forth in SEQ ID NO:2, the amino acid sequence of the HMG-COA reductase is set forth in SEQ ID NO:3, the amino acid sequence of the mevalonate kinase is set forth in SEQ ID NO:4, the amino acid sequence of the phosphomevalonate kinase is set forth in SEQ ID NO:5, the amino acid sequence of the mevalonate pyrophosphate decarboxylase is set forth in SEQ ID NO:6, the amino acid sequence of the isopentenyl pyrophosphate isomerase is set forth in SEQ ID NO:7, the amino acid sequence of the geranyl pyrophosphate synthase is set forth in SEQ ID NO:8, the amino acid sequence of the (R)-linalool synthase is set forth in SEQ ID NO:9, the amino acid sequence of the 1,8-cineole synthase is set forth in SEQ ID NO:10, the amino acid sequence of the α-pinene synthase is set forth in SEQ ID NO:11, the amino acid sequence of the pinene synthase is set forth in SEQ ID NO:12, the amino acid sequence of the γ-terpinene synthase is set forth in SEQ ID NO:13, the amino acid sequence of the geraniol synthase is set forth in SEQ ID NO:14, the amino acid sequence of the (+)-limonene synthase is set forth in SEQ ID NO:15, the amino acid sequence of the (−)-limonene synthase is set forth in SEQ ID NO:16, the amino acid sequence of the myrcene synthase is set forth in SEQ ID NO:17, the amino acid sequence of the β-ocimene synthase is set forth in SEQ ID NO:18, the amino acid sequence of the sabinene synthase is set forth in SEQ ID NO:19, the amino acid sequence of the (−)-α-bisabolol synthase is set forth in SEQ ID NO:20, the amino acid sequence of the farnesyl pyrophosphate synthase is set forth in SEQ ID NO:21, the amino acid sequence of the phosphatase is set forth in SEQ ID NO:22, the amino acid sequence of the longifolene synthase is set forth in SEQ ID NO:23, the amino acid sequence of the valencene synthase is set forth in SEQ ID NO:24, the amino acid sequence of the germacrene A synthase is set forth in SEQ ID NO:25, the amino acid sequence of the farnesene synthase is set forth in SEQ ID NO:26, the amino acid sequence of the patchoulol synthase is set forth in SEQ ID NO:27, the amino acid sequence of the pentalenene synthase is set forth in SEQ ID NO:28, the amino acid sequence of the α-santalene synthase is set forth in SEQ ID NO:29, the amino acid sequence of the isoprene synthase is set forth in SEQ ID NO:30, and the amino acid sequence of the isopentenol synthase is set forth in SEQ ID NO:31.

4. The engineered strain of Serratia marcescens according to claim 3, wherein an expression vector used comprises pBBR1MCS-2, pBbA5c-RFP, or pUCP18.

5. A method for producing terpenoids using the engineered strain of Serratia marcescens according to claim 1, comprising: seeding a fermentation medium with the engineered strain of Serratia marcescens for fermentation at 30° C., 200 rpm for at least 72 hours.

6. The method according to claim 5, wherein the fermentation medium comprises glucose, Na₂HPO₄: 12H₂O, KH₂PO₄, NaCl, NH₄Cl, yeast extract, citric acid, MgSO₄, CaCl₂), thiamine hydrochloride, D-(+)-biotin, nicotinic acid, pyridoxine, and trace metals; and
the trace metals comprise Na⁺, Zn²⁺, Mn²⁺, Cu²⁺, B³⁺, Mo⁶⁺, Fe²⁺, and Co²⁺.

7. The method according to claim 6, wherein during fermentation, feeding is carried out, and a culture medium used for feeding comprises glucose, KH₂PO₄, MgSO₄, K₂SO₄, Na₂SO₄, thiamine hydrochloride, D-(+)-biotin, nicotinic acid, pyridoxine, p-aminobenzoic acid, and trace metals.

8. The method according to claim 7, wherein the pH of a fermentation broth is adjusted to 6 to 8 with ammonia water.

9. The method according to claim 8, wherein dodecane is added to the culture medium.
